(19) United States
(12) Patent Application Publication
(10) Pub. No.: US 2026/0325477 A1
(43) Pub. Date: Oct. 22, 2026

(54) METHODS OF TREATMENT USING DRY POWDER COMPOSITIONS OF TREPROSTINIL PRODRUGS

(71) Applicant: Insmed Incorporated, Bridgewater, NJ (US)

(72) Inventors: Ariel TEPER, Bridgewater, NJ (US); Eugene SULLIVAN, Bridgewater, NJ (US); Carlos FERNANDEZ, Bridgewater, NJ (US); Gerald O’BRIEN, Bridgewater, NJ (US); Fraz ISMAT, Bridgewater, NJ (US); Natalya MAKULOVA, Bridgewater, NJ (US)

(21) Appl. No.: 19/694,755

(22) Filed: Jun. 1, 2026

Related U.S. Application Data
(63) Continuation of application No. PCT/US2025/010303, filed on Jan. 3, 2025.
(60) Provisional application No. 63/617,653, filed on Jan. 4, 2024.
(60) Provisional application No. 63/713,970, filed on Oct. 30, 2024.
(60) Provisional application No. 63/724,111, filed on Nov. 22, 2024.

(51) Int. Cl. A61K 31/216; A61K 9/00; A61P 9/12

(52) U.S. Cl. CPC A61K 31/216; A61K 9/0075; A61P 9/12

(57) ABSTRACT

The present disclosure provides methods for treating pulmonary hypertension (PH) in a patient in need thereof, comprising administering via a dry powder inhaler (DPI) to the patient, during an administration period, a compound of Formula (I),

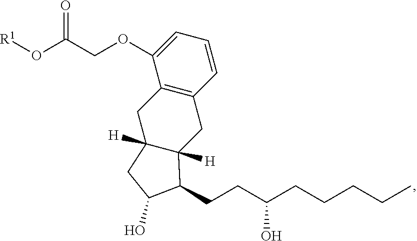

or an enantiomer, diastereomer, or a pharmaceutically acceptable salt thereof, wherein R¹ is tetradecyl, pentadecyl, hexadecyl, heptadecyl, or octadecyl. In embodiments, during the administration period, the compound of Formula (I), or an enantiomer, diastereomer, or a pharmaceutically acceptable salt thereof, is administered at a starting dose comprising about 80 μg to 160 μg once daily in a single dosing session, and the starting dose is titrated during the titration period to a maximum tolerated dose (MTD) of about 1280 μg or less. In embodiments, the administration period comprises a maintenance period, and the MTD is administered to the patient during the maintenance period. In embodiments, during the administration period, a dose of about 700-1300 μg of the compound of Formula (I), or enantiomer, diastereomer, or a pharmaceutically acceptable salt thereof, is administered once daily in a single dosing session to the patient.

### CROSS REFERENCE TO RELATED APPLICATIONS

This application claims priority to U.S. Provisional Application No. 63/617,653, filed on Jan. 4, 2024, U.S. Provisional Application No. 63/713,970, filed on Oct. 30, 2024, and U.S. Provisional Application No. 63/724,111, filed on Nov. 22, 2024, the contents of which are hereby incorporated by reference in their entireties for all purposes.

### BACKGROUND OF THE INVENTION

Pulmonary hypertension (PH) is characterized by an abnormally high blood pressure in the lung vasculature. It is a progressive, lethal disease that leads to heart failure and can occur in the pulmonary artery, pulmonary vein, or pulmonary capillaries. Symptomatic patients experience shortness of breath, dizziness, fainting, and other symptoms, all of which are made worse by exertion. There are multiple causes, and can be of unknown origin, idiopathic, and can lead to hypertension in other systems, for example, portopulmonary hypertension in which patients have both portal and pulmonary hypertension.

Pulmonary hypertension has been classified into five groups by the World Health Organization (WHO). Group 1 is called pulmonary arterial hypertension (PAH), and includes PAH that has no known cause (idiopathic), inherited PAH (i.e., familial PAH or FPAH), PAH that is caused by drugs or toxins, and PAH caused by conditions such as connective tissue diseases, HIV infection, liver disease, and congenital heart disease. Group 2 pulmonary hypertension is characterized as pulmonary hypertension associated with left heart disease. Group 3 pulmonary hypertension is characterized as PH associated with lung diseases, such as chronic obstructive pulmonary disease and interstitial lung diseases, as well as PH associated with sleep-related breathing disorders (e.g., sleep apnea). Group 4 PH is PH due to chronic thrombotic and/or embolic disease, e.g., PH caused by blood clots in the lungs or blood clotting disorders. Group 5 includes PH caused by other disorders or conditions, e.g., blood disorders (e.g., polycythemia vera, essential thrombocythemia), systemic disorders (e.g., sarcoidosis, vasculitis), and metabolic disorders (e.g., thyroid disease, glycogen storage disease).

Pulmonary arterial hypertension (PAH) afflicts approximately 200,000 people globally with approximately 30,000-40,000 of those patients in the United States. PAH patients experience constriction of pulmonary arteries which leads to high pulmonary arterial pressures, making it difficult for the heart to pump blood to the lungs. Patients suffer from shortness of breath and fatigue which often severely limits the ability to perform physical activity.

The New York Heart Association (NYHA) has categorized PAH patients into four functional classes to rate the severity of the disease. Class I PAIL patients as categorized by the NY-HA do not have a limitation of physical activity, as ordinary physical activity does not cause undue dyspnoea or fatigue, chest pain, or near syncope. Class II PAH patients as categorized by the NYHA have a slight limitation on physical activity. These patients are comfortable at rest, but ordinary physical activity causes undue dyspnoea or fatigue, chest pain or near syncope. Class III PAH patients as categorized by the NYHA have a marked limitation of physical activity. Although comfortable at rest, class III PAH patients experience undue dyspnoea or fatigue, chest pain or near syncope as a result of less than ordinary physical activity. Class IV PAH patients as categorized by the NYHA are unable to carry out any physical activity without symptoms. Class IV PAH patients might experience dyspnoea and/or fatigue at rest, and discomfort is increased by any physical activity. Signs of right heart failure are often manifested by class IV PAH patients.

Patients with PAR are treated with an endothelin receptor antagonist (ERA), phosphodiesterase type 5 (PDE-5) inhibitor, a guanylate cyclase stimulator, a prostanoid (e.g., prostacyclin), or a combination thereof. ERAs include ambrisentan (Letairis®), sitaxentan, bosentan (Tracleer®), and macitentan (Opsumit®). PDE-5 inhibitors indicated for the treatment of PAH include sildenafil (Revatio®) and tadalafil (Adcirca®). Prostanoids indicated for the treatment of PAH include iloprost, epoprostenol and treprostinil (Remodulin®, Tyvaso®). The one approved guanylate cyclase stimulator is riociguat (Adempas®). Additionally, patients are often treated with combinations of the aforementioned compounds.

The present invention addresses the need for novel treatment options for pulmonary hypertension (PH) (including pulmonary arterial hypertension (PAH) and PH associated with interstitial lung disease), portopulmonary hypertension (PPH), and pulmonary fibrosis by providing dry powder compositions of treprostinil prodrugs useful for pulmonary administration, and methods for administering the same to patients in need of treatment.

### SUMMARY OF THE INVENTION

In one aspect, the present disclosure relates to a method for treating pulmonary hypertension (PH) in a patient in need thereof, comprising administering via a dry powder inhaler (DPI) to the patient, once daily in a single dosing session during a titration period, a starting dose comprising about 80-160 μg of a compound of Formula (I),

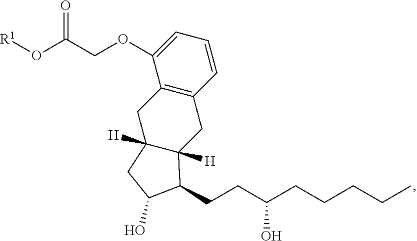

or an enantiomer, diastereomer, or a pharmaceutically acceptable salt thereof, wherein R¹ is tetradecyl, pentadecyl, hexadecyl, heptadecyl, or octadecyl, and then titrating the starting dose during the titration period to a maximum tolerated dose (MTD) of 1280 μg or less, administered once daily in a single dosing session. In a further embodiment, R¹ is hexadecyl. In one embodiment, the maximum tolerated dose is 1280 μg and R¹ is hexadecyl. In another embodiment, the maximum tolerated dose is 640 μg and R¹ is hexadecyl.

In another aspect, the present disclosure relates to a method for treating pulmonary hypertension (PH) in a patient in need thereof, comprising administering to the patient, via a dry powder inhaler (DPI), once daily in a single dosing session during an administration period, about 700 μg to about 1300 μg of a compound of Formula (I),

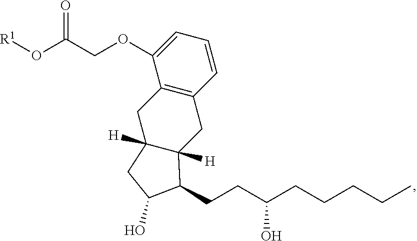

or an enantiomer, diastereomer, or a pharmaceutically acceptable salt thereof, wherein R¹ is tetradecyl, pentadecyl, hexadecyl, heptadecyl, or octadecyl. In a further embodiment, R¹ is hexadecyl. In embodiments, the patient is administered one or more dosages of about 720 μg, about 800 μg, about 880 μg, about 960 μg, about 1040 μg, about 1120 μg, about 1200 μg, or about 1280 μg of the compound of Formula (I), or an enantiomer, diastereomer, or a pharmaceutically acceptable salt thereof.

In yet another aspect, a method for treating pulmonary hypertension (PH) is provided. The method comprises administering to a patient in need of treatment, via a dry powder inhaler (DPI), once daily in a single dosing session during an administration period, a compound of Formula (I),

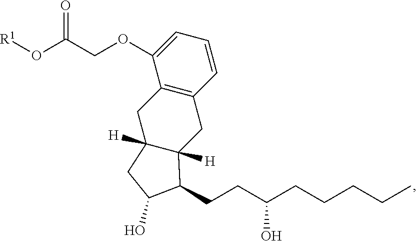

or an enantiomer, diastereomer, or a pharmaceutically acceptable salt thereof, wherein R¹ is tetradecyl, pentadecyl, hexadecyl, heptadecyl, or octadecyl,
wherein the administration period comprises (i) a titration period and (ii) a maintenance period, and wherein during the titration period, the starting dose comprising about 80-160 μg of the compound of Formula (I), or an enantiomer, diastereomer, or a pharmaceutically acceptable salt thereof is administered once daily in a single dosing session, and the starting dose is titrated to a maximum tolerated dose (MTD) of 1280 μg or less, and wherein during the maintenance period, the MTD is administered once daily in a single dosing session

In one embodiment of a method comprising a titration period, the titration period comprises an upwards dose titration. In another embodiment, the titration period comprises an upwards dose titration and a downwards dose titration. The upwards dose titration, in one embodiment, comprises increasing the dose of the compound of Formula (I), or enantiomer, diastereomer, or pharmaceutically acceptable salt thereof by an increment of about 80-160 μg. In a further embodiment, the upwards dose titration comprises increasing the dose of the compound of Formula (I), or enantiomer, diastereomer, or pharmaceutically acceptable salt thereof, by about a 80 μg increment. In another embodiment, the upwards dose titration comprises increasing the dose of the compound of Formula (I), or enantiomer, diastereomer, or pharmaceutically acceptable salt thereof, by about a 160 μg increment.

In embodiments, the starting dose is titrated to one or more doses selected from about 160 μg, about 240 μg, about 320 μg, about 400 μg, about 450 μg, about 480 μg, about 640 μg, about 720 μg, about 800 μg, about 880 μg, about 960 μg, about 1040 μg, about 1120 μg, about 1200 μg, or about 1280 μg. In another embodiment, the starting dose is titrated by increments of about 80 μg or 160 μg until achieving a dose of about 700-1300 μg. In one embodiment, the starting dose is 80 μg. In another embodiment, the starting dose is 160 μg. In one embodiment, a dose is titrated to a higher dose after the patient has shown to tolerate the dose for two or more days.

In some embodiments of a method comprising a titration period, the titration period is from about 20 to about 80 days.

In one embodiment of a method comprising a MTD, the MTD ranges from about 720 μg to about 1280 μg. In another embodiment, the MTD is about 720 μg, about 800 μg, about 880 μg, about 960 μg, about 1040 μg, about 1120 μg, about 1200 μg, or about 1280 μg.

During an upwards dose titration, in one embodiment, a patient remains on a dose of the compound of Formula (I), or enantiomer, diastereomer, or pharmaceutically acceptable salt thereof, for about one to seven days, e.g., from about two to about six days, from about two to about five days or from about two to about four days, prior to further incrementally increasing the dose. If a patient experiences an adverse event, for example a moderate or severe adverse event, then the patient is down-titrated to the previously administered dose, and the previously administered dose is then considered the maximally tolerated dose for the patient.

In one embodiment of a method provided herein, R¹ is tetradecyl. In a further embodiment, R¹ is linear tetradecyl.

In one embodiment of a method provided herein, R¹ is pentadecyl. In a further embodiment, R¹ is linear pentadecyl.

In one embodiment of a method provided herein, R¹ is heptadecyl. In a further embodiment, R¹ is linear heptadecyl.

In one embodiment of a method provided herein, R¹ is octadecyl. In a further embodiment, R¹ is linear octadecyl.

In a preferred embodiment of a method provided herein, R¹ is hexadecyl. In a further embodiment, R¹ is linear hexadecyl.

In one embodiment, the PH is group 1 PH, as characterized by the World Health Organization (WHO).

The pulmonary hypertension, in one embodiment, is pulmonary arterial hypertension (PAH). The PAH, in one embodiment, is class I PAH, as characterized by the New York Heart Association (NYHA). In another embodiment, the PAH is class II PAH, as characterized by NYHA. In another embodiment, the PAH is class III PAH, as characterized by NYHA. In another embodiment, the PAH is class IV PAH, as characterized by NYHA.

In another embodiment, the PH is group 2 PH, as characterized by the WHO. In another embodiment, the PH is group 3 PH, as characterized by the WHO. In a further embodiment, the group 3 PH is PH associated with interstitial lung disease (ILD). In another embodiment, the PH is group 4 PH, as characterized by the WHO. In another embodiment, the PH is group 5 PH, as characterized by the WHO.

In one embodiment of a method for treating PH in a patient in need thereof, the method comprises improving exercise capacity for the patient. In a further embodiment, the pulmonary hypertension is pulmonary arterial hypertension.

In one embodiment of a method for treating PH in a patient in need thereof, the method comprises improving exercise capacity for the patient. In a further embodiment, the pulmonary hypertension is pulmonary hypertension associated with interstitial lung disease.

In one embodiment of a method for treating PH in a patient in need thereof, wherein the treating comprises remodeling the pulmonary vasculature of the patient.

In some embodiments, the present disclosure provides a method of remodeling pulmonary vasculature in a patient with pulmonary hypertension associated with interstitial lung disease (PH-ILD), comprising administering via a dry powder inhaler (DPI) to the patient, once daily in a single dosing session during an administration period, an effective amount of a compound of Formula (I),

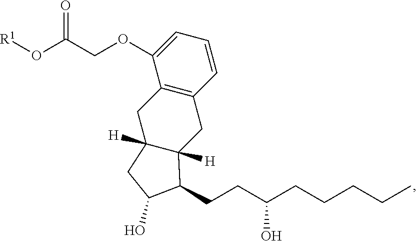

or an enantiomer, diastereomer, or a pharmaceutically acceptable salt thereof, wherein R¹ is tetradecyl, pentadecyl, hexadecyl, heptadecyl, or octadecyl.

In some embodiments, remodeling pulmonary vasculature comprises increasing the proportion of total blood volume in small pulmonary arteries during the administration period compared to the proportion of total blood volume in small pulmonary arteries prior to the administration period. In some embodiments, increasing the proportion of total blood volume in small pulmonary arteries comprises increasing by at least about 1%, at least about 5%, at least about 8%, at least about 10%, at least about 15%, or at least about 20% during the administration period compared to the proportion of total blood volume in small pulmonary arteries prior to the administration period. In some embodiments, increasing the proportion of total blood volume in small pulmonary arteries comprises increasing by about 5% to about 15% during the administration period compared to the proportion of total blood volume in small pulmonary arteries prior to the administration period. In some embodiments, increasing the proportion of total blood volume in small pulmonary arteries comprises increasing by about 8.4% during the administration period compared to the proportion of total blood volume in small pulmonary arteries prior to the administration period.

In some embodiments, remodeling pulmonary vasculature comprises increasing the proportion of total arterial volume in small pulmonary arteries during the administration period compared to the proportion of total arterial volume in small pulmonary arteries prior to the administration period. In some embodiments, increasing the proportion of total arterial volume in small pulmonary arteries comprises increasing by at least about 1%, at least about 5%, at least about 8%, at least about 10%, at least about 15%, or at least about 20% during the administration period compared to the proportion of total arterial volume in small pulmonary arteries prior to the administration period. In some embodiments, increasing the proportion of total arterial volume in small pulmonary arteries comprises increasing by about 5% to about 15% during the administration period compared to the proportion of total arterial volume in small pulmonary arteries prior to the administration period. In some embodiments, increasing the proportion of total arterial volume in small pulmonary arteries comprises increasing by about 8.0% during the administration period compared to the proportion of total arterial volume in small pulmonary arteries prior to the administration period.

In some embodiments, remodeling pulmonary vasculature comprises decreasing the proportion of total blood volume in large pulmonary arteries during the administration period compared to the proportion of total blood volume in large pulmonary arteries prior to the administration period. In some embodiments, decreasing the proportion of total blood volume in large pulmonary arteries comprises decreasing by at least about 1%, at least about 5%, at least about 10%, at least about 15%, or at least about 20% during the administration period compared to the proportion of total blood volume in large pulmonary arteries prior to the administration period. In some embodiments, decreasing the proportion of total blood volume in large pulmonary arteries comprises decreasing by about 5% to about 15% during the administration period compared to the proportion of total blood volume in large pulmonary arteries prior to the administration period. In some embodiments, decreasing the proportion of total blood volume in large pulmonary arteries comprises decreasing by about 10.8% during the administration period compared to the proportion of total blood volume in large pulmonary arteries prior to the administration period.

In some embodiments, remodeling pulmonary vasculature comprises decreasing the proportion of total arterial volume in large pulmonary arteries during the administration period compared to the proportion of total arterial volume in large pulmonary arteries prior to the administration period. In some embodiments, decreasing the proportion of total arterial volume in large pulmonary arteries comprises decreasing by at least about 1%, at least about 5%, at least about 10%, at least about 12%, at least about 15%, or at least about 20% during the administration period compared to the proportion of total arterial volume in large pulmonary arteries prior to the administration period. In some embodiments, decreasing the proportion of total arterial volume in large pulmonary arteries comprises decreasing by about 10% to about 20% during the administration period compared to the proportion of total arterial volume in large pulmonary arteries prior to the administration period. In some embodiments, decreasing the proportion of total arterial volume in large pulmonary arteries comprises decreasing by about 12% during the administration period compared to the proportion of total arterial volume in large pulmonary arteries prior to the administration period.

In some embodiments, remodeling pulmonary vasculature comprises increasing the ratio of total blood volume in small pulmonary arteries to total blood volume in large pulmonary arteries during the administration period compared to the ratio of total blood volume in small pulmonary arteries to total blood volume in large pulmonary arteries prior to the administration period. In some embodiments, increasing the ratio comprises increasing by at least about 10%, at least about 15%, at least about 20%, at least about 25%, at least about 30%, at least about 35%, at least about 40%, or at least about 50% during the administration period compared to the ratio prior to the administration period. In some embodiments, increasing the ratio comprises increasing by about 20% to about 40% during the administration period compared to the ratio prior to the administration period. In some embodiments, increasing the ratio comprises increasing by about 31% during the administration period compared to the ratio prior to the administration period.

In some embodiments, remodeling pulmonary vasculature comprises decreasing the specific image-based fibrosis (SIVFIB) score during the administration period compared to the SIVFIB score prior to the administration period. In some embodiments, decreasing the SIVFIB score comprises decreasing by at least about 1%, at least about 2%, at least about 3%, at least about 4%, at least about 5%, at least about 6%, at least about 7%, at least about 8%, at least about 9%, or at least about 10% during the administration period compared to the SIVFIB score prior to the administration period. In some embodiments, decreasing the SIVFIB score comprises decreasing by about 2% to about 10% during the administration period compared to the SIVFIB score prior to the administration period. In some embodiments, decreasing the SIVFIB score comprises decreasing by about 4.6% during the administration period compared to the SIVFIB score prior to the administration period.

In some embodiments, remodeling pulmonary vasculature comprises increasing the patient's lung function during the administration period compared to the patient's lung function prior to the administration period. In some embodiments, remodeling pulmonary vasculature further comprises decreasing the decline in lung function in the patient during the administration period.

In some embodiments, remodeling pulmonary vasculature further comprises improving exercise capacity of the patient during the administration period compared to the exercise capacity of the patient prior to the administration period.

In some embodiments, total blood volume is measured by functional respiratory imaging prior to the administration period and at week 16 of the administration period.

### BRIEF DESCRIPTION OF THE FIGURES

FIG. 1 provides non-limiting examples of dose titration schedules for administering 1280 μg of a compound of Formula (I) or (II).

FIG. 2 provides a non-limiting example of a dose titration schedule for administering 640 μg of a compound of Formula (I) or (II).

FIG. 3 provides an overall schema for an open-label extension study to assess safety, tolerability, and effectiveness of the long-term use of treprostinil palmityl inhalation powder (TPIP) in participants with pulmonary arterial hypertension (PAH).*3-week titration period for all participants; **4-week follow-up period for participants discontinuing study treatment; ***Participants may have their TPIP dose increased higher than 640 μg QD, up to a maximum dose of 1280 μg QD for events such as suspected disease progression or clinical worsening.

FIG. 4 provides an overall schema for a clinical study to evaluate the safety and tolerability of treprostinil palmityl inhalation powder (TPIP) in participants with pulmonary hypertension (PH) associated with interstitial lung disease (ILD).

### DETAILED DESCRIPTION OF THE INVENTION

Throughout the present disclosure, the term “about” may be used in conjunction with numerical values and/or ranges. The term “about” is understood to mean those values near to a recited value. For example, “about 40 [units]” may mean within ±25% of 40 (e.g., from 30 to 50), within ±20%, ±15%, ±10%, ±9%, ±8%, ±7%, ±6%, ±5%, +4%, ±3%, ±2%, ±1%, less than ±1%, or any other value or range of values therein or there below.

The term “pharmaceutically acceptable salt” refers to salts prepared from pharmaceutically acceptable non-toxic bases or acids including inorganic or organic bases and inorganic or organic acids. The nature of the salt is not critical, provided that it is pharmaceutically acceptable. Suitable pharmaceutically acceptable acid addition salts may be prepared from an inorganic acid or from an organic acid. Exemplary pharmaceutical salts are disclosed in Stahl, P. H., Wermuth, C. G., Eds. Handbook of Pharmaceutical Salts: Properties, Selection and Use; Verlag Helvetica Chimica Acta/Wiley-VCH: Zurich, 2002, the contents of which are hereby incorporated by reference in their entirety. Specific non-limiting examples of inorganic acids are hydrochloric, hydrobromic, hydroiodic, nitric, carbonic, sulfuric and phosphoric acid. Appropriate organic acids include, without limitation, aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric or galacturonic acid. Suitable pharmaceutically acceptable salts of free acid-containing compounds disclosed herein include, without limitation, metallic salts and organic salts. Exemplary metallic salts include, but are not limited to, appropriate alkali metal (group Ia) salts, alkaline earth metal (group IIa) salts, and other physiological acceptable metals. Such salts can be made from aluminum, calcium, lithium, magnesium, potassium, sodium and zinc. Exemplary organic salts can be made from primary amines, secondary amines, tertiary amines and quaternary ammonium salts, for example, tromethamine, diethylamine, tetra-N-methylammonium, N,N′-dibenzylethylenediamine, chloroprocaine, choline, diethanolamine, ethylenediamine, meglumine (N-methylglucamine) and procaine.

The term “stereoisomer” as used herein refers to two molecules having the same molecular formula and sequence of bonded atoms, but differ in three-dimensional orientations of their atoms in space. One preferred stereoisomer according to the present invention is a diastereomer. The stereoisomer, in one embodiment, is a diastereomer of a compound of Formula (I), or a pharmaceutically acceptable salt thereof. In a further embodiment, the stereoisomer is a diastereomer of a compound of Formula (I). In another embodiment, the stereoisomer is a diastereomer of a pharmaceutically acceptable salt of a compound of Formula (I). In yet another embodiment, the stereoisomer is a diastereomer of a compound of Formula (II). In even another embodiment, the stereoisomer is a diastereomer of a pharmaceutically acceptable salt of a compound of Formula (II).

Throughout the present specification, numerical ranges are provided for certain quantities. It is to be understood that these ranges comprise all subranges therein. Thus, the range “50-80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

Throughout the present specification, numerical ranges are described as encompassing “about 80% to about 125%” or “about 80-125%” of a range of values. It is to be understood that these comprise 80% of the lowest endpoint of the range up to 125% of the highest endpoint of the range, and all values therein.

The term “Cₘₐₓ” means the maximum (or peak) treprostinil serum concentration measured after a compound of Formula (I) or (II), an enantiomer or diastereomer thereof, or a pharmaceutically acceptable salt thereof is administered to the lungs of a subject via a dry powder composition described herein. In addition, Cₘₐₓ may be measured after a single administration of a compound of Formula (I) or (II), an enantiomer or diastereomer thereof, or a pharmaceutically acceptable salt thereof, described herein, or treprostinil Cₘₐₓ may be measured at steady state. Unless stated otherwise, Cₘₐₓ refers to the average treprostinil Cₘₐₓ measured after a single administration among a population of subjects (e.g., a population of PH patients).

The term “AUC” means the area under the plasma concentration time curve for treprostinil, measured from time 0 to a certain time post-administration to the lungs of a subject, calculated by a combination of linear and logarithmic trapezoidal methods (Linear up/log down method). In some embodiments, AUC may be measured from time 0 to 24 hours post-administration (“AUC₀₋₂₄”) or AUC may be measured form from time 0 to extrapolated to infinity (“AUC_{0-inf}”). In addition, treprostinil AUC may be measured after a single administration or at steady state values. Unless stated otherwise, AUC refers to the average AUC measured after a single administration among a population of subjects (e.g., a population of PH patients).

The term “adult” refers to a human subject, e.g., a human patient that is at least 18 years of age or older. In some embodiments, the adult is 18-100 years of age, e.g., 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, including all values and ranges in between.

The term “treating” includes: (1) preventing or delaying the appearance of clinical symptoms of the state, disorder or condition developing in the patient that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); and/or (3) relieving the condition (e.g., causing regression of the state, disorder or condition or at least one of its clinical or subclinical symptoms). In one embodiment, “treating” refers to inhibiting the state, disorder or condition (e.g., arresting, reducing or delaying the development of the disease, or a relapse thereof in case of maintenance treatment, of at least one clinical or subclinical symptom thereof). In another embodiment, “treating” refers to relieving the condition (for example, by causing regression of the state, disorder or condition or at least one of its clinical or subclinical symptoms). The benefit to a patient to be treated is either statistically significant as compared to the state or condition of the same patient before the treatment, or as compared to the state or condition of an untreated control patient, or the benefit is at least perceptible to the patient or to the physician.

“Effective amount” means an amount of a compound of Formula (I), or an enantiomer, diastereomer, or pharmaceutically acceptable salt thereof that is sufficient to result in the desired therapeutic response. In one embodiment, the “effective amount” is the amount of the compound of Formula (I) or an enantiomer, diastereomer, or pharmaceutically acceptable salt thereof (e.g., a compound of Formula (II)) that is administered in a single dosing session.

As used herein “titration period” refers to a period of time when a starting dose of a compound of Formula (I), or enantiomer, diastereomer, or pharmaceutically acceptable salt thereof, is administered to a patient, and the starting dose is increased (titrated) incrementally until the patient reaches their maximum tolerated dose (MTD). The incremental increase in one embodiment, is by about 80 μg to about 200 μg, for example by about 80 μg or by about 160 μg. In a preferred embodiment, the incremental increase is by about 80 μg. In a preferred embodiment, the incremental increase is by about 160 μg. In addition to a dose being increased during a titration period, a dose Y may also be decreased to a prior dose X, e.g., if the patient experiences one or more adverse events at the dose Y of the compound of Formula (I), or enantiomer, diastereomer, or pharmaceutically acceptable salt thereof. In the embodiments described herein, a dose of the compound of Formula (I), or enantiomer, diastereomer, or pharmaceutically acceptable salt thereof is not further uptitrated once the patient is shown to tolerate a dose of 1280 μg.

As used herein, a “maintenance period” or “maintenance dosing period” refers to a period of time where the patient is administered an effective amount of a compound of Formula (I), or enantiomer, diastereomer, or pharmaceutically acceptable salt thereof, e.g., the patient's maximum tolerated dose (MTD) once daily during a single dosing session. As provided herein, the compound is administered via a dry powder inhaler (DPI). The compound, in one embodiment, is present in one of the dry powder compositions described herein, and can be present, in one embodiment, in a single or multiple DPI capsules.

The term “Functional Respiratory Imaging” or “FRI” refers to a non-invasive measurement of the patient-specific respiratory system. A set of distinct biomarkers analyzes exposure, structure, and function of the lungs and airways in a respiratory disease (e.g., PH-ILD). The regional information obtained by FRI is often crucial to understanding the pathophysiology of the patient and to give guidance for optimal treatment. In some embodiments, the FRI process comprises three phrases including medical imaging, image processing, and flow simulation. The process starts with the acquisition of low dose, high-resolution computed tomography (HRCT) scans of the patient (medical imaging). Measurements are then performed on the segmented 3-dimensional geometries from the scans (image processing) and computational fluid dynamics is then used to quantify airflow and exposure to inhaled particles (flow simulation).

As used herein, “small pulmonary arteries” refers to pulmonary arteries smaller than 5 mm² in a cross-sectional area.

As used herein, “large pulmonary arteries” refers to pulmonary arteries larger than 10 mm² in a cross-sectional area.

The present disclosure provides methods for treating pulmonary hypertension, e.g., pulmonary arterial hypertension (PAH) and pulmonary hypertension associated with interstitial lung disease (PH-ILD). In embodiments described herein, the methods entail improving the exercise capacity of the patient undergoing treatment.

In one aspect of the present invention, a method for treating pulmonary hypertension (PH) is provided. The method comprises administering to the patient in need of treatment, once daily during a single dosing session during a titration period, a starting dose of 80-160 μg a compound of Formula (I):

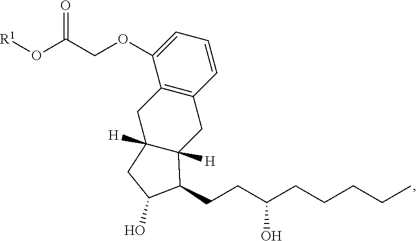

or an enantiomer, diastereomer, or a pharmaceutically acceptable salt thereof, wherein R¹ is tetradecyl, pentadecyl, hexadecyl, heptadecyl, or octadecyl, and wherein during the titration period, and then titrating the starting dose during the titration period to a maximum tolerated dose (MTD) of 1280 μg or less. In a further embodiment, the MTD is 700 μg, 720 μg, 800 μg, 880 μg, 960 μg, 1040 μg, 1120 μg, 1200 μg or 1280 μg. In a further embodiment, the MTD is 1280 μg. In embodiments of the method for treating PH, during the titration period, a dose X of the compound of Formula (I), or enantiomer, diastereomer or pharmaceutically acceptable salt thereof, is incrementally increased by about 80 μg to about 200 μg to a dose Y (wherein Y may be the MTD or an intermediate dose between the starting dose and the MTD), if the patient is shown to tolerate dose X. As used herein, “incremental increase”, “incrementally increased”, and the like, refer to a series of dose increases that are spread over time, wherein each dose in the series is administered for a period of time before administering a higher dose. In a further embodiment, a patient is administered a dose X for about one to about seven days prior to increasing the dose to dose Y. In a further embodiment, the patient is administered a dose X for about one to about five days prior to increasing the dose to dose Y. In even a further embodiment, the patient is administered a dose X for about one to about three days prior to increasing the dose to dose Y.

In some embodiments, during a titration period, the starting dose is 80 μg. In some embodiments, the starting dose is 160 μg. In some embodiments, the starting dose is incrementally increased (e.g., 80 μg or 160 μg) to the MTD. In some embodiments, the MTD is 720 μg-1280 μg.

In a preferred embodiment, the compound of Formula (I) or an enantiomer, diastereomer, or a pharmaceutically acceptable salt thereof, is present in a dry powder composition and is administered by a dry powder inhaler (DPI) during a titration period, once daily during a single dosing session.

In another aspect, the present disclosure relates to a method for treating pulmonary hypertension (PH) in a patient in need thereof, comprising administering via a dry powder inhaler, once daily during a single dosing session during an administration period, about 700 μg to about 1300 μg of a compound of Formula (I),

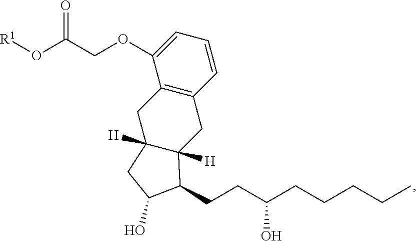

or an enantiomer, diastereomer, or a pharmaceutically acceptable salt thereof, wherein R¹ is tetradecyl, pentadecyl, hexadecyl, heptadecyl, or octadecyl. In some embodiments, a dose administered once daily during the single dosing session during the administration period is about 720 μg, about 800 μg, about 880 μg, about 960 μg, about 1040 μg, about 1120 μg, about 1200 μg, or about 1280 μg. In a further embodiment, substantially the same dose is administered to the patient during the administration period. In even a further embodiment, the dose is the patient's maximum tolerated dose (MTD).

In some embodiments, prior to administering about 700 μg to about 1300 μg of a compound of Formula (I), or an enantiomer, diastereomer, or a pharmaceutically acceptable salt thereof, the patient is administered a starting dose of about 80-160 μg of the compound of Formula (I), or an enantiomer, diastereomer, or a pharmaceutically acceptable salt thereof, once daily in a single dosing session. In some embodiments, after administering the starting dose for at least two days (e.g., 2-5 days) the starting dose is titrated incrementally to 1280 μg or less (e.g., if the patient's maximum tolerated dose is less than 1280 μg) during the titration period.

In yet another aspect, the method for treating pulmonary hypertension (PH) comprises administering to a patient in need of treatment, via a dry powder inhaler (DPI), once daily in a single dosing session during an administration period, a compound of Formula (I),

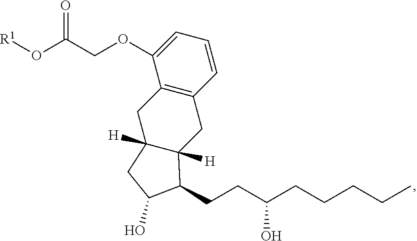

or an enantiomer, diastereomer, or a pharmaceutically acceptable salt thereof, wherein R¹ is tetradecyl, pentadecyl, hexadecyl, heptadecyl, or octadecyl, wherein the administration period comprises (i) a titration period and (ii) a maintenance period, and wherein during the titration period, the starting dose of 80-160 μg of the compound of Formula (I), or an enantiomer, diastereomer, or a pharmaceutically acceptable salt thereof is administered, and then the starting dose is titrated incrementally to a maximum tolerated dose (MTD) of 1280 μg or less, and wherein during the maintenance period, the MTD is administered to the patient.

In an embodiment of the compound of Formula (I), an enantiomer or diastereomer thereof, or a pharmaceutically acceptable salt thereof, R¹ is hexadecyl. In a further embodiment, R¹ is linear hexadecyl, i.e., the compound of Formula (I), an enantiomer or diastereomer thereof, or a pharmaceutically acceptable salt thereof, is a compound of Formula (II):

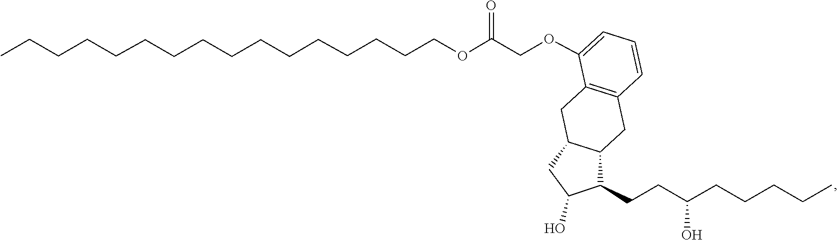 [SMILES: CCCCCCCCCCCCCCCCOC(=O)COc1cccc2c1C[C@H]1C[C@@H](O)[C@H](CC[C@@H](O)CCCCC)[C@H]1C2]

an enantiomer or diastereomer thereof, or a pharmaceutically acceptable salt thereof. In a further embodiment, the compound of Formula (I) is a compound of Formula (II). The compound of Formula (I) where R¹ is linear hexadecyl is also referred to herein as C16TR or its international nonproprietary name, treprostinil palmitil. In the present application, C16TR and treprostinil palmitil are used interchangeably. Similarly, a compound of Formula (II) is equivalent to a compound of Formula (I), wherein R¹ is linear hexadecyl. In one embodiment, the compound of Formula (I) is treprostinil palmitil having a CAS number of 1706528-83-7.

In a preferred embodiment, in the methods described herein, the compound of Formula (I) is treprostinil palmitil (i.e., R¹ is linear hexadecyl). In a further embodiment, the treprostinil palmitil is in a crystalline form. Crystalline forms of treprostinil palmitil are described in U.S. Pat. No. 10,781,160, which is incorporated herein by reference in its entirety.

In one embodiment, the treprostinil palmitil is a crystal Form I having an X-ray powder diffraction (XRPD) pattern exhibiting peaks at two, three, four, five or all of the following 2θ reflection angles: 3.3±10.2°, 6.6±0.2°, 14.2±0.2°, 18.9±0.2°, 21.3±0.2°, and 22.510.2°. In another embodiment, the XRPD pattern of treprostinil palmitil crystal Form I further comprises peaks at one, two, three, four, five, six, seven, eight or all of the following 2θ reflection angles: 13.8±0.2°, 15.3±0.2°, 16.9±0.20°, 17.8±0.2°, 19.8±0.2°, 20.6±0.2°, 20.9±0.20°, 24.4±0.2°, and 24.810.2°. In one embodiment, the treprostinil palmitil is a crystal Form I having an XRPD pattern exhibiting peaks at the 2θ reflection angles provided in Table 1.

TABLE 1
XRPD Data for Treprostinil Palmitil Crystal Form I.

| 2θ angle (°) | D value (Å) | Relative intensity (%) |
|---|---|---|
| 3.3 | 23.6 | 100 |
| 6.6 | 13.4 | 40.1 |
| 9.8 | 9.0 | 4.5 |
| 13.1 | 6.8 | 2.8 |
| 13.8 | 6.4 | 7.9 |
| 14.2 | 6.2 | 46.0 |
| 15.3 | 5.8 | 11.3 |
| 16.3 | 5.4 | 6.7 |
| 16.9 | 5.2 | 19.7 |
| 17.8 | 5.0 | 16.0 |
| 18.9 | 4.7 | 92.8 |
| 19.8 | 4.5 | 14.5 |
| 20.6 | 4.3 | 8.2 |
| 20.9 | 4.2 | 20.2 |
| 21.3 | 4.2 | 32.2 |
| 22.5 | 4.0 | 27.8 |
| 24.4 | 3.6 | 27.7 |
| 24.8 | 3.6 | 19.4 |
| 26.2 | 3.4 | 4.5 |
| 27.3 | 3.3 | 2.3 |
| 27.8 | 3.2 | 2.1 |
| 28.8 | 3.1 | 1.5 |
| 29.9 | 3.0 | 1.6 |
| 31.2 | 2.9 | 1.2 |
| 32.5 | 2.8 | 1.5 |
| 34.0 | 2.6 | 2.5 |
| 36.2 | 2.5 | 1.8 |
| 36.8 | 2.4 | 3.4 |
| 39.7 | 2.3 | 2.7 |

In one embodiment of a method described herein, the compound of Formula (I) is treprostinil palmitil and is treprostinil palmitil crystal Form I having a differential scanning calorimetry (DSC) thermogram pattern comprising an endothermic peak with a peak onset temperature of approximately 52.2±1 °C and a peak maximum of approximately 54.5±1 °C.

In one embodiment of a method described herein, treprostinil palmitil crystal Form II has an XRPD pattern exhibiting peaks at two, three, four, five or all of the following 2θ reflection angles: 3.4±0.2°, 6.1±0.2° 9.4±0.2°, 20.3±0.2°, 21.6±0.20°, and 23.4±0.2°. In another embodiment, the XRPD pattern of treprostinil palmitil crystal Form II further comprises peaks at one, two, three, four, five, six, seven, eight or all the following 2θ reflection angles: 7.0±0.2°, 9.0±0.2°, 12.2±0.2°, 12.7±0.2°, 17.5±0.2°, 18.0±0.2°, 18.5±0.2°, 19.1±0.2°, and 19.4±0.2°. In one embodiment, the treprostinil palmitil is a crystal Form II having an XRPD pattern exhibiting peaks at the 2θ reflection angles provided in Table 2.

TABLE 2
XRPD Data for Treprostinil Palmitil Crystal Form II.

| 2θ angle (°) | D value (Å) | Relative intensity (%) |
|---|---|---|
| 3.4 | 26.0 | 100.0 |
| 4.5 | 19.6 | 2.2 |
| 6.1 | 14.5 | 45.1 |
| 7.0 | 12.6 | 22.0 |
| 7.7 | 11.5 | 3.9 |
| 9.0 | 9.8 | 27.8 |
| 9.4 | 9.4 | 85.5 |
| 10.0 | 8.8 | 3.6 |
| 10.6 | 8.3 | 13.9 |
| 17.2 | 7.7 | 22.6 |
| 12.7 | 7.0 | 29.9 |
| 13.9 | 6.4 | 3.8 |
| 14.2 | 6.2 | 3.9 |
| 14.9 | 5.9 | 5.2 |
| 15.5 | 5.7 | 17.2 |
| 16.1 | 5.5 | 18.1 |
| 16.7 | 5.3 | 6.2 |
| 17.5 | 5.1 | 25.4 |
| 18.0 | 4.9 | 30.8 |
| 18.5 | 4.8 | 79.3 |
| 19.1 | 4.6 | 33.8 |
| 19.4 | 4.6 | 30.3 |
| 20.3 | 4.4 | 39.9 |
| 20.9 | 4.2 | 19.8 |
| 71.6 | 4.1 | 40.6 |
| 22.6 | 3.9 | 9.4 |
| 23.4 | 3.8 | 81.9 |
| 25.4 | 3.5 | 9.3 |
| 27.7 | 3.2 | 3.6 |
| 28.8 | 3.1 | 3.1 |
| 30.9 | 2.9 | 2.3 |
| 37.3 | 2.4 | 2.1 |
| 40.0 | 2.3 | 2.8 |
| 43.3 | 2.1 | 2.1 |

In one embodiment of a method for treating pulmonary hypertension provided herein, the compound of Formula (I) is treprostinil palmityl crystal Form II having a DSC thermogram pattern comprising an endothermic peak with a peak onset temperature of approximately 54.6±1 °C and a peak maximum of approximately 56.9±1 °C.

### Administration Period

In the methods provided herein, a patient is administered a prodrug of treprostinil, e.g., a compound of Formula (I), or an enantiomer, diastereomer, or a pharmaceutically acceptable salt thereof (e.g., treprostinil palmitil), for an administration period. In one embodiment, the administration is with food. The administration period, in one embodiment, comprises a titration period. In embodiments, during the titration period, the patient is administered a starting dose of a compound of Formula (I), or an enantiomer, diastereomer, or a pharmaceutically acceptable salt thereof (e.g., treprostinil palmitil), and the starting dose is titrated (e.g., incrementally increased) to an effective amount, e.g., a maximum tolerated dose (MTD). In one embodiment, the administration period comprises a maintenance period. The maintenance period is subsequent to the titration period and in some embodiments occurs once a MTD is established for a patient. In embodiments, during the maintenance period, the patient is administered the MTD once daily during a single dosing session. As discussed throughout, the maximum dose administered to patients with PH according to the methods disclosed herein is 1280 μg. Therefore, if a patient tolerates a dose of 1280 μg, then 1280 μg is considered the MTD. However, an individual MTD may be less than 1280 μg. Accordingly, in embodiments, a patient's MTD is selected from the group consisting of 720 μg, 800 μg, 880 μg, 960 μg, 1040 μg, 1120 μg, 1200 μg and 1280 μg.

In embodiments of a method for treating PH described herein, a patient is administered a compound of Formula (I), or an enantiomer, diastereomer, or a pharmaceutically acceptable salt thereof, once daily in a single dosing session during a titration period, e.g., via a dry powder inhaler. In one embodiment, the titration period comprises an upwards dose titration (also referred to as a uptitration). In embodiments, the upwards titration is initiated from a starting dose and the starting dose is incrementally increased until reaching the maximum tolerated dose. In another embodiment, the titration period comprises an upwards titration and a downwards titration (also referred to as a downtitration). Downtitration may occur if the patient does not tolerate the higher dose.

In embodiments of the method for treating PH, during the titration period, a dose X of the compound of Formula (I), or enantiomer, diastereomer or pharmaceutically acceptable salt thereof, is increased by about 80 μg to about 200 μg to a dose Y (e.g., by about 80 μg to about 160 μg or by about 80 μg), if the patient is shown to tolerate dose X. In this example, dose X may be the starting dose or any dose between the starting dose and the maximum tolerated dose, and dose Y may be the next sequential dose (e.g., a dose that is 80-160 μg higher than dose X), or dose Y may be the patient's maximum tolerated dose. In a further embodiment, the patient is administered the dose X for about one to about seven days prior to increasing the dose to dose Y. In a further embodiment, the patient is administered a dose X for about one to about five days prior to increasing the dose to dose Y. In even a further embodiment, the patient is administered a dose X for about one to about three days prior to increasing the dose to dose Y. In embodiments when dose Y is less than the maximum tolerated dose, the maximum tolerated dose may be achieved by increasing dose X by 80-160 μg to achieve dose Y and administering dose Y for a period of time (e.g., 2-7 days), then increasing dose Y by 80-160 μg, and repeating until reaching the maximum tolerated dose.

The upwards titration, in one embodiment, comprises increasing the dose of the compound of Formula (I), or enantiomer, diastereomer, or pharmaceutically acceptable salt thereof by increments of about 80-160 μg. In a further embodiment, the upwards dose titration, comprises increasing the dose of the compound of Formula (I), or enantiomer, diastereomer, or pharmaceutically acceptable salt thereof, by about 80 μg. In another embodiment, the upwards dose titration, comprises increasing the dose of the compound of Formula (I), or enantiomer, diastereomer, or pharmaceutically acceptable salt thereof, by about 160 μg. During an upwards dose titration, in one embodiment, a patient remains on a dose of the compound of Formula (I), or enantiomer, diastereomer, or pharmaceutically acceptable salt thereof, for about one to seven days, for example, one to six days prior to further incrementally increasing the dose. If a patient experiences an adverse event, then the patient is down-titrated to the prior dose, which can then be considered the maximally tolerated dose. In a preferred embodiment, the compound of Formula (I) is treprostinil palmitil.

During a titration period, a patient's dose can be gradually (in increments) uptitrated to the maximum tolerated dose for that patient. In some embodiment, the maximum tolerated dose is about 1280 μg. In another embodiment, a patient's maximum tolerated dose is selected from the group consisting of 720 μg, 800 μg, 880 μg, 960 μg, 1040 μg, 1120 μg, 1200 μg and 1280 μg. In some embodiments, the maximum tolerated dose is 1280 μg. In some embodiments, the maximum tolerated dose is 1200 μg. In some embodiments, the maximum tolerated dose is 1120 μg. In some embodiments, the maximum tolerated dose is 1040 μg. In some embodiments, the maximum tolerated dose is 960 μg. In some embodiments, the maximum tolerated dose is 880 μg. In some embodiments, the maximum tolerated dose is 800 μg. In some embodiments, the maximum tolerated dose is 720 μg. If a patient is uptitrated to a particular dose and then experiences one or more adverse events, for example, one or more moderate or severe adverse events, the dose of the compound of Formula (I), or enantiomer, diastereomer or pharmaceutically acceptable salt thereof, is downward titrated to the prior tolerated dose.

In embodiments, the upwards titration disclosed herein improves tolerability to the compound of Formula (I), or an enantiomer, diastereomer, or pharmaceutically acceptable salt thereof. In embodiments, the methods disclosed herein may be used with a patient who has not previously been administered a compound of Formula (I), or an enantiomer, diastereomer, or pharmaceutically acceptable salt thereof, is first administered a compound of Formula (I). In alternative embodiments, the methods disclosed herein may be used with a patient who resumes treatment with a compound of Formula (I), or an enantiomer, diastereomer, or pharmaceutically acceptable salt thereof, after previously discontinuing treatment for multiple days.

As an example of a titration period in one of the methods provided herein, a patient, in one embodiment, is initially administered a starting dose of 80 μg of a compound of Formula (I), or an enantiomer, diastereomer, or a pharmaceutically acceptable salt thereof, once daily during a single dosing session. In a further embodiment, the compound of Formula (I) is treprostinil palmitil and is present in a dry powder composition. The 80 μg of the compound of Formula (I), in one embodiment, is present in a dry powder composition, for example, in a single DPI capsule. After administering 80 μg the dose for several days (e.g., from 2-7 days) to the patient, the dose is gradually (in increments) uptitrated until reaching the patient's maximum tolerated dose. Uptitration, in one embodiment, comprises incrementally increasing the patient's dose of the compound of Formula (I) by about 80 or 160 μg after a patient is shown to tolerate a prior dose for about 2-7 days.

As another example of a titration period in one of the methods provided herein, a patient, in one embodiment, is initially administered a starting dose of 160 μg of a compound of Formula (I), or an enantiomer, diastereomer, or a pharmaceutically acceptable salt thereof, once daily during a single dosing session. In a further embodiment, the compound of Formula (I) is treprostinil palmitil and is present in a dry powder composition. The 160 μg of the compound of Formula (I), in one embodiment, is present in a dry powder composition, for example, in a single DPI capsule or multiple DPI capsules. After administering 160 μg the dose for several days (e.g., from 2-7 days) to the patient, the dose is gradually (in increments) uptitrated until reaching the patient's maximum tolerated dose. Uptitration, in one embodiment, comprises incrementally increasing the patient's dose of the compound of Formula (I) by about 80 or 160 μg after a patient is shown to tolerate a prior dose for about 2-7 days.

In embodiments, the maximum tolerated dose is the highest dose of the compound of Formula (I), or an enantiomer, diastereomer, or a pharmaceutically acceptable salt thereof administered to a patient for a period of time (e.g., at least 7-14 days) that does not cause one or more adverse events unrelated to the underlying condition. During a titration period, a patient's maximum tolerated dose does not exceed 1280 μg. In other words, the titration period ends when a patient is shown to tolerate a dose of 1280 μg. Thus, in some embodiments, the maximum tolerated dose is 1280 μg. In some embodiments, the maximum tolerated dose is 1200 μg. In some embodiments, the maximum tolerated dose is 1120 μg. In some embodiments, the maximum tolerated dose is 1040 μg. In some embodiments, the maximum tolerated dose is 960 μg. In some embodiments, the maximum tolerated dose is 880 μg. In some embodiments, the maximum tolerated dose is 800 μg. In some embodiments, the maximum tolerated dose is 720 μg.

In a further embodiment, the patient's maximum tolerated dose is administered to the patient as a maintenance dose to treat the pulmonary hypertension in the patient. For example, in an embodiment, the 1280 μg dose is administered to the patient as a maintenance dose to treat the pulmonary hypertension in the patient. In an embodiment, 1200 μg, 1120 μg, 1040 μg, 960 μg, 880 μg, 800 μg, or 720 μg is administered to the patient as a maintenance dose to treat the pulmonary hypertension in the patient. The maintenance dose is administered to the patient once daily during a single dosing session via a dry powder inhaler (DPI). The 1280 μg dose, in one embodiment, is present in multiple capsules for use with a capsule-based DPI.

During the titration period, patients stay on each dose of the compound of Formula (I) for a minimum number of cumulative days prior to starting the next higher dose (as a non-limiting example, 2 days at 80 μg, 160 μg, or 240 μg, 3 days at 320 μg or 4 days at 400 μg, 480 μg, 560 μg, 640 μg, 720 μg, 800 μg, 880 μg, 960 μg, 1040 μg, 1120 μg, 1200 μg or 1280 μg). After the titration period, the patient continues to administer the maximum tolerated dose (e.g., about 1280 μg), for a period of time during a maintenance treatment period.

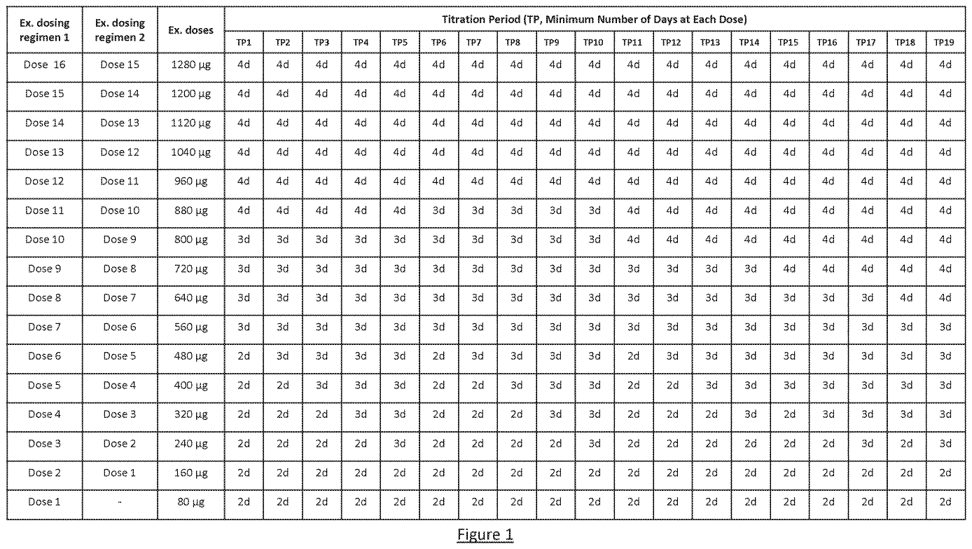

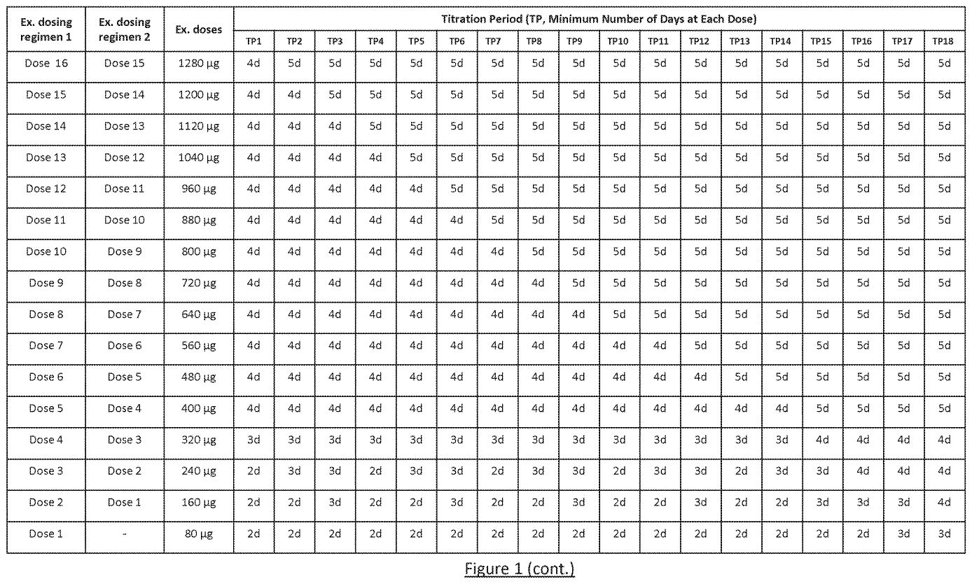

In embodiments, during the titration period, the minimum number of cumulative days during which patient stays on the same dose of a compound of Formula (I) ranges from 2-14 days, 2-7 days or 2-10 days. In embodiments, during a titration period, the minimum number of cumulative days during which patient stays on the same dose is 2 days, 3 days, 4 days, 5 days, 6 days, or 7 days, or any combination thereof, prior to titrating to the next higher dose. See, e.g., FIG. 1 for non-limiting embodiments of a dose titration period/dose titration scheme.

In some embodiments, the total length of the titration period is from about 20 days to about 90 days, from about 20 days to about 80 days, from about 20 days to about 60 days, from about 20 days to about 50 days, from about 25 days to about 75 days, or from about 25 days to about 60 days. In some embodiments, the total length of the titration period is about 2 weeks to about 6 weeks, about 2 weeks to about 5 weeks, about 2 weeks to about 3 weeks, about 3 weeks to about 6 weeks, about 3 weeks to about 5 weeks, or about 3 weeks to about 4 weeks. In some embodiments, the total length of the titration period is 21 days (i.e., three weeks). In some embodiments, the total length of the titration period is at least about 25 days, at least about 29 days, at least about 30 days, at least about 31 days, at least about 32 days, at least about 33 days, at least about 35 days, at least about 37 days, at least about 39 days, at least about 41 days, at least about 43 days, at least about 45 days, at least about 47 days, at least about 49 days, at least about 51 days, at least about 53 days, at least about 55 days, at least about 57 days, at least about 58 days, at least about 59 days, or at least about 60 days.

In some embodiments, during the titration period, the dose is uptitrated in increments of from about 10 μg to about 200 μg, e.g., about 10 μg, about 15 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, or about 200 μg until the patient's maximum tolerated dose is achieved. As described above, a titration period in one embodiment, is terminated once a patient is shown to tolerate a dose of 1280 μg. As such, a 1280 μg dose is considered a maximum tolerated dose for the methods described herein. In another embodiment, during the titration period, the dose is uptitrated in increments of about 80 μg or about 160 μg until the patient's maximum tolerated dose is achieved or a dose of 1280 μg is achieved. In yet another embodiment, during the titration period, the dose is uptitrated in increments of 80 μg until the patient's maximum tolerated dose is achieved or a dose of 1280 μg is achieved. In some embodiments, during the titration period, the patient receives one or more of a 720 μg dose, an 800 μg dose, an 880 μg dose, a 960 μg dose, a 1040 μg dose, a 1120 μg dose, a 1200 μg dose, and a 1280 μg dose. In some embodiments, the maximum tolerated dose of a patient undergoing a PH treatment method is 720 μg, 800 μg, 880 μg, 960 μg, 1040 μg, 1120 μg, 1200 μg, or 1280 μg.

In embodiments, the patient is shown to tolerate a dose before titrating to a higher dose. In some embodiments, a dose X is titrated to a higher dose Y after the patient has shown to tolerate the dose X for 2-14 days, 2-7 days, or 2-4 days. In some embodiments, a dose X is titrated to a higher dose Y after the patient has shown to tolerate the dose for 2 days, 3 days, 4 days, 5 days, 6 days, or 7 days. In one embodiment, a dose is titrated to a higher dose after the patient has shown to tolerate the dose for 2 days, 3 days, 4 days, or 5 days. In another embodiment, a dose X is titrated to a higher dose Y after the patient has shown to tolerate the dose for 2 days, 3 days, or 4 days. In one embodiment, a dose X is titrated to a higher dose Y after the patient has shown to tolerate the dose for 2 days. In one embodiment, a dose X is titrated to a higher dose Y after the patient has shown to tolerate the dose for 3 days. In one embodiment, a dose X is titrated to a higher dose Y after the patient has shown to tolerate the dose for 4 days. In one embodiment, a dose X is titrated to a higher dose Y after the patient has shown to tolerate the dose for 5 days. In one embodiment, a dose X is titrated to a higher dose Y after the patient has shown to tolerate the dose for 6 days. In one embodiment, a dose X is titrated to a higher dose Y after the patient has shown to tolerate the dose for 7 days. FIG. 1 provides an embodiment of dose titration for a patient in need of treatment. In one embodiment, if a dose Y is not tolerated, e.g., because the patient experiences one or more adverse events, the dose Y is downtitrated to the previous dose level X.

In some embodiments, a patient receiving a previously effective dose of a compound of Formula (I) may over time experience disease progression, clinical worsening of PH, or reduced efficacy of the dose that was previously effective. In embodiments, clinical worsening in a patient may be indicated by one or more of hospitalization for right heart failure, heart-lung or lung transplant, or atrial septostomy. In embodiments, clinical worsening may be indicated by a combined occurrence of two or more events including a 20% decrease in 6MWD, worsening WHO/NYHA functional capacity class, and/or appearance of or worsening of signs/symptoms of right heart failure. In embodiments, these patients may have their current dose titrated to higher dose, e.g., up to a maximum tolerated dose or if a dose of 1280 μg is achieved, to 1280 μg daily.

In embodiments, the previously effective dose ranges from 160-640 μg, 240-640 μg, 320-640 μg, 400-640 μg, 480-640 μg, or 560-640-640 μg of a compound of Formula (I) (e.g., treprostinil palmitil). In embodiments, the previously effective dose is 640 μg of a compound of Formula (I) (e.g., treprostinil palmitil), which was the highest dose administered in a clinical trial, and it was surprisingly observed to be well tolerated. To treat more severe cases of PH, e.g., PH-ILD, PAH or PAH that progresses after administering 640 μg, the dose of a compound of Formula (I) (e.g., treprostinil palmitil), the dose can be uptitrated to a higher dose, such as a maximum tolerated dose (MTD), e.g., an MTD of 1280 μg, whichever is less In some embodiments, the higher dose is 720 μg, 800 μg, 880 μg, 960 μg, 1040 μg, 1120 μg, 1200 μg, or 1280 μg. Is some embodiments, the dose is increased in 160 μg increments. Is some embodiments, the dose is increased in 80 μg increments.

If a dose is not tolerated, the dose may be decreased to the previous dose level. In some embodiments, a dose is considered not tolerated when a patient experiences one or more moderate adverse events (AEs). In some embodiments, a dose is considered not tolerated when a patient experiences one or more severe AEs. In some embodiments, a dose is considered not tolerated when a patient experiences one or more moderate to severe AEs.

An adverse event or AE refers to any untoward medical occurrence in a patient undergoing treatment for PH, wherein the AE is temporally associated with the administration of a compound of Formula (I) or Formula (II). An AE can therefore be any unfavorable and unintended sign, symptom, or disease (new or exacerbated) temporally associated with the use of a compound of Formula (I) or Formula (II). In some embodiments, the AE is a treatment-emergent adverse event (TEAE) defined as any AE that occurs after administration of a compound of Formula (I) or Formula (II).

AEs can be classified as mild, moderate, or severe as defined as follows:
Mild: An event that is easily tolerated by the participant, causing minimal discomfort and not interfering with everyday activities.
Moderate: An event that causes sufficient discomfort and interferes with normal everyday activities.
Severe: An event that prevents normal everyday activities.

In some embodiments, the AE is selected from cough, headache, diarrhea, nausea, rash (flushing), throat irritation/pharyngolaryngeal pain, pain in jaw, pain in extremity, hypokalemia, abdominal discomfort, tipper abdominal pain, dizziness, dyspnea, dyspepsia, vomiting, myalgia, eosinophilia, arthralgia, or syncope. In some embodiment, the AE is fatigue, nausea, vomiting, right upper quadrant pain or tenderness, fever, rash or eosinophilia. In some embodiments, the AE is a severe AE. In some embodiments, the severe AE is syncope or dyspnea. In some embodiments, the severe AE is an AE that is life-threatening or results in death.

In some embodiments, the AE is related to a drug-induced liver injury (DILI). In some embodiments, upon administering a dose DILI is indicated by an increase in one or more serum liver analytes. In some embodiments, the serum liver analyte is ALT and/or AST. In some embodiments, DILI is indicated when one or more of the following conditions are met: (1) ALT≥5×ULN; (2) ALT≥3×ULN, total bilirubin >2×ULN, and there is no or minimal elevation of ALP level; and/or (3) ALP≥2×ULN when the source of increased ALP level is the liver. In some embodiments, if a patient is assessed to have a DILI, a dose is decreased to the previous (i.e., lower) dose level. In some embodiments, when one or more of (1) ALT or AST>8×ULN; (2) ALT or AST>5×ULN for more than 2 weeks; (3) ALT or AST>3×ULN with the appearance of fatigue, nausea, vomiting, right upper quadrant pain, or tenderness, fever, rash, and/or eosinophilia (>5%), a dose is decreased to the previous (i.e., lower) dose level.

Once a maximum tolerated dose (MTD) is achieved, or a dose of 1280 μg is achieved during a titration period, without the presence of one or more AEs, the patient is administered said dose during a maintenance period, i.e., titration is complete and the patient remains on the MTD during the maintenance period phase of the administration period.

The length of the administration period (e.g., a (i) titration period, (ii) a titration period+maintenance period, or (iii) maintenance period) in any given case may depend on the nature and severity of the PH being treated and how well a patient tolerates and responds to the therapy. The treatment methods provided herein are provided as a chronic therapy, and as such, a patient is on-therapy as long as the therapy is safe and effective. Accordingly, the administration period in one embodiment, continues until a patient dies. In another embodiment, the administration period is the length of time the treatment is effective.

In some embodiments, the administration period is about 6 months, about 7 months, about 8 months, about 9 months, about 10 months, about 11 months, about 1 year, about 2 years, about 3 years, about 4 years, about 5 years, about 6 years, about 7 years, about 8 years, about 9 years, about 10 years, about 15 years, about 20 years or about 30 years.

In some embodiments, the administration period is at least about 6 months, at least about 7 months, at least about 8 months, at least about 9 months, at least about 10 months, at least about 11 months, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, at least about 30 years or at least about 50 years.

In another embodiment, the administration period for the methods provided herein is at least about 6 months, at least about 7 months, at least about 8 months, at least about 9 months, at least about 10 months, at least about 11 months, at least about 1 year, at least about 2 years, at least about 3 years, at least about 4 years, at least about 5 years, at least about 6 years, at least about 7 years, at least about 8 years, at least about 9 years or at least about 10 years or at least about 20 years. The administration period, in another embodiment, is from about 30 days to about 2 years. In another embodiment, the administration period is from about 6 months to about 3 years, or from 6 months to about 4 years, or from about 6 months to about 5 years, or from about 6 months to about 6 years, or from about 6 months to about 7 years, or from about 6 months to about 8 years, or from about 1 year to about 10 years, or from about 2 years to about 10 years, or from about 6 months to about 20 years, or from about 5 years to about 20 years, or from about 10 years to about 30 years.

In one embodiment, the administration period is at least about 1 year.

In one embodiment, the administration period is at least about 5 years.

In one embodiment, the administration period is from about 1 year to about 15 years. In another embodiment, the administration period is from about 5 years to about 15 years. In yet another embodiment, the administration period is from about 10 years to about 20 years. In even another embodiment, the administration period is from about 1 year to about 20 years.

In one embodiment, the administration period is from about 5 years to about 50 years. In another embodiment, the administration period is from about 5 years to about 40 years. In yet another embodiment, the administration period is from about 10 years to about 50 years. In even another embodiment, the administration period is from about 15 years to about 50 years.

In one embodiment, each dosing session comprises 1 to 10 inhalations (puffs) from a DPI, for example 1 inhalation (1 puff), 2 inhalations (2 puffs), 3 inhalations (3 puffs), 4 inhalations (4 puffs), 5 inhalations (5 puffs), 6 inhalations (6 puffs), 7 inhalations (7 puffs), 8 inhalations (8 puffs), 9 inhalations (9 puffs), or 10 inhalation (10 puffs). As used herein, a “dosing session” refers to 1 to 10 inhalations (puffs) from a DPI as required to administer from about 80 μg to about 1300 μg of the compound of Formula (I) or (II), an enantiomer or diastereomer thereof, or a pharmaceutically acceptable salt thereof. The DPI, in one embodiment, is small and transportable by the patient. In one embodiment, the DPI is a single dose DPI. In a preferred embodiment, the compound of Formula (I) is treprostinil palmitil.

In order to achieve a particular dose, in one embodiment, more than one DPI capsule comprising the composition can be employed. For example, in the case of a 640 μg dose, two 320 μg DPI capsules can be used and in the case of a 1280 μg dose, four 320 μg DPI capsules can be used. Each capsule can be administered via 1 or 2 inhalations, for example.

The effective amount of the compound of Formula (I) or (II), an enantiomer, diastereomer, or a pharmaceutically acceptable salt thereof, may include a fixed dose of a compound of Formula (I) or (II), an enantiomer or diastereomer thereof, or a pharmaceutically acceptable salt thereof. The fixed dose, in one embodiment, is present in one or multiple DPI capsules. The fixed dose, in one embodiment, is a dose that is titrated (either up or down) from a prior dose. In another embodiment, the fixed dose is the same dose or substantially the same dose as a prior dose. The effective amount, in one embodiment, is the amount of the compound of Formula (I) or (II), an enantiomer or diastereomer thereof, or a pharmaceutically acceptable salt thereof, administered during each dosing session. In some embodiments, the amount “administered” refers to the amount of the compound of Formula (I) or (II), an enantiomer or diastereomer thereof, or a pharmaceutically acceptable salt thereof, in the capsule, or multiple capsules in the DPI, administered in a single dosing session. In some embodiments, the fixed dose ranges from about 80 μg to about 1280 μg of a compound of Formula (U) or (II), an enantiomer or diastereomer thereof, or a pharmaceutically acceptable salt thereof, e.g., about 80 μg, about 112.5 μg, about 160 μg, about 225 μg, about 240 μg, about 320 μg, about 400 μg, about 450 μg, about 480 μg, about 640 μg, about 720 μg, about 800 μg, about 880 μg, about 960 μg, about 1040 μg, about 1120 μg, about 1200 μg, or about 1280 μg of the compound of Formula (II), an enantiomer or diastereomer thereof, or pharmaceutically acceptable salt thereof. For example, if the dry powder composition is administered once daily in a single dosing session, the effective amount can be considered to be the amount of the compound of Formula (I) or (II), an enantiomer or diastereomer thereof, or a pharmaceutically acceptable salt thereof, in the capsule or multiple capsules that is administered during the single dosing session. For example, in one embodiment, one or more capsules may be formulated with the dry powder composition wherein the one or more capsules have a total dose of about 80 μg, about 160 μg, about 240 μg, about 320 μg, about 400 μg, about 480 μg, about 560 μg, about 640 μg, about 720 μg, about 800 μg, about 880 μg, about 960 μg, about 1040 μg, about 1120 μg, about 1200 μg, or about 1280 μg of a compound of Formula (I) or (II), an enantiomer or diastereomer thereof, or a pharmaceutically acceptable salt thereof, and each of the aforementioned dosages may be an effective amount, and may also be referred to as the amount administered once daily in a single dosing session, during the administration period. As a further example, in one embodiment, the capsule comprises a dry powder composition comprising about 320 μg of a compound of Formula (II), an enantiomer or diastereomer thereof, or a pharmaceutically acceptable salt thereof, and, for purposes of this disclosure, the amount administered is 640 μg, even if takes 2 or more puffs from two capsules to administer the 640 μg. Similarly, in this example, the amount administered is 640 μg even if a residual amount of compound of Formula (II), an enantiomer or diastereomer thereof, or a pharmaceutically acceptable salt thereof remains in the DPI (e.g., if about 5%, 10%, 20%, 30%, 40%, or 50% remains in the DPI.)

The dose “administered” in a single dosing session also encompasses situations where the DPI is refiled or reloaded 1 or more times (e.g., by changing the capsules) in order to achieve the desired effective amount. In such situations, “administration” refers to the total dosage in the capsules which are administered in the dosing session. For example, to administer a dosage of 240 μg of a compound of Formula (II), an enantiomer or diastereomer thereof, or a pharmaceutically acceptable salt thereof, one 80 μg capsule and one 160 μg capsule may be used. The DPI may be filed with a first 80 μg capsule, and after emptying the cartridge in 1 or more puffs, a 160 μg capsule may be loaded in the DPI and emptied in 1 or more puffs. Both capsules are used in the same dosing session, and therefore the dose administered is 240 μg.

### Pulmonary Hypertension

The World Health Organization (WHO) has classified PH into five groups. Group 1 PH includes pulmonary arterial hypertension (PAH), idiopathic pulmonary arterial hypertension (IPAH), familial pulmonary arterial hypertension (FPAH), and pulmonary arterial hypertension associated with other diseases (APAI-L). For example, pulmonary arterial hypertension associated with collagen vascular disease (e.g., scleroderma), congenital shunts between the systemic and pulmonary circulation, portal hypertension and/or HIV infection are included in group 1 PH. Group 2 PH includes pulmonary hypertension associated with left heart disease, e.g., atrial or ventricular disease, or valvular disease (e.g., mitral stenosis). WHO group 3 pulmonary hypertension is characterized as pulmonary hypertension associated with lung diseases, e.g., chronic obstructive pulmonary disease (COPD), interstitial lung disease (ILD), and/or hypoxemia. Group 4 pulmonary hypertension is pulmonary hypertension due to chronic thrombotic and/or embolic disease. Group 4 PH is also referred to as chronic thromboembolic pulmonary hypertension. Group 4 PH patients experience blocked or narrowed blood vessels due to blood clots. Group 5 PH is the “miscellaneous” category, and includes PH caused by blood disorders (e.g., polycythemia vera, essential thrombocythemia), systemic disorders (e.g., sarcoidosis, vasculitis) and/or metabolic disorders (e.g., thyroid disease, glycogen storage disease).

The methods provided herein can be used to treat group 1, group 2, group 3, group 4 or group 5 PH patients, as characterized by the WHO.

In one embodiment of the methods, the pulmonary hypertension treated is chronic thromboembolic pulmonary hypertension.

In one preferred embodiment, the pulmonary hypertension is group 1 PH, as characterized by the New York Heart Association (NYHA). In a further embodiment, the method provided herein is a method for treating treated is pulmonary arterial hypertension (PAH). In a further embodiment, the PAH is class I PAH, class II PAH, class III PAH, or class IV PAH, as characterized by NYHA.

In one embodiment, the PAH is class I PAH, as characterized by the NYHA.

In another embodiment, the PAH is class II PAH, as characterized by the NYHA.

In yet another embodiment, the PAH is class III PAH, as characterized by the NYHA.

In still another embodiment, the PAH is class IV PAH, as characterized by the NYHA.

In one embodiment, the pulmonary hypertension (PH) is portopulmonary hypertension (PPH). PPH is defined by the coexistence of portal and pulmonary hypertension. The diagnosis of portopulmonary hypertension is based on hemodynamic criteria: (1) portal hypertension and/or liver disease (clinical diagnosis-ascites/varices/splenomegaly), (2) mean pulmonary artery pressure >25 mmHg at rest, (3) pulmonary vascular resistance >240 dynes s/cm⁵, (4) pulmonary artery occlusion pressure <15 mmHg or transpulmonary gradient >12 mmHg. PPH is a serious complication of liver disease, and is present in 0.25 to 4% of patients suffering from cirrhosis. PPH is comorbid in an estimated 4-6% of those referred for a liver transplant.

In one preferred embodiment, the pulmonary hypertension is group 3 PH, as characterized by the WHO. In a further embodiment, the method provided herein is a method for treating PH associated with interstitial lung disease (PH-ILD).

In the methods for treating PH-ILD provided herein, the ILD may include one or more lung conditions. The one or more lung conditions comprise, in one embodiment, idiopathic pulmonary fibrosis (IPF), cryptogenic organizing pneumonia (COP), desquamative interstitial pneumonitis, nonspecific interstitial pneumonitis, hypersensitivity pneumonitis, acute interstitial pneumonitis, interstitial pneumonia (e.g., idiopathic interstitial pneumonia), connective tissue disease, sarcoidosis or asbestosis. In one embodiment, the ILD is connective tissue disease-associated interstitial lung disease (CTD-ILD). In another embodiment, the ILD is sarcoidosis. In yet another embodiment, the ILD is IPF. In even another embodiment, the ILD is an idiopathic interstitial pneumonia (IIP).

In one embodiment for treating PH-ILD provided herein, the ILD includes pulmonary fibrosis, e.g., idiopathic pulmonary fibrosis (IPF). Pulmonary fibrosis is a respiratory disease in which scars are formed in the lung tissues, leading to serious breathing problems. Scar formation, i.e., the accumulation of excess fibrous connective tissue, leads to thickening of the walls, and causes reduced oxygen supply in the blood. As a result, pulmonary fibrosis patients suffer from perpetual shortness of breath. In some patients the specific cause of the disease can be diagnosed, but in others the probable cause cannot be determined, a condition called IPF.

In some embodiments, treating comprises one or more of the following of the patient, during the administration period as compared to prior to the administration period (1) reducing the pulmonary vascular resistance index (PVRI), (2) reducing the mean pulmonary artery pressure, (3) increasing the hypoxemia score, (4) decreasing the oxygenation index of the patient, (5) improving the right heart function, and (6) improving the exercise capacity (e.g., as measured by the six-minute walk test (6MWT)).

6MWT is a validated method for measuring exercise capacity and assessment of pulmonary function, and performed according to the American Thoracic Society (ATS) guidelines. See American Thoracic Society. ATS Statement: Guidelines for the six minute walk test. Am J Respir Crit Care Med. 2002; 166(1):111-17, incorporated herein by reference in its entirety for all purposes. In one embodiment, the 6MWT is performed at approximately the same time on a day during the administration period as on a day prior to the administration period. In a further embodiment, the same equipment is used to perform the 6MWT. In still a further embodiment, the same person administers the 6MWT.

In one embodiment, the patient's distance walked in the 6MWT is increased during the administration period, as compared to prior to the administration period, by at least about 5 meters, at least about 10 meters, at least about 20 meters, at least about 30 meters, at least about 40 meters, or at least about 50 meters. In another embodiment, the patient's distance walked in the 6MWT is increased during the administration period, as compared to prior to the administration period, by from about 5 meters to about 60 meters, by from about 5 meters to about 50 meters, by from about 10 meters to about 50 meters, by from about 15 meters to about 50 meters, or by from about 20 meters to about 40 meters. In yet another embodiment, the patient's distance walked in the 6MWT is increased by at least about 30 meters, during the administration period, compared to prior to the administration period.

In one embodiment, the patient's distance walked in the 6MWT is increased during the administration period, as compared to prior to the administration period, by about 1%, by about 2%, by about 3%, by about 4%, by about 5%, by about 6%, by about 7%, by about 8%, by about 9%, by about 10%, by about 11%, by about 12%, by about 13%, by about 14%, by about 15%, by about 16%, by about 17%, by about 18%, by about 19%, by about 20%, by about 25%, by about 30%, by about 35%, by about 40%, by about 45%, by about 50%, by about 55%, by about 60%, by about 65%, by about 70%, by about 75%, by about 80%, by about 85%, or by about 90%. In another embodiment, the patient's distance walked in the 6MWT is increased during the administration period, as compared to prior to the administration period, by at least about 5%, by at least about 10%, by at least about 15%, by at least about 20%, by at least about 25%, by at least about 30%, by at least about 35%, by at least about 40%, by at least about 45%, or by at least about 50%. In another embodiment, the patient's distance walked in the 6MWT is increased during the administration period, as compared to prior to the administration period, by about 5% to about 50%, by about 5% to about 40%, by about 5% to about 30%, by about 5% to about 20%, by about 10% to about 50%, by about 15% to about 50%, by about 20% to about 50%, or by about 25% to about 50%.

In one embodiment for treating PH, treating comprises improving the quality of life of the patient during the administration period, compared to the quality of life of the patient prior to the administration period. The quality of life, in one embodiment, is measured by the Cambridge Pulmonary Hypertension Outcome Review (CAMPHOR) Questionnaire. McCabe et al. (2013). Chest. 2013; 144(2):522-30, incorporated by reference herein in its entirety for all purposes. The CAMPHOR Questionnaire is a pulmonary hypertension specific measure of health-related quality of life (QOL) consisting of 3 sections that evaluate a total of 65 items (25 relating to symptoms, 15 relating to activities, and 25 relating to QOL). The CAMPHOR scoring is negatively weighted therefore, a higher score indicates worse QOL and greater functional limitation. Symptom and QOL items are both scored out of 25 and activity items have 3 possible responses (score 0-2), giving a score out of 30. Each CAMPHOR assessment takes an average of 10 minutes. In one embodiment for treating PH, treating comprises decreasing the patient's CAMPHOR Questionnaire score during the administration period, compared to the CAMPHOR Questionnaire score prior to the administration period. The decrease, in one embodiment, is by from 1 to about 10, from 1 to about 9, from 1 to 8, from 1 to 7, from 1 to 6, from 1 to 5, from 1 to 4, from 1 to 3 or from 1 to 2.

In one embodiment of a method for treating PH, the method comprises increasing the patient's saturation of peripheral capillary oxygenation (SpO₂) at rest assessed by pulse oximetry during the administration period, compared to the patient's SpO₂ at rest prior to the administration period.

Oxygen saturation is an indication of how much hemoglobin in the blood is bound to oxygen, and is typically provided as a percentage of oxyhemoglobin to the total hemoglobin. SpO₂ is an indication of oxygen saturation in the peripheral capillaries. Exemplary methods to measure SpO₂ include, but are not limited to, pulse oximetry using a pulse oximeter. In one embodiment of a method for treating PH provided herein, the method comprises increasing the patient's SpO₂ at rest during the administration period, as compared to prior to the administration period, by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or by at least about 90%. In another embodiment, the method for treating PH comprises increasing the patient's SpO₂ at rest during the administration period, as compared to prior to the administration period, by about 5% to about 50%, by about 5% to about 40%, by about 5% to about 30%, by about 5% to about 20%, by about 10% to about 50%, by about 15% to about 50%, by about 20% to about 50%, or by about 25% to about 50%.

In one embodiment, the method for treating PH provided herein comprises improving the lung function of the patient during the administration period, as compared to the lung function of the patient prior to the administration period. The improvement in lung function in one embodiment, is measured by spirometry.

Improving the lung function of the patient, in one embodiment, comprises increasing the patient's forced vital capacity (FVC), increasing the patient's percent predicted forced vital capacity (ppFVC), increasing the patient's forced expiratory volume in 1 second (FEV₁), increasing the patient's percent predicted forced expiratory volume in one second (ppFEV₁), increasing the patient's forced expiratory flow between 25% and 75% of FVC (FEF_{(25-75%)}), increasing the patient's total lung capacity (TLC), or increasing the patient's lung diffusion capacity for carbon monoxide (DLCO), during the administration period, as compared to the respective value prior to the administration period.

The assessment of lung function, e.g., via FVC, ppFVC, FEV₁, ppFEV₁, FEF_{(25-75%)}, TLC, or DLCO measurement, in one embodiment, comprises comparing the lung function in the patient prior to the administration period, e.g., immediately prior to treatment, to a time point during the administration period the administration period, or to an average of measurements taken during the administration period.

As provided herein, in one embodiment, the method for treating PH comprises improving the lung function in the patient during the administration period, as compared to the respective value prior to the administration period, wherein the lung function is measured by spirometry. Spirometry is a physiological test that measures how an individual inhales or exhales volumes of air. The primary signal measured in spirometry may be volume or flow. For the methods described herein, pulmonary function test (PFT) by spirometry (e.g., FEV₁, FVC, FEF_{(25-75%)}, and TLC) is performed per the American Thoracic Society (ATS)/European Respiratory Society (ERS) criteria, e.g., as set forth by Miller et al. (Miller et al., “Standardization of Spirometry,” Eur. Respir. J. 26:319-38 (2005), incorporated by reference herein in its entirety for all purposes). DLCO can be measured using techniques described by Modi P, Cascella M, “Diffusing Capacity Of The Lungs For Carbon Monoxide,” [Updated 2021 Mar. 24]. In: StatPearls [Internet]. Treasure Island (FL): StatPearls Publishing; 2021 January-. Available from: www.ncbi.nlm.nih.gov/books/NBK556149/; Graham et al., “2017 ERS/ATS standards for single-breath carbon monoxide uptake in the lung,” European Respiratory Journal 49:1600016 (2017); each of which is incorporated herein by reference in its entirety for all purposes.

In one embodiment, the spirometer is capable of accumulating volume for greater than or equal to 15 seconds, e.g., ≥20 seconds, ≥25 seconds, ≥30 seconds, ≥35 seconds. The spirometer in one embodiment can measure volumes of ≥8 L (BiTPS) with an accuracy of at least ±3% of reading or ±0.050 L, whichever is greater, with flows between 0 and 14 L·s⁻¹. In one embodiment, the total resistance to airflow of the spirometer at 14 L·s⁻¹ is <1.5 cmH₂O·L⁻¹·s⁻¹ (0.15 kPa?L⁻¹·s⁻¹). In one embodiment, the total resistance of the spirometer is measured with any tubing, valves, pre-filter, etc. included that may be inserted between the patient and the spirometer. With respect to devices that exhibit changes in resistance due to water vapor condensation, in one embodiment, spirometer accuracy requirements are met under BTPS (body temperature, ambient pressure, saturated with water vapor) conditions for up to eight successive FVC maneuvers performed in a 10-min period without inspiration from the instrument.

With respect to the forced expiratory maneuvers described herein, in one embodiment, the range and accuracy recommendations as set forth in Table 6 of Miller et al., are met (Miller et al., “Standardization of Spirometry,” Eur. Respir. J. 26:319-38 (2005), incorporated by reference herein in its entirety for all purposes).

In one embodiment, improving lung function comprises improving the forced vital capacity (FVC) of the patient, i.e., the maximal volume of air exhaled with maximally forced effort from a maximal inspiration, during the administration period, as compared to the FVC prior to the administration period. The FVC is expressed in liters at body temperature and ambient pressure saturated with water vapor (BTPS). In another embodiment, the improvement in lung function is an improvement in the percent predicted forced vital capacity (ppFVC).

“Forced vital capacity” (FVC) denotes the volume of gas which is exhaled during a forced expiration starting from a position of full inspiration and ending at complete expiration and is one measure of treatment efficacy. FVC may be expressed as a percentage of the predicted FVC (i.e., ppFVC) obtained from a normal population, based on the patient's age, height, gender, and sometimes weight and race. In one embodiment of a method for treating PH, improving the patient's lung function comprises increasing the patient's FVC or ppFVC during the administration period, compared to the patient's corresponding FVC or ppFVC prior to the administration period. The increase in FVC or ppFVC, in one embodiment, is an increase of at least about 5%, at least about 10%, at least about 15%, at least about 20%, at least about 25%, at least about 30%, at least about 35%, at least about 40%, at least about 45%, or at least about 50%. In another embodiment, the increase in FVC or ppFVC is an increase of from about 1% to about 20%, from about 1% to about 15%, from about 1% to about 10%, from about 1% to about 5%, from about 5% to about 50%, from about 5% to about 40%, from about 5% to about 30%, from about 5% to about 20%, from about 10% to about 50%, from about 15% to about 50%, from about 20% to about 50%, or from about 25% to about 50%. In one embodiment, increasing FVC or ppFVC is increasing pre-bronchodilator FVC or ppFVC. In another embodiment, increasing FVC or ppFVC is increasing post-bronchodilator FVC or ppFVC.

In one embodiment, the patient's ppFVC is 80% or less prior to the administration period. In a further embodiment, the patient's ppFVC is 70% or less prior to the administration period. In a further embodiment, the patient's ppFVC is 60% or less prior to the administration period. In a further embodiment, the patient's ppFVC is 50% or less prior to the administration period. In another embodiment, the patient's ppFVC is from 30% to 80%, from 40% to 70%, or from 50% to 60%, prior to the administration period.

FVC maneuvers can be performed according to the procedures known to those of ordinary skill in the art. Briefly, the three distinct phases to the FVC maneuver are (1) maximal inspiration; (2) a “blast” of exhalation and (3) continued complete exhalation to the end of test (EOT). The maneuver can be carried out via the closed circuit method or open circuit method. In either instance, the patient inhales rapidly and completely with a pause of less than 1 second at total lung capacity (TLC). The patient then exhales maximally until no more air can be expelled while maintaining an upright posture. The exhalation begins with a “blast” of air from the lungs and then is encouraged to fully exhale. Enthusiastic coaching of the patient continues for a minimum of three maneuvers.

FEV is the volume of gas exhaled in a specified time (typically 1 second, i.e., FEV₁) from the start of the forced vital capacity maneuver (Quanjer et al. (1993). Eur. Respir. J. 6, Suppl. 16, pp. 5-40, incorporated by reference herein in its entirety for all purposes). FEV₁ may also be expressed as a percentage of the predicted FEV₁ (i.e., ppFEV₁) obtained from a normal population, based on the patient's gender, height, and age, and sometimes race and weight.

In one embodiment, improving the lung function of the patient comprises increasing the patient's FEV₁ or ppFEV₁ during the administration period, compared to the patient's corresponding FEV₁ or ppFEV₁ prior to the administration period. The increase in FEV₁ or ppFEV₁, in one embodiment, is an increase of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, or about 90%. In another embodiment, the increase in FEV₁ or ppFEV₁ is an increase of about 5%, about 10%, about 15%, about 20%, about 25%, about 30%, about 35%, about 40%, about 45%, or about 50%. In another embodiment, increasing the FEV₁ or ppFEV₁ comprises increasing by at least about 5%, at least about 10%, at least about 15%, at least about 20%, at least about 25%, at least about 30%, at least about 35%, at least about 40%, at least about 45%, or at least about 50%. In another embodiment, increasing FEV₁ or ppFEV₁ is increasing of about 5% to about 50%, about 5% to about 40%, about 5% to about 30%, about 5% to about 20%, about 10% to about 50%, about 15% to about 50%, about 20% to about 50%, or about 25% to about 50%.

In one embodiment, increasing FEV₁ or ppFEV₁ is increasing in pre-bronchodilator FEV₁ or ppFEV₁. In another embodiment, increasing FEV₁ or ppFEV₁ is increasing post-bronchodilator FEV₁ or ppFEV₁.

In one embodiment, the patient's ppFEV₁ is 80% or less prior to the administration period. In a further embodiment, the patient's ppFEV₁ is 70% or less prior to the administration period. In a further embodiment, the patient's ppFEV₁ is 60% or less prior to the administration period. In a further embodiment, the patient's ppFEV₁ is 50% or less prior to the administration period. In another embodiment, the patient's pp FEV₁ is from 30% to 80%, from 40% to 70%, or from 50% to 60%, prior to the administration period.

In another embodiment, improving the lung function of the patient comprises increasing the patient's FEV₁ during the administration period, compared to prior to the administration period, by from about 25 mL to about 500 mL, from about 25 mL to about 400 mL, from about 25 mL to about 300 mL, from about 25 mL to about 250 mL, from about 25 mL to about 200 mL, or from about 50 mL to about 200 mL, as compared to the patient's FEV₁ prior to the administration period. In one embodiment, increasing FEV₁ is increasing pre-bronchodilator FEV₁. In another embodiment, increasing FEV₁ is increasing post-bronchodilator FEV₁.

In one embodiment, improving the lung function of the patient comprises increasing the mean forced expiratory flow between 25% and 75% of FVC (FEF_{(25-75%)}) (also referred to as the maximum mid-expiratory flow) of the patient during the administration period, as compared to the patient's FEF_{(25-75%)} prior to the administration period. The FEF_{(25-75%)} measurement is dependent on the validity of the FVC measurement and the level of expiratory effort. The FEF_{(25-75%)} index is taken from the blow with the largest sum of FEV₁ and FVC.

In one embodiment, increasing the patient's FEF_{(25-75%)} during the administration period comprises increasing by at least about 1%, by at least about 5%, by at least about 10%, by at least about 15%, by at least about 20%, by at least about 25%, by at least about 30%, by at least about 35%, by at least about 40%, by at least about 45%, or by at least about 50%. In another embodiment, increasing the patient's FEF_{(25-75%)} during the administration period comprises increasing by about 5% to about 50%, by about 5% to about 40%, by about 5% to about 30%, by about 5% to about 20%, by about 10% to about 50%, by about 15% to about 50%, by about 20% to about 50%, or by about 25% to about 50%. In one embodiment, increasing FEF_{(25-75%)} is increasing pre-bronchodilator FEF_{(25-75%)}. In another embodiment, increasing FEF_{(25-75%)} is increasing post-bronchodilator FEF_{(25-75%)}.

Total lung capacity (TLC) is the sum of the vital capacity and residual volume that represents the total volume of air that can be contained in the lung. The total lung capacity (TLC) is divided into four volumes. The tidal volume (VT) is the volume inhaled or exhaled in normal quiet breathing. The inspiratory reserve volume (IRV) is the maximum volume that can be inhaled following a normal quiet inhalation. The expiratory reserve volume (ERV) is the maximum volume that can be exhaled following a normal quiet exhalation. The residual volume (RV) is the volume remaining in the lungs following a maximal exhalation. The vital capacity (VC) is the maximum volume that can be exhaled following a maximal inhalation; VC=IRV+V_{T}+ERV. In one embodiment, improving the lung function of the patient comprises increasing the patient's total lung capacity (TLC) during the administration period, compared to the patient's TLC prior to the administration period. In one embodiment, increasing is by at least about 1%, at least about 2%, at least about 5%, at least about 10%, at least about 15%, at least about 20%, at least about 25%, at least about 30%, at least about 35%, at least about 40%, at least about 45%, or by at least about 50%. In another embodiment, increasing is by from about 1% to about 50%, by from about 5% to about 50%, by from about 5% to about 40%, by from about 5% to about 30%, by from about 5% to about 20%, by from about 10% to about 50%, by from about 15% to about 50%, by from about 20% to about 50%, or by from about 25% to about 50%.

Also known as the transfer factor, lung diffusion capacity for carbon monoxide (DLCO) is a measurement to assess the lungs' ability to transfer gas from inspired air to the bloodstream. Carbon monoxide (CO) has a high affinity for hemoglobin, and it follows the same pathway as that of oxygen to finally bind with hemoglobin. Inhaled CO is used for this test due to its high affinity for hemoglobin (200 to 250 times that of oxygen). As anemia can reduce DLCO, DLCO may be adjusted for hemoglobin values. DLCO may also need to be adjusted for several other factors, such as carboxyhemoglobin, FiO. See Modi P, Cascella M, “Diffusing Capacity Of The Lungs For Carbon Monoxide,” [Updated 2021 Mar. 24]. In: StatPearls [Internet]. Treasure Island (FL): StatPearls Publishing; 2021 January, incorporated herein by reference in its entirety for all purposes. In one embodiment, improving the lung function of the patient comprises increasing the patient's DLCO during the administration period, compared to the patient's DLCO prior to the administration period. In one embodiment, DLCO is adjusted for hemoglobin level, i.e., improving the lung function of the patient comprises increasing the patient's DLCO adjusted for hemoglobin during the administration period compared to the patient's DLCO adjusted for hemoglobin prior to the administration period. In another embodiment, improving the lung function of the patient comprises increasing the patient's DLCO percent (DLCO %) predicted during the administration period compared to the patient's DLCO % predicted prior to the administration period. Predicted normal DLCO values may be calculated according to the equation established by Crapo et al., Am Rev Respir Dis. 123(2):185-9 (1981), or according to the equation established by Miller et al., Am Rev Respir Dis. 127(3):270-7 (1983), each of which is incorporated by reference in its entirety for all purposes. In a further embodiment, the patient's DLCO % predicted is adjusted for hemoglobin.

In one embodiment, improving lung function comprises increasing the patient's DLCO or DLCO % predicted by at least about 1%, at least about 5%, at least about 10%, at least about 15%, at least about 20%, at least about 25%, at least about 30%, at least about 35%, at least about 40%, at least about 45%, or by at least about 50%. In another embodiment, improving lung function comprises increasing the patient's DLCO or DLCO % predicted by from about 5% to about 50%, by from about 5% to about 40%, by from about 5% to about 30%, by from about 5% to about 20%, by from about 10% to about 50%, by from about 15% to about 50%, by from about 20% to about 50%, or by from about 25% to about 50%. In a further embodiment, the patient's DLCO or DLCO % predicted is adjusted for hemoglobin.

In one embodiment, the patient's DLCO % predicted is 80% or less, 70% or less, 60% or less, or 50% or less, prior to the administration period. In a further embodiment, the patient's DLCO % predicted is adjusted for hemoglobin. In another embodiment, the patient's DLCO % predicted is from 30% to 80%, from 40% to 70%, or from 50% to 60%, prior to the administration period. In a further embodiment, the patient's DLCO % predicted is adjusted for hemoglobin.

In one embodiment of a method for treating PH provided herein, the method comprises increasing the length of time to clinical worsening, as compared to an untreated PH patient, or a PH patient not treated with a compound of Formula (I) or (II), wherein the clinical worsening is one selected from the group consisting of death, hospitalization due to a respiratory indication (e.g., dyspnea, and/or deterioration of lung function indicated by reductions in FVC, DL CO, and/or SpO₂), 10% or greater decline in percent predicted FVC (ppFVC) relative to the patient's ppFVC prior to the administration period on two consecutive occasions 4-14 weeks apart, lung transplantation, and 15% or greater decrease in distance walked in a 6-minute walk test (6MWT) relative to the patient's distance walked in a 6MWT prior to the administration period on two consecutive occasions at least 24 hours apart.

In one embodiment, the length of time to clinical worsening is increased by about 1 day, about 3 days, about 1 week, about 2 weeks, about 3 weeks, about 4 weeks, about 5 weeks, or about 6 weeks. In another embodiment, the length of time to clinical worsening is increased by at least about 1 day, at least about 3 days, at least about 1 week, at least about 2 weeks, at least about 3 weeks, at least about 4 weeks, at least about 5 weeks, or at least about 6 weeks. In another embodiment, the length of time to clinical worsening is increased about 20 days to about 100 days, about 30 days to about 100 days, about 20 days to about 75 days, about 20 days to about 50 days, or about 20 days to about 40 days. In another embodiment, the length of time to clinical worsening is increased at least 1 month, e.g., about 1 month to about 6 months, about 1 month to about 4 months, or about 1 month to about 3 months.

In one embodiment, a method for treating PH provided herein comprises increasing the patient's lung lobar volume and/or airway volume assessed by computerized tomography (CT) during the administration period, compared to the patient's lung lobar volume and/or airway volume prior to the administration period. CT may be performed via chest CT scan during a breathing cycle to generate CT images at functional residual capacity (FRC) and/or total lung capacity (TLC). In one embodiment, the lung lobar volume is the volume of the lung lobar structure of the patient's respiratory system at TLC or FRC, and the airway volume is the volume of the airway structure of the patient's respiratory system at TLC or FRC.

In one embodiment, increasing the patient's lung lobar volume and/or airway volume comprises increasing by at least about 1%, at least about 5%, at least about 10%, at least about 15%, at least about 20%, at least about 25%, at least about 30%, at least about 35%, at least about 40%, at least about 45%, or by at least about 50%. In another embodiment, the patient's lung lobar volume and/or airway volume is increased by from about 5% to about 50%, by from about 5% to about 40%, by from about 5% to about 30%, by from about 5% to about 20%, by from about 10% to about 50%, by from about 15% to about 50%, by from about 20% to about 50%, or by from about 25% to about 50%.

In some embodiments, the method for initiating treatment of pulmonary hypertension comprises administering to the subject an effective amount of a dry powder composition. The dry powder composition comprises a compound of Formula (I) or (II) disclosed herein, or a pharmaceutically acceptable salt thereof. The administering comprises (i) aerosolizing the dry powder composition via a DPI to provide an aerosolized dry powder composition, and (ii) administering the aerosolized dry powder composition to the lungs of the patient via inhalation by the DPI.

In one embodiment of a method for treating pulmonary hypertension in a patient in need thereof, wherein the treating comprises remodeling the pulmonary vasculature of the patient. In some embodiments, the treating comprises administering via a DPI to the patient, once daily in a single dosing session during an administration period, an effective amount of a compound of Formula (I), or an enantiomer, diastereomer, or a pharmaceutically acceptable salt thereof, wherein R¹ is tetradecyl, pentadecyl, hexadecyl, heptadecyl, or octadecyl. In some embodiments, the R¹ is hexadecyl.

In some embodiments, the present disclosure provides a method of remodeling pulmonary vasculature in a patient with PH-ILD, comprising administering via a DPI to the patient, once daily in a single dosing session during an administration period, an effective amount of a compound of Formula (I), or an enantiomer, diastereomer, or a pharmaceutically acceptable salt thereof, wherein R¹ is tetradecyl, pentadecyl, hexadecyl, heptadecyl, or octadecyl. In some embodiments, the R¹ is hexadecyl.

In some embodiments, the present disclosure provides a method of remodeling pulmonary vasculature in a patient with PH-ILD, comprising administering via a DPI to the patient, once daily in a single dosing session during an administration period, an effective amount of a treprostinil prodrug selected from one of the prodrugs set forth in U.S. Pat. Nos. 12,173,021, 12,168,071, U.S. Patent Application Publication No. 2023/0255922, U.S. Patent Application Publication No. 2024/0051979, U.S. Patent Application Publication No. 2024/0058287, or U.S. Patent Application Publication No. 2024/0130989, the contents of which are incorporated by reference herein in their entireties for all purposes. In a further embodiment, the dose of the compound is titrated to a maximum titrated dose during the administration period.

In some embodiments, remodeling pulmonary vasculature comprises increasing the total blood volume in small pulmonary arteries during the administration period compared to the total blood volume in small pulmonary arteries prior to the administration period. Increasing the total blood volume in small pulmonary arteries, in some embodiments, occurs during the titration period of an administration period. In another embodiment, increasing the total blood volume in small pulmonary arteries occurs during a maintenance period of an administration period.

In some embodiments, increasing the total blood volume in small pulmonary arteries comprises increasing by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 30%, at least about 35%, at least about 40%, or at least about 50% during the administration period compared to the total blood volume in small pulmonary arteries prior to the administration period. In some embodiments, increasing the total blood volume in small pulmonary arteries comprises increasing by about 10% to about 50% during the administration period compared to the total blood volume in small pulmonary arteries prior to the administration period. In some embodiments, increasing the total blood volume in small pulmonary arteries comprises increasing by about 10% to about 40% during the administration period compared to the total blood volume in small pulmonary arteries prior to the administration period. In some embodiments, increasing the total blood volume in small pulmonary arteries comprises increasing by about 10% to about 30% during the administration period compared to the total blood volume in small pulmonary arteries prior to the administration period. In some embodiments, increasing the total blood volume in small pulmonary arteries comprises increasing by about 5% to about 15% during the administration period compared to the total blood volume in small pulmonary arteries prior to the administration period. In some embodiments, increasing the total blood volume in small pulmonary arteries comprises increasing by about 5% to about 10% during the administration period compared to the total blood volume in small pulmonary arteries prior to the administration period. In some embodiments, increasing the total blood volume in small pulmonary arteries comprises increasing by about 10% to about 15% during the administration period compared to the total blood volume in small pulmonary arteries prior to the administration period. In some embodiments, increasing the total blood volume in small pulmonary arteries comprises increasing by about 10% to about 20% during the administration period compared to the total blood volume in small pulmonary arteries prior to the administration period. In some embodiments, increasing the total blood volume in small pulmonary arteries comprises increasing by about 15% to about 20% during the administration period compared to the total blood volume in small pulmonary arteries prior to the administration period. In some embodiments, increasing the total blood volume in small pulmonary arteries comprises increasing by about 14.0% during the administration period compared to the total blood volume in small pulmonary arteries prior to the administration period.

In some embodiments, the total blood volume in small pulmonary arteries is at least about 10 mL, at least about 11 mL, at least about 12 mL, at least about 13 mL, at least about 14 mL, at least about 15 mL, at least about 16 mL, at least about 17 mL, at least about 18 mL, at least about 19 mL, at least about 20 mL, at least about 21 mL, at least about 22 mL, at least about 23 mL, at least about 24 mL, at least about 25 mL, at least about 26 mL, at least about 27 mL, at least about 28 mL, at least about 29 mL, at least about 30 mL, at least about 31 mL, at least about 32 mL, at least about 33 mL, at least about 34 mL, at least about 35 mL, at least about 36 mL, at least about 37 mL, at least about 38 mL, at least about 39 mL, or at least about 40 mL during the administration period. In some embodiments, the total blood volume small pulmonary arteries is about 10 mL to about 40 mL during the administration period. In some embodiments, the total blood volume in small pulmonary arteries is about 15 mL to about 35 mL during the administration period. In some embodiments, the total blood volume in small pulmonary arteries is about 15 mL to about 30 mL during the administration period. In some embodiments, the total blood volume in small pulmonary arteries is about 15 mL to about 25 mL during the administration period. In some embodiments, the total blood volume in small pulmonary arteries is about 20 mL to about 25 mL during the administration period. In some embodiments, the total blood volume in small pulmonary arteries is about 21.4 mL during the administration period.

In some embodiments, remodeling pulmonary vasculature comprises increasing the proportion of total blood volume in small pulmonary arteries during the administration period compared to the proportion of total blood volume in small pulmonary arteries prior to the administration period. In some embodiments, increasing the proportion of total blood volume in small pulmonary arteries comprises increasing by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 30%, at least about 35%, at least about 40%, or at least about 50% during the administration period compared to the proportion of total blood volume in small pulmonary arteries prior to the administration period. In some embodiments, increasing the proportion of total blood volume in small pulmonary arteries comprises increasing by about 5% to about 15% during the administration period compared to the proportion of total blood volume in small pulmonary arteries prior to the administration period. In some embodiments, increasing the proportion of total blood volume in small pulmonary arteries comprises increasing by about 5% to about 10% during the administration period compared to the proportion of total blood volume in small pulmonary arteries prior to the administration period. In some embodiments, increasing the proportion of total blood volume in small pulmonary arteries comprises increasing by about 10% to about 15% during the administration period compared to the proportion of total blood volume in small pulmonary arteries prior to the administration period. In some embodiments, increasing the proportion of total blood volume in small pulmonary arteries comprises increasing by about 10% to about 20% during the administration period compared to the proportion of total blood volume in small pulmonary arteries prior to the administration period. In some embodiments, increasing the proportion of total blood volume in small pulmonary arteries comprises increasing by about 5% to about 20% during the administration period compared to the proportion of total blood volume in small pulmonary arteries prior to the administration period. In some embodiments, increasing the proportion of total blood volume in small pulmonary arteries comprises increasing by about 8.4% during the administration period compared to the proportion of total blood volume in small pulmonary arteries prior to the administration period.

In some embodiments, the proportion of total blood volume in small pulmonary arteries is at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 45%, or at least about 50% during the administration period. In some embodiments, the proportion of total blood volume in small pulmonary arteries is about 10% to about 50% during the administration period. In some embodiments, the proportion of total blood volume in small pulmonary arteries is about 20% to about 40% during the administration period.

In some embodiments, the proportion of total blood volume in small pulmonary arteries is about 20% to about 30% during the administration period. In some embodiments, the proportion of total blood volume in small pulmonary arteries is about 25% to about 30% during the administration period. In some embodiments, the proportion of total blood volume in small pulmonary arteries is about 26% during the administration period.

In some embodiments, remodeling pulmonary vasculature comprises increasing the treatment effect of the proportion of total blood volume in small pulmonary arteries upon administration of the compound, e.g., the compound of Formula (I) or (II), or an enantiomer, diastereomer, or a pharmaceutically acceptable salt thereof to a subject with PH-ILD, compared to an untreated subject with PH-ILD (e.g., a subject with PH-ILD administered a placebo control). In some embodiments, increasing the treatment effect comprises increasing by at least about 0.5%, at least about 1.0%, at least about 1.5%, at least about 2%, at least about 2.5%, at least about 3%, at least about 2.5%, at least about 4%, at least about 2.5%, at least about 5%, at least about 2.5%, at least about 6%, at least about 6.5%, at least about 7%, at least about 7.5%, at least about 8%, at least about 8.5%, at least about 9%, at least about 9.5%, at least about 10%, at least about 10.5%, at least about 11%, at least about 11.5%, at least about 12%, at least about 12.5%, at least about 13%, at least about 13.5%, at least about 14%, at least about 14.5%, or at least about 15% upon administration of the compound of Formula (I), or an enantiomer, diastereomer, or a pharmaceutically acceptable salt thereof to a subject with PH-ILD, compared to an untreated subject with PH-ILD. In some embodiments, increasing the treatment effect comprises increasing by about 1% to about 10% upon administration of the compound of Formula (I), or an enantiomer, diastereomer, or a pharmaceutically acceptable salt thereof to a subject with PH-ILD, compared to an untreated subject. In some embodiments, increasing the treatment effect comprises increasing by about 1% to about 5% upon administration of the compound, e.g., the compound of Formula (I) or (II), or an enantiomer, diastereomer, or a pharmaceutically acceptable salt thereof to a subject with PH-ILD, compared to an untreated subject with PH-ILD. In some embodiments, increasing the treatment effect comprises increasing by about 2.5% to about 7.5% upon administration of the compound of Formula (I), or an enantiomer, diastereomer, or a pharmaceutically acceptable salt thereof to a subject with PH-ILD, compared to an untreated subject with PH-ILD. In some embodiments, increasing the treatment effect comprises increasing by about 2.5% to about 5.0% upon administration of the compound of Formula (I), or an enantiomer, diastereomer, or a pharmaceutically acceptable salt thereof to a subject with PH-ILD, compared to an untreated subject with PH-ILD. In some embodiments, increasing the treatment effect comprises increasing by about 3.8% upon administration of the compound, e.g., the compound of Formula (I) or (II), or an enantiomer, diastereomer, or a pharmaceutically acceptable salt thereof to a subject with PH-ILD, compared to an untreated subject with PH-ILD. In some embodiments, the treatment effect is adjusted for fibrosis.

In some embodiments, remodeling pulmonary vasculature comprises increasing the proportion of total arterial volume in small pulmonary arteries during the administration period compared to the proportion of total arterial volume in small pulmonary arteries prior to the administration period. In some embodiments, increasing the proportion of total arterial volume in small pulmonary arteries comprises increasing by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 30%, at least about 35%, at least about 40%, or at least about 50% during the administration period compared to the proportion of total arterial volume in small pulmonary arteries prior to the administration period. In some embodiments, increasing the proportion of total arterial volume in small pulmonary arteries comprises increasing by about 5% to about 10% during the administration period compared to the proportion of total arterial volume in small pulmonary arteries prior to the administration period. In some embodiments, increasing the proportion of total arterial volume in small pulmonary arteries comprises increasing by about 5% to about 15% during the administration period compared to the proportion of total arterial volume in small pulmonary arteries prior to the administration period. In some embodiments, increasing the proportion of total arterial volume in small pulmonary arteries comprises increasing by about 10% to about 15% during the administration period compared to the proportion of total arterial volume in small pulmonary arteries prior to the administration period. In some embodiments, increasing the proportion of total arterial volume in small pulmonary arteries comprises increasing by about 5% to about 20% during the administration period compared to the proportion of total arterial volume in small pulmonary arteries prior to the administration period. In some embodiments, increasing the proportion of total arterial volume in small pulmonary arteries comprises increasing by about 8.0% during the administration period compared to the proportion of total arterial volume in small pulmonary arteries prior to the administration period.

In some embodiments, the proportion of total arterial volume in small pulmonary arteries is at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, or at least about 60% during the administration period. In some embodiments, the proportion of total arterial volume in small pulmonary arteries is about 20% to about 80% during the administration period. In some embodiments, the proportion of total arterial volume in small pulmonary arteries is about 20% to about 60% during the administration period. In some embodiments, the proportion of total arterial volume in small pulmonary arteries is about 30% to about 50% during the administration period. In some embodiments, the proportion of total arterial volume in small pulmonary arteries is about 40% to about 50% during the administration period. In some embodiments, the proportion of total arterial volume in small pulmonary arteries is about 35% to about 55% during the administration period. In some embodiments, the proportion of total arterial volume in small pulmonary arteries is about 47.8% during the administration period.

In some embodiments, remodeling pulmonary vasculature comprises increasing the treatment effect of the proportion of total arterial volume in small pulmonary arteries upon administration of the compound, e.g., the compound of Formula (I) or (II), or an enantiomer, diastereomer, or a pharmaceutically acceptable salt thereof to a subject with PH-ILD, compared to an untreated subject with PH-ILD (i.e., a subject with PH-ILD administered a placebo control). In some embodiments, increasing the treatment effect comprises increasing by at least about 0.5%, at least about 1.0%, at least about 1.5%, at least about 2%, at least about 2.5%, at least about 3%, at least about 2.5%, at least about 4%, at least about 2.5%, at least about 5%, at least about 2.5%, at least about 6%, at least about 6.5%, at least about 7%, at least about 7.5%, at least about 8%, at least about 8.5%, at least about 9%, at least about 9.5%, at least about 10%, at least about 10.5%, at least about 11%, at least about 11.5%, at least about 12%, at least about 12.5%, at least about 13%, at least about 13.5%, at least about 14%, at least about 14.5%, or at least about 15% upon administration of the compound, e.g., the compound of Formula (I), or an enantiomer, diastereomer, or a pharmaceutically acceptable salt thereof to a subject with PH-ILD, compared to an untreated subject with PH-ILD. In some embodiments, increasing the treatment effect comprises increasing by about 1% to about 15% upon administration of the compound of the disclosure, e.g., the compound of Formula (I) or (II), or an enantiomer, diastereomer, or a pharmaceutically acceptable salt thereof to a subject with PH-ILD, compared to an untreated subject with PH-ILD. In some embodiments, increasing the treatment effect comprises increasing by about 1% to about 10% upon administration of the compound of Formula (I), or an enantiomer, diastereomer, or a pharmaceutically acceptable salt thereof to a subject with PH-ILD, compared to an untreated subject with PH-ILD. In some embodiments, increasing the treatment effect comprises increasing by about 2.5% to about 7.5% upon administration of the compound of the disclosure, e.g., the compound of Formula (I) or (II), or an enantiomer, diastereomer, or a pharmaceutically acceptable salt thereof to a subject with PH-ILD, compared to an untreated subject with PH-ILD. In some embodiments, increasing the treatment effect comprises increasing by about 5.0% to about 7.5% upon administration of the compound of the disclosure, e.g., the compound of Formula (I) or (II), or an enantiomer, diastereomer, or a pharmaceutically acceptable salt thereof to a subject with PH-ILD, compared to an untreated subject with PH-ILD. In some embodiments, increasing the treatment effect comprises increasing by about 6.4% upon administration of the compound of the disclosure, e.g., the compound of Formula (I) or (II), or an enantiomer, diastereomer, or a pharmaceutically acceptable salt thereof to a subject with PH-ILD, compared to a subject with PH-ILD. In some embodiments, the treatment effect is adjusted for fibrosis.

In some embodiments, remodeling pulmonary vasculature comprises decreasing the total blood volume in large pulmonary arteries during the administration period compared to the total blood volume in large pulmonary arteries prior to the administration period. In some embodiments, decreasing the total blood volume in large pulmonary arteries comprises decreasing by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 30%, at least about 35%, at least about 40%, at least about 45%, at least about 50%, at least about 55%, at least about 60%, at least about 65%, at least about 70%, or at least about 75% during the administration period compared to the total blood volume in large pulmonary arteries prior to the administration period. In some embodiments, decreasing the total blood volume in large pulmonary arteries comprises decreasing by about 5% to about 50% during the administration period compared to the total blood volume in large pulmonary arteries prior to the administration period. In some embodiments, decreasing the total blood volume in large pulmonary arteries comprises decreasing by about 5% to about 25% during the administration period compared to the total blood volume in large pulmonary arteries prior to the administration period. In some embodiments, decreasing the total blood volume in large pulmonary arteries comprises decreasing by about 5% to about 20% during the administration period compared to the total blood volume in large pulmonary arteries prior to the administration period. In some embodiments, decreasing the total blood volume in large pulmonary arteries comprises decreasing by about 5% to about 15% during the administration period compared to the total blood volume in large pulmonary arteries prior to the administration period. In some embodiments, decreasing the total blood volume in large pulmonary arteries comprises decreasing by about 5% to about 10% during the administration period compared to the total blood volume in large pulmonary arteries prior to the administration period. In some embodiments, decreasing the total blood volume in large pulmonary arteries comprises decreasing by about 7.1% during the administration period compared to the total blood volume in large pulmonary arteries prior to the administration period.

In some embodiments, the total blood volume in large pulmonary arteries is at least about 1 mL, at least about 2 mL, at least about 3 mL, at least about 4 mL, at least about 5 mL, at least about 6 mL, at least about 7 mL, at least about 8 mL, at least about 9 mL, at least about 10 mL, at least about 11 mL, at least about 12 mL, at least about 13 mL, at least about 14 mL, at least about 15 mL, at least about 16 mL, at least about 17 mL, at least about 18 mL, at least about 19 mL, at least about 20 mL, at least about 21 mL, at least about 22 mL, at least about 23 mL, at least about 24 mL, at least about 25 mL, at least about 26 mL, at least about 27 mL, at least about 28 mL, at least about 29 mL, at least about 30 mL, at least about 31 mL, at least about 32 mL, at least about 33 mL, at least about 34 mL, at least about 35 mL, at least about 36 mL, at least about 37 mL, at least about 38 mL, at least about 39 mL, or at least about 40 mL during the administration period. In some embodiments, the total blood volume in large pulmonary arteries is about 1 mL to about 20 mL during the administration period. In some embodiments, the total blood volume in large pulmonary arteries is about 5 mL to about 15 mL during the administration period. In some embodiments, the total blood volume in large pulmonary arteries is about 5 mL to about 20 mL during the administration period. In some embodiments, the total blood volume in large pulmonary arteries is about 10 mL to about 15 mL during the administration period. In some embodiments, the total blood volume in large pulmonary arteries is about 10.9 mL during the administration period.

In some embodiments, remodeling pulmonary vasculature comprises decreasing the proportion of total blood volume in large pulmonary arteries during the administration period compared to the proportion of total blood volume in large pulmonary arteries prior to the administration period. In some embodiments, decreasing the proportion of total blood volume in large pulmonary arteries comprises decreasing by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 30%, at least about 35%, at least about 40%, at least about 45%, at least about 50%, at least about 55%, at least about 60%, at least about 65%, at least about 70%, or at least about 75% during the administration period compared to the proportion of total blood volume in large pulmonary arteries prior to the administration period. In some embodiments, decreasing the proportion of total blood volume in large pulmonary arteries comprises decreasing by about 10% to about 50% during the administration period compared to the proportion of total blood volume in large pulmonary arteries prior to the administration period. In some embodiments, decreasing the proportion of total blood volume in large pulmonary arteries comprises decreasing by about 5% to about 25% during the administration period compared to the proportion of total blood volume in large pulmonary arteries prior to the administration period. In some embodiments, decreasing the proportion of total blood volume in large pulmonary arteries comprises decreasing by about 5% to about 20% during the administration period compared to the proportion of total blood volume in large pulmonary arteries prior to the administration period. In some embodiments, decreasing the proportion of total blood volume in large pulmonary arteries comprises decreasing by about 5% to about 15% during the administration period compared to the proportion of total blood volume in large pulmonary arteries prior to the administration period. In some embodiments, decreasing the proportion of total blood volume in large pulmonary arteries comprises decreasing by about 10% to about 20% during the administration period compared to the proportion of total blood volume in large pulmonary arteries prior to the administration period. In some embodiments, decreasing the proportion of total blood volume in large pulmonary arteries comprises decreasing by about 10.8% during the administration period compared to the proportion of total blood volume in large pulmonary arteries prior to the administration period.

In some embodiments, the proportion of total blood volume in large pulmonary arteries is at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, or at least about 25% during the administration period. In some embodiments, the proportion of total blood volume in large pulmonary arteries is about 5% to about 25% during the administration period. In some embodiments, the proportion of total blood volume in large pulmonary arteries is about 5% to about 20% during the administration period. In some embodiments, the proportion of total blood volume in large pulmonary arteries is about 5% to about 15% during the administration period. In some embodiments, the proportion of total blood volume in large pulmonary arteries is about 10% to about 20% during the administration period. In some embodiments, the proportion of total blood volume in large pulmonary arteries is about 10% to about 15% during the administration period. In some embodiments, the proportion of total blood volume in large pulmonary arteries is about 12.3% during the administration period.

In some embodiments, remodeling pulmonary vasculature comprises decreasing the proportion of total arterial volume in large pulmonary arteries during the administration period compared to the proportion of total arterial volume in large pulmonary arteries prior to the administration period. In some embodiments, decreasing the proportion of total arterial volume in large pulmonary arteries comprises decreasing by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 30%, at least about 35%, at least about 40%, at least about 45%, at least about 50%, at least about 55%, at least about 60%, at least about 65%, at least about 70%, or at least about 75% during the administration period compared to the proportion of total arterial volume in large pulmonary arteries prior to the administration period.

In some embodiments, decreasing the proportion of total arterial volume in large pulmonary arteries comprises decreasing by about 10% to about 50% during the administration period compared to the proportion of total arterial volume in large pulmonary arteries prior to the administration period. In some embodiments, decreasing the proportion of total arterial volume in large pulmonary arteries comprises decreasing by about 5% to about 25% during the administration period compared to the proportion of total arterial volume in large pulmonary arteries prior to the administration period. In some embodiments, decreasing the proportion of total arterial volume in large pulmonary arteries comprises decreasing by about 5% to about 20% during the administration period compared to the proportion of total arterial volume in large pulmonary arteries prior to the administration period. In some embodiments, decreasing the proportion of total arterial volume in large pulmonary arteries comprises decreasing by about 10% to about 20% during the administration period compared to the proportion of total arterial volume in large pulmonary arteries prior to the administration period. In some embodiments, decreasing the proportion of total arterial volume in large pulmonary arteries comprises decreasing by about 10% to about 15% during the administration period compared to the proportion of total arterial volume in large pulmonary arteries prior to the administration period. In some embodiments, decreasing the proportion of total arterial volume in large pulmonary arteries comprises decreasing by about 12% during the administration period compared to the proportion of total arterial volume in large pulmonary arteries prior to the administration period.

In some embodiments, the proportion of total arterial volume in large pulmonary arteries is at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, or at least about 50% during the administration period. In some embodiments, the proportion of total arterial volume in large pulmonary arteries is about 10% to about 50% during the administration period. In some embodiments, the proportion of total arterial volume in large pulmonary arteries is about 15% to about 45% during the administration period. In some embodiments, the proportion of total arterial volume in large pulmonary arteries is about 15% to about 30% during the administration period. In some embodiments, the proportion of total arterial volume large pulmonary arteries is about 15% to about 25% during the administration period. In some embodiments, the proportion of total arterial volume in large pulmonary arteries is about 20% to about 25% during the administration period. In some embodiments, the proportion of total arterial volume in large pulmonary arteries is about 22.5% during the administration period.

In some embodiments, remodeling pulmonary vasculature comprises increasing the ratio of total blood volume in small pulmonary arteries to total blood volume in large pulmonary arteries during the administration period compared to the ratio of total blood volume in small pulmonary arteries to total blood volume in large pulmonary arteries prior to the administration period. In some embodiments, increasing the ratio comprises increasing by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% during the administration period compared to the ratio prior to the administration period. In some embodiments, increasing the ratio comprises increasing by about 20% to about 60% during the administration period compared to the ratio prior to the administration period. In some embodiments, increasing the ratio comprises increasing by about 20% to about 50% during the administration period compared to the ratio prior to the administration period. In some embodiments, increasing the ratio comprises increasing by about 20% to about 40% during the administration period compared to the ratio prior to the administration period. In some embodiments, increasing the ratio comprises increasing by about 20% to about 35% during the administration period compared to the ratio prior to the administration period. In some embodiments, increasing the ratio comprises increasing by about 30% to about 40% during the administration period compared to the ratio prior to the administration period. In some embodiments, increasing the ratio comprises increasing by about 25% to about 35% during the administration period compared to the ratio prior to the administration period. In some embodiments, increasing the ratio comprises increasing by about 31% during the administration period compared to the ratio prior to the administration period.

In some embodiments, the ratio of total blood volume in small pulmonary arteries to total blood volume in large arteries is at least about 0.5, at least about 0.6, at least about 0.7, at least about 0.8, at least about 0.9, at least about 1.0, at least about 1.1, at least about 1.2, at least about 1.3, at least about 1.4, at least about 1.5, at least about 1.6, at least about 1.7, at least about 1.8, at least about 1.9, at least about 2.0, at least about 2.1, at least about 2.2, at least about 2.3, at least about 2.4, at least about 2.5, at least about 2.6, at least about 2.7, at least about 2.8, at least about 2.9, at least about 3.0, at least about 3.1, at least about 3.2, at least about 3.3, at least about 3.4, at least about 3.5, at least about 3.6, at least about 3.7, at least about 3.8, at least about 3.9, or at least about 4.0 during the administration period. In some embodiments, the ratio of total blood volume in small pulmonary arteries to total blood volume in large arteries is about 2.5 to about 4.0 during the administration period. In some embodiments, the ratio of total blood volume in small pulmonary arteries to total blood volume in large arteries is about 2.0 to about 3.0 during the administration period. In some embodiments, the ratio of total blood volume in small pulmonary arteries to total blood volume in large pulmonary arteries is about 2.2 to about 2.8 during the administration period. In some embodiments, the ratio of total blood volume in small pulmonary arteries to total blood volume in large pulmonary arteries is about 2.4 to about 2.8 during the administration period. In some embodiments, the ratio of total blood volume in small pulmonary arteries to total blood volume in large pulmonary arteries is about 2.6 to about 2.8 during the administration period. In some embodiments, the ratio of total blood volume in small pulmonary arteries to total blood volume in large pulmonary arteries is about 2.7 to about 2.8 during the administration period. In some embodiments, the ratio of total blood volume in small pulmonary arteries to total blood volume in large pulmonary arteries is about 2.7 during the administration period.

In some embodiments, remodeling pulmonary vasculature comprises decreasing the specific image-based fibrosis (SIVFIB) score during the administration period compared to the SIVFIB score prior to the administration period. In some embodiments, decreasing the SIVFIB score comprises decreasing by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, or at least about 20% during the administration period compared to the SIVFIB score prior to the administration period. In some embodiments, decreasing the SIVFIB score comprises decreasing by about 1% to about 10% during the administration period compared to the SIVFIB score prior to the administration period. In some embodiments, decreasing the SIVFIB score comprises decreasing by about 2% to about 10% during the administration period compared to the SIVFIB score prior to the administration period. In some embodiments, decreasing the SIVFIB score comprises decreasing by about 5% to about 10% during the administration period compared to the SIVFIB score prior to the administration period. In some embodiments, decreasing the SIVFIB score comprises decreasing by about 2% to about 6% during the administration period compared to the SIVFIB score prior to the administration period. In some embodiments, decreasing the SIVFIB score comprises decreasing by about 3% to about 5% during the administration period compared to the SIVFIB score prior to the administration period. In some embodiments, decreasing the SIVFIB score comprises decreasing by about 4.6% during the administration period compared to the SIVFIB score prior to the administration period.

In some embodiments, the SIVFIB score is at least about 1.0, at least about 1.5, at least about 2.0, at least about 2.5, at least about 3.0, at least about 3.5, at least about 4.0, at least about 4.5, at least about 5.0, at least about 5.1, at least about 5.2, at least about 5.3, at least about 5.4, at least about 5.5, at least about 5.6, at least about 5.7, at least about 5.8, at least about 5.9, at least about 6.0, at least about 6.1, at least about 6.2, at least about 6.3, at least about 6.4, at least about 6.5, at least about 6.6, at least about 6.7, at least about 6.8, at least about 6.9, at least about 7.0, at least about 7.1, at least about 7.2, at least about 7.3, at least about 7.4, at least about 7.5, at least about 7.6, at least about 7.7, at least about 7.8, at least about 7.9, at least about 8.0, at least about 8.1, at least about 8.2, at least about 8.3, at least about 8.4, at least about 8.5, at least about 8.6, at least about 8.7, at least about 8.8, at least about 8.9, at least about 9.0, at least about 9.1, at least about 9.2, at least about 9.3, at least about 9.4, at least about 9.5, at least about 9.6, at least about 9.7, at least about 9.8, at least about 9.9, at least about 10.0, at least about 10.5, at least about 11.0, at least about 11.5, at least about 12.0, at least about 12.5, at least about 13.0, at least about 13.5, at least about 14.0, at least about 14.5, or at least about 15.0 during the administration period. In some embodiments, the SIVFIB score is about 1 to about 15 during the administration period. In some embodiments, the SIVFIB score is about 5 to about 10 during the administration period. In some embodiments, the SIVFIB score is about 6 to about 10 during the administration period. In some embodiments, the SIVFIB score is about 8 to about 10 during the administration period. In some embodiments, the SIVFIB score is about 8 to about 12 during the administration period. In some embodiments, the SIVFIB score is about 8 to about 9 during the administration period. In some embodiments, the SIVFIB score is about 8.6 during the administration period.

In some embodiments, remodeling pulmonary vasculature further comprises increasing the patient's lung function during the administration period compared to the patient's lung function prior to the administration period.

In some embodiments, remodeling pulmonary vasculature further comprises decreasing the decline in lung function in the patient during the administration period.

In some embodiments, remodeling pulmonary vasculature further comprises improving exercise capacity of the patient during the administration period compared to the exercise capacity of the patient prior to the administration period.

In some embodiments, total blood volume is measured by functional respiratory imaging prior to the administration period and at week 16 of the administration period.

### Pharmaceutical Compositions

In a preferred embodiment of the methods described herein, the compound of the disclosure, e.g., the compound of Formula (I), or enantiomer, diastereomer, or pharmaceutically acceptable salt thereof, is present in a dry powder composition. In one embodiment of a method described herein, the compound of Formula (I), or an enantiomer, diastereomer, or pharmaceutically acceptable salt thereof, is present in a dry powder composition and is administered to a patient via a dry powder inhaler (DPI). In a further embodiment, the dry powder inhaler is a capsule-based dry powder inhaler. The dose of the compound of Formula (I), or enantiomer, diastereomer, or pharmaceutically acceptable salt thereof, is present in a single dry powder capsule or multiple dry powder capsules. Dry powder compositions for use in the methods provided herein are described in international patent application publication nos. WO 2020/223237 and WO 2022/094100, the contents of each of which are incorporated by reference herein in their entireties.

In some embodiments, the dry powder composition comprises(a) a compound of Formula (I), or an enantiomer, diastereomer, or a pharmaceutically acceptable salt thereof, present at from about 0.5 wt % to about 5 wt % of the total weight of the dry powder composition:

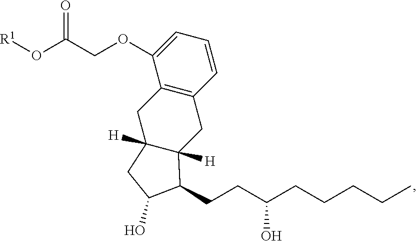

wherein R¹ is tetradecyl, pentadecyl, hexadecyl, heptadecyl, or octadecyl;
(b) from about 10 wt % to about 61 wt % of leucine, and the balance being
(c) a sugar selected from the group consisting of trehalose and mannitol. The entirety of (a), (b), and (c) is 100 wt %.

In a further embodiment, the composition comprises from about 25 wt % to about 61 wt % of leucine. In even a further embodiment, the composition comprises from about 25 wt % to about 45 wt % of leucine. In another embodiment, the composition comprises from about 45 wt % to about 61 wt % of leucine.

In some embodiments, the compound of Formula (I), an enantiomer or diastereomer thereof, or a pharmaceutically acceptable salt thereof is present at about 0.4 wt % about 0.5 wt %, about 1 wt %, about 1.1 wt %, about 1.2 wt %, about 1.3 wt %, about 1.5 wt %, about 1.7 wt %, about 2.0 wt %, about 2.3 wt %, about 2.5 wt %, about 2.6 wt %, about 2.7 wt %, about 2.8 wt %, about 2.9 wt %, about 3 wt %, about 3.1 wt %, about 3.2 wt %, about 3.3 wt %, about 3.4 wt %, about 3.5 wt %, about 4 wt %, about 3.5 wt %, or about 5 wt % of the total weight of the dry powder composition.

The compound of Formula (I) and enantiomers, diastereomers, and pharmaceutically acceptable salts thereof are treprostinil prodrugs as disclosed in International Application Publication WO 2015/061720, the disclosure of which is incorporated herein by reference in its entirety. In some embodiments, the leucine is present at about 25 wt %, about 30 wt %, about 35 wt %, about 40 wt %, about 45 wt %, about 50 wt %, about 55 wt %, or about 60 wt % of the total weight of the dry powder composition.

In one embodiment of the compound of Formula (I), or an enantiomer, diastereomer, or a pharmaceutically acceptable salt thereof, R¹ is tetradecyl. In a further embodiment, R¹ is linear tetradecyl.

In one embodiment of the compound of Formula (I), or an enantiomer, diastereomer, or a pharmaceutically acceptable salt thereof, R¹ is pentadecyl. In a further embodiment, R¹ is linear pentadecyl.

In one embodiment of the compound of Formula (I), or an enantiomer, diastereomer, or a pharmaceutically acceptable salt thereof, R¹ is heptadecyl. In a further embodiment, R¹ is linear heptadecyl.

In one embodiment of the compound of Formula (I), or an enantiomer, diastereomer, or a pharmaceutically acceptable salt thereof, R¹ is octadecyl. In a further embodiment, R¹ is linear octadecyl.

In one embodiment, (a) is a compound of Formula (I) or a pharmaceutically acceptable salt thereof. In a further embodiment, (a) is a compound of Formula (II) or a pharmaceutically acceptable salt thereof. In a further embodiment, (a) is a compound of Formula (II).

In one embodiment, the compound of Formula (I) or (II), or a pharmaceutically acceptable salt thereof, is present at from about 1 wt % to about 5 wt % of the total weight of the dry powder composition. In some embodiments, the compound of Formula (I) or (II), or a pharmaceutically acceptable salt thereof, is present at from about 1 wt % to about 4.5 wt % of the total weight of the dry powder composition. In some embodiments, the compound of Formula (I) or (II) is present at from about 1 wt % to about 4 wt % of the total weight of the dry powder composition.

In one embodiment, the compound of Formula (I) or (II), or a pharmaceutically acceptable salt thereof, is present at from about 1 wt % to about 3.5 wt % of the total weight of the dry powder composition. In another embodiment, the compound of Formula (I) or (II), or a pharmaceutically acceptable salt thereof, is present at from about 1 wt % to about 3 wt % of the total weight of the dry powder composition.

In one embodiment, the compound of Formula (I) or (II), or a pharmaceutically acceptable salt thereof, is present at from about 1 wt % to about 5 wt %, from about 1 wt % to about 4.5 wt %, from about 1 wt % to about 4 wt %, at about 2 wt %, at about 3 wt %, at about 4 wt %, or at about 5 wt %, of the total weight of the dry powder composition. In some embodiments, the compound of Formula (I) or (II), or a pharmaceutically acceptable salt thereof, is present at from about 1 wt % to about 5 wt %, from about 1 wt % to about 4.5 wt %, from about 1 wt % to about 4 wt %, from about 1 wt % to about 2 wt %, about 2 wt %, or about 4 wt %, of the total weight of the dry powder composition.

In one embodiment, the compound of Formula (I) or (II), or a pharmaceutically acceptable salt thereof is present at from about 0.8 wt % to about 3.3 wt %, or from about 1 wt % to about 3 wt %, or from about 1 wt % to about 2 wt %, or from about 1 wt % to about. 1.5% of the total weight of the dry powder composition.

In one embodiment, the compound of Formula (I) or (II), or a pharmaceutically acceptable salt thereof is present at about 1 wt % of the total weight of the dry powder composition. In another embodiment, the compound of Formula (I) or (II), or a pharmaceutically acceptable salt thereof is present at about 1.5 wt % of the total weight of the dry powder composition.

In one embodiment, the compound of Formula (I) or (II), or a pharmaceutically acceptable salt thereof, is present from about 0.8 wt % to about 1.5 wt % of the total weight of the dry powder composition. In another embodiment, the compound of Formula (I) or (II), or a pharmaceutically acceptable salt thereof is present from about 2.7 wt % to about 4 wt % of the total weight of the dry powder composition. In one embodiment, the compound of Formula (I) or (II), or a pharmaceutically acceptable salt thereof is present from about 2.7 wt % to about 3.5 wt %, for example, from about 2.8 wt % to about 3.2 wt %, or from about 2.9 wt % to about 3.1 wt % of the total weight of the dry powder composition.

In one embodiment, the leucine is present at from about 25 wt % to about 61 wt % of the total weight of the dry powder composition. In a further embodiment, the leucine is present at from about 25 wt % to about 50 wt % of the total weight of the dry powder composition. In a further embodiment, the leucine is present at from about 25 wt % to about 40 wt % of the total weight of the dry powder composition. In a further embodiment, the leucine is present at from about 20 wt % to about 33 wt %, e.g., about 20 wt %, about 25 wt %, about 26 wt %, about 27 wt %, about 28 wt %, about 29 wt %, about 30 wt %, about 31 wt %, about 32 wt %, or about 33 wt % of the total weight of the dry powder composition. In a further embodiment, the leucine is present at from about 25 wt % to about 33 wt % of the total weight of the dry powder composition. In a further embodiment, the leucine is present at from about 27 wt % to about 33 wt % of the total weight of the dry powder composition. In a further embodiment, the leucine is present at from about 27 wt % to about 31 wt % of the total weight of the dry powder composition. In a further embodiment, the leucine is present at from about 27 wt % to about 30 wt % of the total weight of the dry powder composition. In a further embodiment, the leucine is present at from about 28 wt % to about 30 wt % of the total weight of the dry powder composition.

In another embodiment, the leucine is present at about 30 wt % of the total weight of the dry powder composition.

In yet another embodiment, the leucine is present at from about 45 wt % to about 61 wt % of the total weight of the dry powder composition, for example at from about 45 wt % to about 55 wt %, or from about 50 wt % to about 55 wt %. In a further embodiment, the compound of Formula (I), or a pharmaceutically acceptable salt thereof, is present at about 3 wt % to about 4 wt % of the total weight of the dry powder composition. In even a further embodiment, R¹ is hexadecyl, e.g., linear hexadecyl.

In some embodiments, the sugar in the dry powder composition is trehalose. In another embodiment, the sugar in the dry powder composition is mannitol.

In one embodiment, the composition has the weight percentages set forth in Table 3, below. In another embodiment, the composition has the weight percentages set forth in Table 3, below, ±5% for each component. In yet another embodiment, the composition has a leucine-to-mannitol weight ratio (“leucine:mannitol” or “leucine-to-mannitol”) set forth in Table 3. In one embodiment, the composition has a weight percentage set forth for composition 10, 11, 12 or 13 in Table 3.

TABLE 3
Exemplary TPIP compositions.

| | Component | | | |
|---|---|---|---|---|
| Composition | TP | Leucine | Mannitol | Leucine-to-Mannitol |
| # | (% w) | (% w) | (% w) | Weight Ratio |
| 1 | 0.5 | 60.0 | 39.5 | 1.52-to-1 |
| 2 | 2.0 | 61.2 | 36.8 | 1.66-to-1 |
| 3 | 3.0 | 60.7 | 36.3 | 1.67-to-1 |
| 4 | 4.0 | 60.0 | 36.0 | 1.67-to-1 |
| 5 | 0.4 | 45.0 | 54.6 | 0.82-to-1 |
| 6 | 1.5 | 44.4 | 54.1 | 0.82-to-1 |
| 7 | 2.0 | 45.0 | 53.0 | 0.85-to-1 |
| 8 | 3.0 | 44.5 | 52.5 | 0.85-to-1 |
| 9 | 4.0 | 45.0 | 51.0 | 0.88-to-1 |
| 10 | 0.5 | 30.0 | 69.5 | 0.43-to-1 |
| 11 | 1.0 | 29.3 | 69.7 | 0.42-to-1 |
| 12 | 1.5 | 29.6 | 68.9 | 0.43-to-1 |
| 13 | 1.5 | 29.3 | 69.2 | 0.42-to-1 |
| 14 | 2.0 | 28.8 | 69.2 | 0.42-to-1 |
| 15 | 3.0 | 28.6 | 68.4 | 0.42-to-1 |
| 16 | 4.0 | 30.0 | 66.0 | 0.45-to-1 |

In one embodiment, the dry powder composition has the components and weight percentages set forth in Table 4.

TABLE 4
Exemplary TPIP composition.

| | Quantity (% w) |
|---|---|
| TP (drug substance) | 1.0 |
| Leucine (dispersion | 29.3 |
| enhancer) | |
| Mannitol (bulking agent) | 69.7 |
| Filled Capsule Weight | 100 |

The leucine-to-sugar (i.e., mannitol or trehalose) weight ratio in a composition provided herein, in one embodiment, is from about 0.4-to-1 (leucine-to-mannitol or -trehalose) to about 1.7-to-1 (leucine-to-mannitol-trehalose). In a further embodiment, the composition comprises a compound of Formula (I), or a pharmaceutically acceptable salt thereof, at from about 1 wt % to about 4 wt % of the total weight of the dry powder composition. In a further embodiment, the leucine-to-sugar weight ratio is from about 0.4:1 (leucine-to-mannitol or -trehalose) to 0.9:1 (leucine-to-mannitol or -trehalose). In even a further embodiment, the leucine-to-sugar weight ratio is from about 0.4:1 (leucine-to-mannitol or -trehalose) to 0.5:1 (leucine-to-mannitol or -trehalose). In a further embodiment, the sugar is mannitol. The leucine, in one embodiment, is L-leucine.

In another embodiment, the sugar is mannitol and the leucine-to-mannitol weight ratio is from about 0.75-to-1 (leucine-to-mannitol) to 0.9-to-1 (leucine-to-mannitol). In a further embodiment, the composition comprises a compound of Formula (I), or a pharmaceutically acceptable salt thereof, at from about 1 wt % to about 4 wt % of the total weight of the dry powder composition. In a further embodiment, the leucine-to-mannitol weight ratio is from about 0.8:1 (leucine-to-mannitol) to 0.9:1 (leucine-to-mannitol). In another embodiment, the sugar is trehalose and the leucine-to-trehalose weight ratio is from about 0.75:1 (leucine-to-trehalose) to 0.9:1 (leucine-to-trehalose). In a further embodiment, the composition comprises a compound of Formula (I), or a pharmaceutically acceptable salt thereof, at from about 1 wt % to about 4 wt % of the total weight of the dry powder composition. In a further embodiment, the leucine-to-trehalose weight ratio is from about 0.8:1 (leucine-to-trehalose) to 0.9:1 (leucine-to-trehalose). The leucine, in one embodiment, is L-leucine.

In yet another embodiment, the sugar is mannitol and the leucine-to-mannitol weight ratio is from about 1.5:1 (leucine-to-mannitol) to 1.7:1 (leucine-to-mannitol). In a further embodiment, the composition comprises a compound of Formula (I), or a pharmaceutically acceptable salt thereof, at from about 1 wt % to about 4 wt % of the total weight of the dry powder composition. In a further embodiment, the leucine-to-mannitol weight ratio is from about 1.6:1 (leucine-to-mannitol) to 1.7:1 (leucine-to-mannitol). In yet another embodiment, the sugar is trehalose and the leucine-to-trehalose weight ratio is from about 1.5:1 (leucine-to-trehalose) to 1.7:1 (leucine-to-trehalose). In a further embodiment, the composition comprises a compound of Formula (I), or a pharmaceutically acceptable salt thereof, at from about 1 wt % to about 4 wt % of the total weight of the dry powder composition. In a further embodiment, the leucine-to-mannitol weight ratio is from about 1.6:1 (leucine-to-trehalose) to 1.7:1 (leucine-to-trehalose).

In another embodiment, the dry powder composition includes (a) about 1-2 wt % of the compound of Formula (I) or (II), an enantiomer or diastereomer thereof, or a pharmaceutically acceptable salt thereof, (b) about 29 wt % of the leucine, and the balance being (c) mannitol. In a further embodiment, (a) in the dry powder composition is about 1 wt % of the compound of Formula (I) or (II), or a pharmaceutically acceptable salt thereof. In another embodiment, (a) in the dry powder composition is at about 2 wt % of the compound of Formula (I) or (II), or a pharmaceutically acceptable salt thereof.

In another embodiment, the dry powder composition includes (a) about 1.5 wt % of the compound of Formula (I) or (II), an enantiomer or diastereomer thereof, or a pharmaceutically acceptable salt thereof, (b) about 29.6 wt % of the leucine, and the balance being (c) mannitol. In a further embodiment, R¹ is linear hexadecyl in the compound of Formula (I).

In another embodiment, the dry powder composition includes (a) about 3 wt % of the compound of Formula (I) or (II), an enantiomer or diastereomer thereof, or a pharmaceutically acceptable salt thereof, (b) about 29 wt % of the leucine, and the balance being (c) mannitol. In a further embodiment, R¹ is linear hexadecyl in the compound of Formula (I).

In another embodiment, the dry powder composition includes (a) about 3 wt % of the compound of Formula (I) or (II), an enantiomer or diastereomer thereof, or a pharmaceutically acceptable salt thereof, (b) about 29 wt % of the leucine, and the balance being (c) mannitol. In a further embodiment, R¹ is linear hexadecyl in the compound of Formula (I).

In another embodiment, the dry powder composition includes (a) about 1 wt % of the compound of Formula (I) or (II), an enantiomer or diastereomer thereof, or a pharmaceutically acceptable salt thereof, (b) about 29 wt % of leucine, and the balance being (c) mannitol. In a further embodiment, R¹ is linear hexadecyl in the compound of Formula (I).

In another embodiment, the dry powder composition includes (a) about 1 wt % of the compound of Formula (I) or (II), an enantiomer or diastereomer thereof, or a pharmaceutically acceptable salt thereof, (b) about 29.6 wt % of the leucine, and the balance being (c) mannitol. In a further embodiment, R¹ is linear hexadecyl in the compound of Formula (I).

In some embodiments, the dry powder composition does not include distearoyl phosphoethanolamine-polyethylene glycol 2000 (DPSE-PEG2000).

In one embodiment, the dry powder composition comprises from about 80 μg to about 1280 μg of a compound of Formula (I) or (II), for example, about 80 μg, about 100 μg, about 110 μg, about 112.5 μg, about 120 μg, about 130 μg, about 140 μg, about 150 μg, about 160 μg, about 170 μg, about 180 μg, about 190 μg, about 200 μg, about 210 μg, about 220 μg, about 225 μg, about 230 μg, about 240 μg, about 250 μg, about 260 μg, about 270 μg, about 280 μg, about 290 μg, about 300 μg, about 310 μg, about 320 μg, about 330 μg, about 340 μg, about 350 μg, about 360 μg, about 370 μg, about 380 μg, about 390 μg, about 400 μg, about 410 μg, about 420 μg, about 430 μg, about 440 μg, about 450 μg, about 460 μg, about 470 μg, about 480 μg, about 490 μg, about 500 μg, about 510 μg, about 520 μg, about 530 μg, about 540 μg, about 550 μg, about 560 μg, about 570 μg, about 580 μg, about 590 μg, about 600 μg, about 610 μg, about 620 μg, about 630 μg, about 640 μg, about 650 μg, about 660 μg, about 670 μg, about 675 μg, about 680 μg, about 690 μg, about 700 μg, about 720 μg, about 740 μg, about 760 μg, about 780 μg, about 800 μg, about 820 μg, about 840 μg, about 860 μg, about 880 μg, about 900 μg, about 920 μg, about 940 pig, about 960 μg, about 980 μg, about 1000 μg, about 1020 μg, about 1040 μg, about 1060 μg, about 1080 μg, about 1100 μg, about 1120 μg, about 1140 μg, about 1160 μg, about 1180 μg, about 1200 μg, about 1220 μg, about 1240 μg, about 1260 μg, or about 1280 μg, of a compound of Formula (I), an enantiomer or diastereomer thereof, or a pharmaceutically acceptable salt thereof, including all values and ranges therein. In one embodiment, the dry powder composition comprises from about 80 μg to about 640 μg of a compound of Formula (I) or (II). In one embodiment, the composition comprises about 80 μg, about 160 μg, about 240 μg, about 320 μg, about 400 μg, about 480 μg, about 640 μg, about 720 μg, about 800 μg, about 960 μg, about 1040 μg, about 1120 μg, about 1200 μg, or about 1280 μg of a compound of Formula (I). In one embodiment, the composition comprises about 80 μg or about 160 μg of a compound of Formula (I). In one embodiment, the composition comprises about 80 μg of a compound of Formula (I). The composition may be present, in one embodiment, in one dry powder capsule or a plurality (two or more) dry powder capsules. When present in multiple capsules, one of the aforementioned doses of the compound of Formula (I) is divided amongst the capsules. The capsule, in one embodiment, is a size #3 HPMC capsule.

Embodiments of a TPIP composition at different unit strengths are provided in Table 5, below. It should be understood that the unit strengths of the components provided herein can be calculated based on the weight percentages of the component and the desired dosage. For example, for an 80 μg dose of TP, each component is multiplied by 80 to obtain the unit strength of each component.

TABLE 5
TPIP filled capsule embodiments.

| | Quantity | Unit Strength (μg/capsule) | | |
|---|---|---|---|---|
| Component | (% w) | 80 | 160 | 320 |
| TP (drug substance) | 1.0 | 80 | 160 | 320 |
| Leucine (dispersion | 29.3 | 2344 | 4688 | 9376 |
| enhancer) | | | | |
| Mannitol (bulking | 69.7 | 5576 | 11152 | 22304 |
| agent) | | | | |
| Filled Capsule | 100 | 8 mg | 16 mg | 32 mg |
| Weight | | | | |

In one embodiment, the dry powder composition comprises about 80 μg of the compound of Formula (I) or a pharmaceutically acceptable salt thereof. In a further embodiment, R¹ is hexadecyl. In even a further embodiment, R¹ is linear hexadecyl.

In one embodiment, the dry powder composition comprises about 160 μg of the compound of Formula (I) or a pharmaceutically acceptable salt thereof. In a further embodiment, R¹ is hexadecyl. In even a further embodiment, R¹ is linear hexadecyl.

In another embodiment, the dry powder composition comprises about 240 μg of the compound of Formula (I) or a pharmaceutically acceptable salt thereof. In a further embodiment, R¹ is hexadecyl. In even a further embodiment, R¹ is linear hexadecyl.

In one embodiment, the dry powder composition comprises about 320 μg of the compound of Formula (I) or a pharmaceutically acceptable salt thereof. In a further embodiment, R¹ is hexadecyl. In even a further embodiment, R¹ is linear hexadecyl.

In another embodiment, the dry powder composition comprises about 400 μg of the compound of Formula (I) or a pharmaceutically acceptable salt thereof. In a further embodiment, R¹ is hexadecyl. In even a further embodiment, R¹ is linear hexadecyl.

In another embodiment, the dry powder composition comprises about 480 μg of the compound of Formula (I) or a pharmaceutically acceptable salt thereof. In a further embodiment, R¹ is hexadecyl. In even a further embodiment, R¹ is linear hexadecyl.

In one embodiment, the dry powder composition comprises about 640 μg of the compound of Formula (I) or a pharmaceutically acceptable salt thereof. In a further embodiment, R¹ is hexadecyl. In even a further embodiment, R¹ is linear hexadecyl.

In one embodiment, the dry powder composition comprises about 720 μg of the compound of Formula (I) or a pharmaceutically acceptable salt thereof. In a further embodiment, R¹ is hexadecyl. In even a further embodiment, R¹ is linear hexadecyl.

In one embodiment, the dry powder composition comprises about 800 μg of the compound of Formula (I) or a pharmaceutically acceptable salt thereof. In a further embodiment, R¹ is hexadecyl. In even a further embodiment, R¹ is linear hexadecyl.

In one embodiment, the dry powder composition comprises about 960 μg of the compound of Formula (I) or a pharmaceutically acceptable salt thereof. In a further embodiment, R¹ is hexadecyl. In even a further embodiment, R¹ is linear hexadecyl.

In one embodiment, the dry powder composition comprises about 1040 μg of the compound of Formula (I) or a pharmaceutically acceptable salt thereof. In a further embodiment, R¹ is hexadecyl. In even a further embodiment, R¹ is linear hexadecyl.

In one embodiment, the dry powder composition comprises about 1120 μg of the compound of Formula (I) or a pharmaceutically acceptable salt thereof. In a further embodiment, R¹ is hexadecyl. In even a further embodiment, R¹ is linear hexadecyl.

In one embodiment, the dry powder composition comprises about 1200 μg of the compound of Formula (I) or a pharmaceutically acceptable salt thereof. In a further embodiment, R¹ is hexadecyl. In even a further embodiment, R¹ is linear hexadecyl.

In one embodiment, the dry powder composition comprises about 1280 μg of the compound of Formula (I) or a pharmaceutically acceptable salt thereof. In a further embodiment, R¹ is hexadecyl. In even a further embodiment, R¹ is linear hexadecyl.

In a preferred embodiment of the dry powder composition provided herein, the leucine is L-leucine.

In another aspect, the present disclosure provides a dry powder composition comprising a compound of Formula (I) or (II), or a pharmaceutically acceptable salt thereof, which provides a particular pharmacokinetic profile following once daily administration. Advantageously, the pharmacokinetic profile of a dry powder composition comprising a compound of Formula (I) or (II), or a pharmaceutically acceptable salt thereof, has a lower Cₘₐₓ and longer half-life compared to the treprostinil inhaled solution, Tyvaso®. In one embodiment, the pharmacokinetic profile of a dry powder composition comprising a compound of Formula (I) or (II), or a pharmaceutically acceptable salt thereof, has a lower Cₘₐₓ and longer half-life compared to the treprostinil inhaled powder, Tyvaso DPI®.

In one embodiment, the dry powder composition exhibiting one of the pharmacokinetic profiles described herein is a composition described in U.S. Patent Application Publication No. 2020/0338005, incorporated by reference herein in its entirety for all purposes.

In another embodiment, the dry powder composition exhibiting one of the pharmacokinetic profiles described herein comprises (a) a compound of Formula (I) or (II) at from about 1 wt % to about 5 wt % of the total weight of the dry powder composition; (b) from about 25 wt % to about 61 wt % of leucine, and the balance being (c) a sugar selected from trehalose and mannitol. The entirety of (a), (b), and (c) is 100 wt %.

### Aerosolized Compositions

The dry powder compositions described herein are in some embodiments, aerosolized via a DPI to provide an aerosolized composition. The aerosolized composition is administered to patient in need of treatment of PH. In another embodiment, the aerosolized composition is administered to patient in need of treatment of pulmonary fibrosis (e.g., PH-ILD where the ILD is pulmonary fibrosis). The aerosolized composition can be characterized by certain parameters known to those of skill in the art, such as mass median aerodynamic diameter (MMAD) and fine particle fraction (FPF).

Mass median aerodynamic diameter (MIMAD) is the value of aerodynamic diameter for which 50% of the mass in a given aerosol is associated with particles smaller than the median aerodynamic diameter (MAD), and 50% of the mass is associated with particles larger than the MAD. MMAD can be determined by impactor measurements, e.g., the Andersen Cascade Impactor (ACT) or the Next Generation Impactor (NGI). In some embodiments, the aerosolized dry powder composition comprises particles with an MMAD of from about 1 μm to about 10 μm, from about 1 μm to about 7 μm, from about 1 μm to about 5 μm, or from about 1 μm to about 4 μm, or from about 1.5 μm to about 3.5 μm, or from about 2 μm to about 3 μm, as measured by NGI. In one embodiment, the dry powder composition exhibiting one of the MMAD profiles provided above comprises mannitol. In another embodiment, the dry powder composition exhibiting the MMAD profile provided above comprises trehalose.

“Fine particle fraction” or “FPF” refers to the fraction of an aerosol having a particle size less than 5 μm in diameter, as measured by cascade impaction. FPF is usually expressed as a percentage. FPF has been demonstrated to correlate to the fraction of the powder that is deposited in the lungs of the subject (e.g., patient). In some embodiments, the dry powder composition is in the form of an aerosol comprising particles with an FPF of at least 20%, at least 30%, at least 40%, at least 50%, from about 30% to about 60%, from about 35% to about 55%, or from about 40% to about 50%, as measured by the NGI. In one embodiment, the aerosolized dry powder composition comprises particles with an FPF of from about 40% to about 70%, from about 30% to about 60%, or from about 50% to about 60%, as measured by NGI. In one embodiment, the dry powder composition exhibiting one of the FPF profiles provided above comprises mannitol. In another embodiment, the dry powder composition exhibiting the FPF profile provided above comprises trehalose.

The dry powder compositions of the present disclosure may be produced from liquid compositions using lyophilization or spray-drying techniques. When lyophilization is used, the lyophilized composition may be milled to obtain the finely divided dry powder containing particles within the desired size range described above. When spray-drying is used, the process is carried out under conditions that result in a finely divided dry powder containing particles within the desired size range described above. Exemplary methods of preparing dry powder forms of pharmaceutical compositions are disclosed in WO 96/32149, WO 97/41833, WO 98/29096, and U.S. Pat. Nos. 5,976,574, 5,985,248, and 6,001,336, the disclosure of each of which is incorporated herein by reference in their entireties. Exemplary spray drying methods are described in U.S. application Publication No. 2020/0338005, and U.S. Pat. Nos. 6,848,197 and 8,197,845, the disclosure of each of which is incorporated herein by reference in their entireties.

In some embodiments, the dry powder compositions of the present disclosure are prepared by the following process. A stock solution of a compound of Formula (I) or (II), an enantiomer or diastereomer thereof, or a pharmaceutically acceptable salt thereof is prepared using an organic solvent, such as an alcohol (e.g., 1-propanol). Aqueous stock solutions of a sugar (e.g., mannitol or trehalose) and leucine are also prepared. Afterwards required amounts of the above stock solutions are added to a mixture of water and the organic solvent to form a spray drying feed solution. In the spray drying feed solution, the volume ratio of water to the organic solvent may be from about 3:2 to about 1:1.

Spray drying is initiated by starting the drying gas flow and heating up the drying gas by setting the desired inlet temperature at, for example, from about 120 °C to about 180 °C, or from about 135 °C to about 150 °C. After the spray dryer outlet temperature reaches a suitable temperature, for example, at from about 55 °C to about 65 °C, the liquid skid inlet is set to allow blank solvents to be atomized with the aid of nitrogen into the spray dryer, and the system is allowed to cool and stabilize. Product filter pulsing is initiated, and product filter purge flow is set, for example, to 10 to 20 standard cubic feet per hour (scfh). After the system stabilizes, the liquid skid inlet is switched to the feed solution prepared above and the process is continued till the feed solution runs out. At the point when the feed solution runs out, the liquid skid inlet is switched back to blank solvents, which are allowed to spray for from about 5 to about 20 minutes. At this point, powder is collected at the bottom of the product filter. After spraying the blank solvent for from about 5 to about 20 minutes, the system is shut down by shutting down the liquid lines, atomization gas, drying gas heater, drying gas inlet and finally the exhaust.

The dry powder compositions of the present disclosure are delivered to the lungs of a subject (e.g., patient) via inhalation using a dry powder inhaler (DPI). In one embodiment, the dry powder inhaler is a single dose dry powder inhaler. A propellant-free device, a DPI delivers dry powder to the lungs of a subject (e.g., patient) using the subject (e.g., patient) inspiration. The unit dose of a dry powder composition used in a DPI device is often a dry powder blister disc of hard capsule. Exemplary DPI devices suitable for delivering the dry powder compositions of the present disclosure include the devices described in the following paragraphs, as well as the DPIs described in U.S. Pat. Nos. 6,766,799, 7,278,425 and 8,496,002, the disclosure of each of which is herein incorporated by reference in their entireties.

The AIR® inhaler (Alkermes) includes a small, breath-activated system that delivers porous powder from a capsule. The porous particles have an aerodynamic diameter of 1-5 μm. See International Patent Application Publication Nos. WO 99/66903 and WO 00/10541, the disclosure of each of which is incorporated herein by reference in their entireties.

Aerolizer™ (Novartis) is a single dose dry powder inhaler. In this device, dry powder medicament is stored in a capsule and released by piercing the capsule wall with TEFLON-coated steel pins. See U.S. Pat. Nos. 6,488,027 and 3,991,761, the disclosure of each of which is incorporated herein by reference in their entireties.

Bang Olufsen provides a breath actuated inhaler using blister strips with up to sixty doses. The dose is made available only during the inhalation by a novel trigger mechanism. The device is equipped with a dose counter and can be disposed of after all doses have been used. See EP 1522325, the disclosure of which is incorporated herein by reference in its entirety.

Clickhaler® (Innovata PLC) is a large reservoir breath-activated multidose device. See U.S. Pat. No. 5,437,270, the disclosure of which is incorporated herein by reference in its entirety.

DirectHaler™ (Direct-Haler A/S) is a single dose, pre-metered, pre-filled, disposable DPI device made from polypropylene. See U.S. Pat. No. 5,797,392, the disclosure of which is incorporated herein by reference in its entirety.

Diskus™ (GIaxoSmithKline) is a disposable small DPI device that holds up to 60 doses contained in double foil blister strips to provide moisture protection. See GB2242134, the disclosure of which is incorporated herein by reference in its entirety.

Eclipse™ (Aventis) is a breath actuated re-usable capsule device capable of delivering up to 20 mg of a dry power composition. The powder is sucked from the capsule into a vortex chamber where a rotating ball assists in powder disaggregation as a subject (e.g., patient) inhales. See U.S. Pat. No. 6,230,707 and WO 9503846, the disclosure of each of which is incorporated herein by reference in their entireties.

Flexhaler® is a plastic breath-activated dry powder inhaler and is amenable for use with the dry powder compositions provided herein.

FlowCaps® (Hovione) is a capsule-based, re-fillable, re-usable passive dry-powder inhaler that holds up to 14 capsules. The inhaler itself is moisture-proof. See U.S. Pat. No. 5,673,686, the disclosure of which is incorporated herein by reference in its entirety.

Gyrohaler® (Vectura) is a passive disposable DPI containing a strip of blisters. See GB2407042, the disclosure of which is incorporated herein by reference in its entirety.

The HandiHaler® (Boehringer Ingelheim GmbH) is a single dose DPI device. It can deliver up to 30 mg of a dry powder composition in capsules. See International Patent Application Publication No. WO 04/024156, the disclosure of which is incorporated herein by reference in its entirety.

MicroDose DPI (Microdose Technologies) is a small electronic DPI device. It uses piezoelectric vibrator (ultrasonic frequencies) to deaggregate the drug powder in an aluminum blister (single or multiple dose). See U.S. Pat. No. 6,026,809, the disclosure of which is incorporated herein by reference in its entirety.

Nektar Dry Powder Inhaler® (Nektar) is a palm-sized and easy-to-use device. It provides convenient dosing from standard capsules and flow-rate-independent lung deposition.

Nektar Pulmonary Inhaler® (Nektar) efficiently removes powders from the packaging, breaks up the particles and creates an aerosol cloud suitable for deep lung delivery. It enables the aerosolized particles to be transported from the device to the deep lung during a subject's (e.g., patient's) breath, reducing losses in the throat and upper airways. Compressed gas is used to aerosolize the powder. See AU4090599 and U.S. Pat. No. 5,740,794, the disclosure of each of which is incorporated herein by reference in their entireties.

NEXT DPI™ is a device featuring multidose capabilities, moisture protection, and dose counting. The device can be used regardless of orientation (upside down) and doses only when proper aspiratory flow is reached. See EP 1196146, U.S. Pat. No. 6,528,096, WO0178693, and WO0053158, the disclosure of each of which is incorporated herein by reference in their entireties.

Neohaler® is a capsule-based plastic breath-activated dry powder inhaler.

Oriel™ DPI is an active DPI that utilizes a piezoelectric membrane and nonlinear vibrations to aerosolize powder formulations. See International Patent Application Publication No. WO 01/68169, the disclosure of which is incorporated herein by reference in its entirety.

The DPI in one embodiment, is a capsule based DPI. In a further embodiment, the capsule based DPI is manufactured by Plastiape. In even a further embodiment, the capsule based DPI is a RS01 monodose dry powder inhaler developed by Plastiape, which features a compact size and a simple and effective perforation system and is suited for both gelatin and HMPC capsules.

Pressair™ is a plastic breath-activated dry powder inhaler.

Pulvinal® inhaler (Chiesi) is a breath-actuated multi-dose (100 doses) dry powder inhaler. The dry powder is stored in a reservoir which is transparent and clearly marked to indicate when the 100th dose has been delivered. See U.S. Pat. No. 5,351,683, the disclosure of which is incorporated herein by reference in its entirety.

The Rotohaler® (GlaxoSmithKline) is a single use device that utilizes capsules. See U.S. Pat. Nos. 5,673,686 and 5,881,721, the disclosure of each of which is incorporated herein by reference in their entireties.

Rexam DPI (Rexam Pharma) is a single dose, reusable device designed for use with capsules. See U.S. Pat. No. 5,651,359 and EP 0707862, the disclosure of each of which is incorporated herein by reference in their entireties.

S2 (Innovata PLC) is a re-useable or disposable single-dose DPI for the delivery of a dry powder composition in high concentrations. Its dispersion mechanism requires minimal effort to achieve excellent drug delivery to the subject's (e.g., patient's) lungs. S2 is easy to use and has a passive engine so no battery or power source is required. See AU3320101, the disclosure of which is incorporated herein by reference in its entirety.

SkyeHaler® DPI (SkyePharma) is a multidose device containing up to 300 individual doses in a single-use, or replaceable cartridge. The device is powered by breath and requires no coordination between breathing and actuation. See U.S. Pat. No. 6,182,655 and WO97/20589, the disclosure of each of which is incorporated herein by reference in their entireties.

Taifun® DPI (LAB International) is a multiple-dose (up to 200) DPI device. It is breath actuated and flow rate independent. The device includes a unique moisture-balancing drug reservoir coupled with a volumetric dose metering system for consistent dosing. See U.S. Pat. No. 6,132,394, the disclosure of which is incorporated herein by reference in its entirety.

The TurboHaler® (AstraZeneca) is described in U.S. Pat. No. 5,983,893, the disclosure of which is incorporated herein by reference in its entirety. This DPI device is an inspiratory flow-driven, multi-dose dry-powder inhaler with a multi-dose reservoir that provides up to 200 doses of a dry powder composition and a dose range from a few micrograms to 0.5 mg.

The Twisthaler® (Schering-Plough) is a multiple dose device with a dose counting feature and is capable of 14-200 actuations. A dry powder composition is packaged in a cartridge that contains a desiccant. See U.S. Pat. No. 5,829,434, the disclosure of which is incorporated herein by reference in its entirety.

Ultrahaler® (Aventis) combines accurate dose metering and good dispersion. It is an easy-to-use, discrete, pocket-sized device with a numerical dose counter, dose taken indicator and a lock-out mechanism. The device is capable of delivering up to 20 mg of a dry powder composition. Ultrahaler® is described in U.S. Pat. No. 5,678,538 and WO2004026380, the disclosure of each of which is incorporated herein by reference in their entireties.

Xcelovair™ (Meridica/Pfizer) holds 60 pre-metered, hermetically sealed doses in the range of 5-20 mg. The device provides moisture protection under accelerated conditions of 40 °C/75% RH. The dispersion system maximizes the fine particle fraction, delivering up to 50% fine particle mass.

In another aspect, a system is provided comprising (i) one of the dry powder compositions described herein and (ii) a dry powder inhaler (DPI) for administration of the dry powder composition. The DPI includes (a) a reservoir comprising the dry powder composition disclosed herein, and (b) a means for introducing the dry powder composition into the subject's lungs via inhalation. The reservoir in one embodiment, comprises the dry powder composition of the present invention in a capsule or in a blister pack. The material for the shell of a capsule can be gelatin, cellulose derivatives, starch, starch derivatives, chitosan, or synthetic plastics. The DPI may be a single dose or a multidose inhaler. In addition, the DPI may be pre-metered or device-metered. In one embodiment, the dry powder inhaler is a single dose dry powder inhaler.

The system, in one embodiment, is used for treating pulmonary hypertension (e.g., group 1 or group 3 PH), portopulmonary hypertension, or pulmonary fibrosis as described in further detail below. The system includes the dry powder composition disclosed herein, i.e., a dry powder composition comprising a compound of Formula (I) or (II), an enantiomer or diastereomer thereof, or a pharmaceutically acceptable salt thereof, and a DPI. In one embodiment, the dry powder composition comprises a compound of Formula (I) or (II), or a pharmaceutically acceptable salt thereof. In another embodiment, the dry powder composition comprises a compound of Formula (I) or (II). The dry powder inhaler may be one described above, may be a single dose or a multidose inhaler, and/or may be pre-metered or device-metered. In one embodiment, the dry powder inhaler is a single dose dry powder inhaler.

In one aspect of the invention, a method for treating pulmonary hypertension (PH) in a patient in need thereof is provided. The method includes administering an effective amount of one of the dry powder compositions disclosed herein to the lungs of the patient via a dry powder inhaler (DPI), once daily during an administration period. The dry powder composition comprises a compound of Formula (I) or (II), or a pharmaceutically acceptable salt thereof. The administering comprises (i) aerosolizing the dry powder composition via a DPI to provide an aerosolized dry powder composition, and (ii) administering the aerosolized dry powder composition to the lungs of the patient via inhalation by the DPI.

### Pharmacokinetic Profile

The pharmacokinetic (PK) profile measured for the compound of Formula (I) or (II), or an enantiomer or diastereomer thereof, or a pharmaceutically acceptable salt thereof, in embodiments described herein, is linear over the dose range of 112.5 μg to 675 μg. Based on this data, the skilled artisan can determine the pharmacokinetic parameters of doses outside of the range, or doses inside this range that were not specifically tested in the Example 1. For example, in order to find a pharmacokinetic parameter at a dose, Cₘₐₓ and AUC associated with specific doses (112.5 μg, 225 μg, 450 μg, and/or 675 μg) may be plotted. The scatter plot may be fit to a straight line, y=mx+b, where in is the slope of the line, b is the y intercept, and the value of an unknown pharmacokinetic parameter (y) may be calculated by plugging in the dose for x. In addition, the dose range of 112.5 μg to 675 μg was based on the molecular weight of the compound of Formula (I) when R¹ is hexadecyl (i.e., the compound of Formula (II)). Equivalent doses for other treprostinil prodrugs (when R¹ is tetradecyl, pentadecyl, heptadecyl, or octadecyl) can be calculated using the molecular weight of the treprostinil prodrug of interest. For example, the dose of the compound of Formula (I) when R¹ is tetradecyl that is equivalent to 112.5 μg of the compound of Formula (II) (R¹ is hexadecyl) can be calculated by multiplying 112.5 μg by the ratio of the molecular weight of the compound of Formula (II) (614.95 μg/mol) to the molecular weight of the compound of Formula (I) when R¹ is tetradecyl (586.9 μg/mol).

In another aspect, the present disclosure provides a dry powder composition comprising a compound of Formula (I) or (II), or a pharmaceutically acceptable salt thereof, which provides a particular pharmacokinetic profile following once daily administration. Advantageously, the pharmacokinetic profile of a dry powder composition comprising a compound of Formula (I) or (II), or a pharmaceutically acceptable salt thereof, has a lower Cₘₐₓ and longer half-life compared to the treprostinil inhaled solution, Tyvaso®. In one embodiment, the pharmacokinetic profile of a dry powder composition comprising a compound of Formula (I) or (II), or a pharmaceutically acceptable salt thereof, has a lower Cₘₐₓ and longer half-life compared to the treprostinil inhaled powder, Tyvaso DPI®.

In embodiments, to the methods of the disclosure, following once daily administration of about 80 μg to about 1280 μg of the compound of Formula (I) or (II), or an enantiomer or diastereomer thereof, or a pharmaceutically acceptable salt thereof, to the lungs of a subject by inhalation, provide at least one of the following characteristics:
(a) a treprostinil maximum plasma concentration (Cₘₐₓ) ranging from about 14 μg/mL to about 3000 μg/mL; or
(b) a treprostinil area under the plasma concentration curve (AUC) ranging from about from about 500 μg*h/mL to about 20000 μg*h/mL.

In a further embodiment, the methods of the disclosure comprising administering about 80 μg, about 112.5 μg, about 160 μg, about 225 μg, about 240 μg, about 320 μg, about 400 μg, about 450 μg, about 480 μg, about 640 μg, about 675 μg, about 720 μg, about 800 μg, about 960 μg, about 1040 μg, about 1120 μg, about 1200 μg, or about 1280 μg of a compound of Formula (I), or an enantiomer or diastereomer thereof, or a pharmaceutically acceptable salt thereof. In a further embodiment, the methods of the disclosure comprise administering about 80 μg, about 160 μg, about 240 μg, about 320 μg, about 400 μg, about 480 μg, about 640 μg, about 720 μg, about 800 Mg, about 960 μg, about 1040 μg, about 1120 μg, about 1200 μg, or about 1280 μg of a compound of Formula (I), or an enantiomer or diastereomer thereof, or a pharmaceutically acceptable salt thereof. In a further embodiment, R¹ is hexadecyl, e.g., linear hexadecyl. The dose administered may be present, in one embodiment, in one dry powder capsule or a plurality (two or more) dry powder capsules. When present in multiple capsules, one of the aforementioned doses of the compound of Formula (I) is divided amongst the capsules.

In some embodiments, following once daily administration of a dry powder composition comprising from about 80 μg to about 1280 μg of the compound of Formula (I), an enantiomer or diastereomer thereof (e.g., a compound of Formula (II)) or a pharmaceutically acceptable salt thereof, the method of use thereof provides a maximum treprostinil plasma concentration (Cₘₐₓ) ranging from about 10 μg/mL to about 3000 pg/mL, for example, about 10 pg/mL, about 15 pg/mL, about 20 pg/mL, about 25 pg/mL about 30 pg/mL, about 35 pg/mL, about 40 pg/mL, about 45 pg/mL, about 50 pg/mL, about 55 pg/mL, about 60 pg/mL, about 65 pg/mL, about 70 pg/mL, about 75 pg/mL, about 80 pg/mL, about 85 pg/mL, about 90 pg/mL, about 95 pg/mL, about 100 pg/mL, about 110 pg/mL, about 120 pg/mL, about 130 pg/mL, about 140 pg/m, about 150 pg/m, about 160 pg/mL, about 170 pg/mL, about 180 pg/mL, about 190 pg/mL, about 200 pg/mL, about 210 pg/mL, about 220 pg/mL, about 230 pg/mL, about 240 pg/mL, about 250 pg/mL, about 260 pg/mL, about 270 pg/mL, about 280 pg/mL, about 290 pg/mL, about 300 pg/mL, about 310 pg/mL, about 320 pg/mL, about 330 pg/mL, about 340 pg/mL, about 350 pg/mL, about 360 pg/mL, about 370 pg/mL, about 380 pg/mL, about 390 pg/mL, about 400 pg/mL, about 410 pg/mL, about 420 pg/mL, about 430 pg/mL, about 440 pg/mL, about 450 pg/mL, about 460 pg/mL, about 470 pg/mL, about 480 pg/mL, about 490 pg/mL, about 500 pg/mL, about 510 pg/mL, about 520 pg/mL, about 530 pg/mL, about 540 pg/mL, about 550 pg/mL, about 560 pg/mL, about 570 pg/mL, about 580 pg/mL, about 590 pg/mL, about 600 pg/mL, about 610 pg/mL, about 620 pg/mL, about 630 pg/mL, about 640 pg/mL, about 650 pg/mL, about 660 pg/mL, about 670 pg/mL, about 680 pg/mL, about 690 pg/mL, about 700 pg/mL, about 750 pg/mL, about 800 pg/mL, about 850 pg/mL, about 900 pg/mL, about 950 pg/mL, about 1000 pg/mL, about 1050 pg/mL, about 1100 pg/mL, about 1150 pg/mL, about 1200 pg/mL, about 1250 pg/mL, about 1300 pg/mL, about 1350 pg/mL, about 1400 pg/mL, about 1450 pg/mL, about 1500 pg/mL, about 1550 pg/mL, about 1600 pg/mL, about 1650 pg/mL, about 1700 pg/mL, about 1750 pg/mL, about 1800 pg/mL, about 1850 pg/mL, about 1900 pg/mL, about 2000 pg/mL, about 2050 pg/mL, about 2100 pg/mL, about 2150 pg/mL, about 2200 pg/mL, about 2250 pg/mL, about 2300 pg/mL, about 2350 pg/mL, about 2400 pg/mL, about 2450 pg/mL, about 2500 pg/mL, about 2550 pg/mL, about 2600 pg/mL, about 2650 pg/mL, about 2700 pg/mL, about 2750 pg/mL, about 2800 pg/mL, about 2850 pg/mL, about 2900 pg/mL, or about 3000 pg/mL including all values and ranges therein.

In some embodiments, following once daily administration of about 80 μg to about 1280 μg of the compound of Formula (I), or an enantiomer or diastereomer thereof (e.g., a compound of Formula (II)) or a pharmaceutically acceptable salt thereof, the dry power composition or method of the disclosure provides an area under the plasma concentration curve (AUC) ranging from about 300 pg*hr/mL to about 20000 pg*hr/mL, for example, about 300 pg*hr/mL, about 400 pg*hr/mL, about 500 pg*hr/mL, about 600 pg*hr/mL, about 700 pg*hr/mL, about 800 pg*hr/mL, about 900 pg*hr/mL, about 1000 pg*hr/mL, about 1100 pg*hr/mL, about 1200 pg*hr/mL, about 1300 pg*hr/mL, about 1400 pg*hr/mL, about 1500 pg*hr/mL, about 1600 pg*hr/mL, about 1700 pg*hr/mL, about 1800 pg*hr/mL, about 1900 pg*hr/mL, about 2000 pg*hr/mL, about 2100 pg*hr/mL, about 2200 pg*hr/mL, about 2300 pg*hr/mL, about 2400 pg*hr/mL, about 2500 pg*hr/mL, about 2600 pg*hr/mL, about 2700 ng*h/mL, about 2800 ng*h/mL, about 2900 pg*hr/mL, about 3000 pg*hr/mL, about 3100 pg*hr/mL, about 3200 pg*hr/mL, about 3300 pg*hr/mL, about 3400 pg*hr/mL, about 3500 pg*hr/mL, about 3600 pg*h/mL, about 3700 pg*hr/mL, about 3800 pg*hr/mL, about 3900 pg*hr/mL, about 4000 pg*hr/mL, about 4100 pg*hr/mL, about 4200 pg*hr/mL, about 4300 pg*hr/mL, about 4400 pg*hr/mL, about 4500 pg*hr/mL, about 4600 pg*hr/mL, about 4675 pg*hr/mL, about 4700 pg*hr/mL, about 4800 pg*hr/mL, about 4900 pg*hr/mL, about 5000 pg*hr/mL, about 5100 pg*hr/mL, about 5200 pg*hr/mL, about 5300 pg*hr/mL, about 5400 pg*hr/mL, about 5500 pg*hr/mL, about 5600 pg*hr/mL, about 5700 pg*hr/mL, about 5800 pg*hr/mL, about 5900 pg*hr/mL, about 6000 pg*hr/mL, about 6100 pg*hr/mL, about 6200 pg*hr/mL, about 6300 pg*hr/mL*hr/mL, about 6400 pg*hr/mL, about 6500 pg*hr/mL*hr/mL, about 6600 pg*hr/mL, about 6700 pg*hr/mL, about 6800 pg*hr/mL, about 6900 pg*hr/mL, about 7000 pg*hr/mL, about 7100 pg*hr/mL, about 7200 pg*hr/mL, about 7300 pg*hr/mL, about 7400 pg*hr/mL, about 7500 pg*hr/mL, about 7510 pg*hr/mL, about 7600 pg*hr/mL, about 7700 pg*hr/mL, about 7800 pg*hr/mL, about 7900 pg*hr/mL, about 8000 pg*hr/mL, about 8100 pg*hr/mL, about 8200 pg*hr/mL, about 8300 pg*hr/mL, about 8400 pg*hr/mL, about 8500 pg*hr/mL, about 8600 pg*hr/mL, about 8700 pg*hr/mL, about 8800 pg*hr/mL, about 8900 pg*hr/mL, about 9000 pg*hr/mL, about 9100 pg*hr/mL, about 9200 pg*hr/mL, about 9300 pg*hr/mL, about 9400 pg*hr/mL, about 9500 pg*hr/mL, about 9600 pg*hr/mL, about 9700 pg*hr/mL, about 9800 pg*hr/mL, about 9900 pg*hr/mL, about 10000 pg*hr/mL, about 10100 pg*hr/mL, about 10200 pg*hr/mL, about 10300 pg*hr/mL, about 10400 pg*hr/mL, about 10500 pg*hr/mL, about 10600 pg*hr/mL, about 10700 pg*hr/mL, about 10800 pg*hr/mL, about 10900 pg*hr/mL, about 11000 pg*hr/mL, about 11100 pg*hr/mL, about 11200 pg*hr/mL, about 11300 pg*hr/mL, about 11400 pg*hr/mL, about 11500 pg*hr/mL, about 11600 pg*hr/mL, about 11700 pg*hr/mL, about 11800 pg*hr/mL, about 11900 pg*hr/mL about 12000 pg*hr/mL, about 12100 pg*hr/mL about 12200 pg*hr/mL, about 12300 pg*hr/mL, about 12400 pg*hr/mL, about 12500 pg*hr/mL, about 12600 pg*hr/mL, about 12700 pg*hr/mL, about 12800 pg*hr/mL, about 12900 pg*hr/mL, about 13000 pg*hr/mL, about 13100 pg*hr/mL, about 13200 pg*hr/mL, about 13300 pg*hr/mL, about 13400 pg*hr/mL, about 13500 pg*hr/mL, about 13600 pg*hr/mL, about 13700 pg*hr/mL, about 13800 pg*hr/mL, about 13900 pg*hr/mL, about 14000 pg*hr/mL, about 14100 pg*hr/mL, about 14200 pg*hr/mL, about 14300 pg*hr/mL, about 14400 pg*hr/mL, about 14500 pg*hr/mL, about 14600 pg*hr/mL, about 14700 pg*hr/mL, about 14800 pg*hr/mL, about 14900 pg*hr/mL, about 15000 pg*hr/mL, about 15100 pg*hr/mL, about 15200 pg*hr/mL, about 15300 pg*hr/mL, about 15400 pg*hr/mL, about 15500 pg*hr/mL, about 15600 pg*hr/mL, about 15700 pg*hr/mL, about 15800 pg*hr/mL, about 15900 pg*hr/mL, about 16000 pg*hr/mL, about 16100 pg*hr/mL, about 16200 pg*hr/mL, about 16300 pg*hr/mL, about 16400 pg*hr/mL, about 16500 pg*hr/mL, about 16600 pg*hr/mL, about 16700 pg*hr/mL, about 16800 pg*hr/mL, about 16900 pg*hr/mL, about 17000 pg*hr/mL, about 17100 pg*hr/mL, about 17200 pg*hr/mL, about 17300 pg*hr/mL, about 17400 pg*hr/mL, about 17500 pg*hr/mL, about 17600 pg*hr/mL, about 17700 pg*hr/mL, about 17800 pg*hr/mL, about 17900 pg*hr/mL, about 18000 pg*hr/mL, about 18100 pg*hr/mL, about 18200 pg*hr/mL, about 18300 pg*hr/mL, about 18400 pg*hr/mL, about 18500 pg*hr/mL, about 18600 pg*hr/mL, about 18700 pg*hr/mL, about 18800 pg*hr/mL, about 18900 pg*hr/mL, about 19000 pg*hr/mL, about 19100 pg*hr/mL, about 19200 pg*hr/mL, about 19300 pg*hr/mL, about 19400 pg*hr/mL, about 19500 pg*hr/mL, about 19600 pg*hr/mL, about 19700 pg*hr/mL, about 19800 pg*hr/mL, about 19900 pg*hr/mL, or about 20000 pg*hr/mL, including all values and ranges therein. In some embodiments, following once daily administration of about 80 μg to about 1280 μg of the compound of Formula (I), or an enantiomer or diastereomer thereof (e.g., a compound of Formula (II)) or a pharmaceutically acceptable salt thereof, the dry power composition or method of the disclosure provides an area under the plasma concentration curve (AUC) of about 4675 pg*hr/mL. In some embodiments, following once daily administration of about 80 g to about 1280 μg of the compound of Formula (I), or an enantiomer or diastereomer thereof (e.g., a compound of Formula (II)) or a pharmaceutically acceptable salt thereof, the dry power composition or method of the disclosure provides an area under the plasma concentration curve (AUC) of about 7510 pg*hr/mL.

In some embodiments, the method of disclosure achieves treprostinil plasma trough concentration during an administration period of the dry powder composition. In some embodiments, the plasma trough levels are sufficient to provide a sustained therapeutic response during the administration period. In some embodiments, following once daily administration of about 80 μg to about 1280 μg of the compound of Formula (I) or an enantiomer or diastereomer thereof (e.g., where R¹ is hexadecyl, e.g., linear hexadecyl), the subject (e.g., patient) has a treprostinil plasma trough concentration of at least about 1 pg/mL, about 2 pg/mL, about 3 pg/mL, about 4 pg/mL, about 5 pg/mL, about 10 pg/mL, about 15 pg/mL, about 20 pg/mL, about 25 pg/mL about 30 pg/mL, about 35 pg/mL, about 40 pg/mL, about 45 pg/mL, about 50 pg/mL, about 55 pg/mL, about 60 pg/mL, about 65 pg/mL, about 70 pg/mL, about 75 pg/mL, about 80 pg/mL, about 85 pg/mL, about 90 pg/mL, about 95 pg/mL, about 100 pg/mL, about 110 pg/mL, about 120 pg/mL, about 130 pg/mL, about 140 pg/mL, about 150 pg/mL, about 160 pg/mL, about 170 pg/mL, about 180 pg/mL, about 190 pg/mL, about 200 pg/mL, about 210 pg/mL, about 220 pg/mL, about 230 pg/mL, about 240 pg/mL, about 250 pg/mL, about 260 pg/mL, about 270 pg/mL, about 280 pg/mL, about 290 pg/mL, about 300 pg/mL, about 310 pg/mL, about 320 pg/mL, about 330 pg/mL, about 340 pg/mL, about 350 pg/mL, about 360 pg/mL, about 370 pg/mL, about 380 pg/mL, about 390 pg/mL, or about 400 pg/mL, including all values and ranges therein.

In some embodiments, following once daily administration of about 80 μg to about 1280 μg of the compound of Formula (I) or an enantiomer, diastereomer thereof (e.g., a compound of Formula (II), or pharmaceutically acceptable salt thereof, the subject (e.g., patient) has at least one of the following characteristics:
(a) a maximum treprostinil plasma concentration (Cₘₐₓ) within about 80% to about 125% of the range of from about 17 pg/mL to about 2300 pg/mL, for example, about 13 pg/mL, about 14 pg/mL, about 15 pg/mL, about 20 pg/mL, about 25 pg/mL, about 30 pg/mL, about 35 pg/mL, about 40 pg/mL, about 45 pg/mL, about 50 pg/mL, about 55 pg/mL, about 60 pg/mL, about 65 pg/mL, about 70 pg/mL, about 75 pg/mL, about 80 pg/mL, about 85 pg/mL, about 90 pg/mL, about 95 pg/mL, about 100 pg/mL, about 110 pg/mL, about 120 pg/mL, about 130 pg/mL, about 140 pg/mL, about 150 pg/mL, about 160 pg/mL, about 170 pg/mL, about 180 pg/mL, about 190 pg/mL, about 200 pg/mL, about 210 pg/mL, about 220 pg/mL, about 230 pg/mL, about 240 pg/mL, about 250 pg/mL, about 260 pg/mL, about 270 pg/mL, about 280 pg/mL, about 290 pg/mL, about 300 pg/mL, about 310 pg/mL, about 320 pg/mL, about 330 pg/mL, about 340 pg/mL, about 350 pg/mL, about 360 pg/mL, about 370 pg/mL, about 380 pg/mL, about 390 pg/mL, about 400 pg/mL, about 410 pg/mL, about 420 pg/mL, about 430 pg/mL, about 440 pg/mL, about 450 pg/mL, about 460 pg/mL, about 470 pg/mL, about 480 pg/mL, about 490 pg/mL, about 500 pg/mL, about 510 pg/mL, about 520 pg/mL, about 530 pg/mL, about 540 pg/mL, about 550 pg/mL, about 560 pg/mL, about 570 pg/mL, about 580 pg/mL, about 590 pg/mL, about 600 pg/mL, about 610 pg/mL, about 620 pg/mL, about 630 pg/mL, about 640 pg/mL, about 650 pg/mL, about 660 pg/mL, about 670 pg/mL, about 680 pg/mL, about 690 pg/mL, about 700 pg/mL, about 750 pg/mL, about 800 pg/mL, about 850 pg/mL, about 900 pg/mL, about 950 pg/mL, about 1000 pg/mL, about 1050 pg/mL, about 1100 pg/mL, about 1150 pg/mL, about 1200 pg/mL, about 1250 pg/mL, about 1300 pg/mL, about 1350 pg/mL, about 1400 pg/mL, about 1450 pg/mL, about 1500 pg/mL, about 1550 pg/mL, about 1600 pg/mL, about 1650 pg/mL, about 1700 pg/mL, about 1750 pg/mL, about 1800 pg/mL, about 1850 pg/mL, about 1900 pg/mL, about 2000 pg/mL, about 2050 pg/mL, about 2100 pg/mL, about 2150 pg/mL, about 2200 pg/mL, about 2250 pg/mL, about 2300 pg/mL, about 2350 pg/mL, about 2400 pg/mL, about 2450 pg/mL, about 2500 pg/mL, about 2550 pg/mL, about 2600 pg/mL, about 2650 pg/mL, about 2700 pg/mL, about 2750 pg/mL, about 2800 pg/mL, about 2850 pg/mL, about 2900 pg/mL, or about 3000 pg/mL, including all values and ranges therein; or
(b) a treprostinil area under the plasma concentration curve (AUC_{0-inf}) within about 80% to about 125% of the range of from about 475 pg*h/mL to about 15500 pg*h/mL, for example, about 370 pg*h/mL, about 400 pg*h/mL, about 450 pg*h/ml, about 500 pg*b/mL, about 550 pg*h/mL, about 600 pg*h/mL, about 650 pg*h/mL, about 700 pg*h/mL, about 800 pg*h/mL, about 900 pg*h/mL, about 1000 pg*h/mL, about 1100 pg*h/mL, about 1200 pg*h/mL, about 1300 pg*h/mL, about 1400 pg*h/mL, about 1500 pg*h/mL, about 1600 pg*h/mL, about 1700 pg*h/mL, about 1800 pg*h/mL, about 1900 pg*h/mL, about 2000 pg*h/mL, about 2100 pg*h/mL, about 2200 pg*h/ml, about 2300 pg*h/mL, about 2400 pg*b/mL, about 2500 pg*h/mL, about 2600 pg*h/mL, about 2700 ng*h/mL, about 2800 ng*h/mL, about 2900 pg*h/mL, about 3000 pg*h/mL, about 3100 pg*h/mL, about 3200 pg*h/mL, about 3300 pg*h/mL, about 3400 pg*h/mL, about 3500 pg*h/mL, about 3600 pg*h/mL, about 3700 pg*h/mL, about 3800 pg*h/mL, about 3900 pg*h/m L, about 4000 pg*h/mL, about 4100 pg*h/mL, about 4200 pg*h/mL, about 4300 pg*h/mL, about 4400 pg*h/mL, about 4500 pg*h/mL, about 4600 pg*h/mL, about 4700 pg*h/mL, about 4800 pg*h/mL, about 4900 pg*h/mL, about 5000 pg*h/mL, about 5100 pg*hr/mL, about 5200 pg*hr/mL, about 5300 pg*hr/mL, about 5400 pg*hr/mL, about 5500 pg*hr/mL, about 5600 pg*hr/mL, about 5700 pg*hr/mL, about 5800 pg*hr/mL, about 5900 pg*hr/mL, about 6000 pg*hr/mL, about 6100 pg*hr/mL, about 6200 pg*hr/mL, about 6300 pg*h/mL, about 6400 pg*h/mL, about 6500 pg*h/mL, about 6600 pg*hr/mL, about 6700 pg*hr/mL, about 6800 pg*hr/mL, about 6900 pg*hr/mL, about 7000 pg*hr/mL, about 7100 pg*hr/mL, about 7200 pg*hr/mL, about 7300 pg*hr/mL, about 7400 pg*hr/mL, about 7500 pg*hr/mL, about 7600 pg*hr/mL, about 7700 pg*hr/mL, about 7800 pg*hr/mL, about 7900 pg*hr/mL, about 8000 pg*hr/mL, about 8100 pg*hr/mL, about 8200 pg*hr/mL, about 8300 pg*h/mL, about 8400 pg*h/mL, about 8500 pg*h/mL, about 8600 pg*hr/mL, about 8700 pg*hr/mL, about 8800 pg*hr/mL, about 8900 pg*hr/mL, about 9000 pg*hr/mL, about 9100 pg*hr/mL, about 9200 pg*hr/mL, about 9300 pg*hr/mL, about 9400 pg*hr/mL, about 9500 pg*hr/mL, about 9600 pg*hr/mL, about 9700 pg*hr/mL, about 9800 pg*hr/mL, about 9900 pg*hr/mL, about 10000 pg*hr/mL, about 10100 pg*hr/mL, about 10200 pg*hr/mL, about 10300 pg*hr/mL, about 10400 pg*hr/mL, about 10500 pg*hr/mL, about 10600 pg*hr/mL, about 10700 pg*hr/mL, about 10800 pg*hr/mL, about 10900 pg*hr/mL, about 11000 pg*hr/mL, about 11100 pg*hr/mL, about 11200 pg*hr/mL, about 11300 pg*hr/mL, about 11400 pg*hr/mL, about 11500 pg*hr/mL, about 11600 pg*hr/mL, about 11700 pg*hr/mL, about 11800 pg*hr/mL, about 11900 pg*hr/mL, about 12000 pg*hr/mL, about 12100 pg*hr/mL, about 12200 pg*hr/mL, about 12300 pg*hr/mL, about 12400 pg*hr/mL, about 12500 pg*hr/mL, about 12600 pg*hr/mL, about 12700 pg*hr/mL, about 12800 pg*hr/mL, about 12900 pg*hr/mL, about 13000 pg*hr/mL, about 13100 pg*lur/mL, about 13200 pg*hr/mL, about 13300 pg*hr/mL, about 13400 pg*hr/mL, about 13500 pg*hr/mL, about 13600 pg*hr/mL, about 13700 pg*hr/mL, about 13800 pg*hr/mL, about 13900 pg*hr/mL, about 14000 pg*hr/mL, about 14100 pg*hr/mL, about 14200 pg*hr/mL, about 14300 pg*hr/mL, about 14400 pg*hr/mL, about 14500 pg*hr/mL, about 14600 pg*hr/mL, about 14700 pg*hr/mL, about 14800 pg*hr/mL, about 14900 pg*hr/mL, about 15000 pg*hr/mL, about 15100 pg*hr/mL, about 15200 pg*hr/mL, about 15300 pg*hr/mL, about 15400 pg*hr/mL, about 15500 pg*hr/mL, about 15600 pg*hr/mL, about 15700 pg*hr/mL, about 15800 pg*hr/mL, about 15900 pg*hr/mL, about 16000 pg*hr/mL, about 16100 pg*hr/mL, about 16200 pg*hr/mL, about 16300 pg*hr/mL, about 16400 pg*hr/mL, about 16500 pg*hr/mL, about 16600 pg*hr/mL, about 16700 pg*hr/mL, about 16800 pg*hr/mL, about 16900 pg*hr/mL, about 17000 pg*hr/mL, about 17100 pg*hr/mL, about 17200 pg*hr/mL, about 17300 pg*hr/mL, about 17400 pg*hr/mL, about 17500 pg*hr/mL, about 17600 pg*hr/mL, about 17700 pg*hr/mL, about 17800 pg*hr/mL, about 17900 pg*hr/mL, about 18000 pg*hr/mL, about 18100 pg*hr/mL, about 18200 pg*hr/mL, about 18300 pg*hr/mL, about 18400 pg*hr/mL, about 18500 pg*hr/mL, about 18600 pg*hr/mL, about 18700 pg*hr/mL, about 18800 pg*hr/mL, about 18900 pg*hr/mL, about 19000 pg*hr/mL, about 19100 pg*hr/mL, about 19200 pg*hr/mL, about 19300 pg*hr/mL, about 19400 pg*hr/mL, about 19500 pg*hr/mL, about 19600 pg*hr/mL, about 19700 pg*hr/mL, about 19800 pg*hr/mL, about 19900 pg*hr/mL, or about 20000 pg*hr/mL, including all values and ranges therein.

In some embodiments, about 80 μg of the compound of Formula (II) or enantiomer or diastereomer thereof, or pharmaceutically acceptable salt thereof, is administered once daily, and provides a treprostinil Cₘₐₓ ranging from about 14 μg/mL to about 155 μg/mL, for example, about 14 pg/mL, about 15 μg/mL, about 20 μg/mL, about 25 μg/mL about 30 μg/mL, about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, about 85 μg/mL, about 90 μg/mL, about 95 μg/mL, about 100 μg/mL, about 105 μg/mL about 110 μg/mL, about 115 μg/mL, about 120 μg/mL, about 125 μg/mL, about 130 μg/mL, about 135 μg/mL, about 140 μg/mL, about 145 μg/mL, about 150 μg/mL, and about 155 μg/mL, including all values and ranges therein. In some embodiments, about 80 μg of the compound of Formula (II) or enantiomer or diastereomer thereof, or pharmaceutically acceptable salt thereof, is administered once daily and provides a treprostinil Cₘₐₓ of about 80%-125% of a range from about 17 pg/mL to about 125 pg/mL. In some embodiments, about 80 μg of the compound of Formula (II), or enantiomer or diastereomer thereof, or pharmaceutically acceptable salt thereof, is administered once daily and provides a treprostinil Cₘₐₓ of about 80%-125% of a range from about 35 pg/mL to about 105 pg/mL.

In some embodiments, about 112.5 μg of the compound of Formula (II) or enantiomer or diastereomer thereof, or pharmaceutically acceptable salt thereof, is administered and provides a treprostinil Cₘₐₓ (CV %) ranging from about 80% to about 125% of about 78.4 (72.9) pg/mL.

In some embodiments, about 160 μg of the compound of Formula (II), or enantiomer or diastereomer thereof, or pharmaceutically acceptable salt thereof, is administered once daily, and provides a treprostinil Cₘₐₓ ranging from about 30 pg/mL to about 335 pg/mL, for example, about 30 pg/mL, about 35 pg/mL, about 40 pg/mL, about 45 pg/mL, about 50 pg/mL, about 55 pg/mL, about 60 pg/mL, about 65 pg/mL, about 70 pg/mL, about 75 pg/mL, about 80 pg/mL, about 85 pg/mL, about 90 pg/mL, about 95 pg/mL, about 100 pg/mL, about 105 pg/mL about 110 pg/mL, about 115 pg/mL, about 120 pg/mL, about 125 pg/mL, about 130 pg/mL, about 135 pg/mL, about 140 pg/mL, about 145 pg/mL, about 150 pg/mL, about 155 pg/mL, about 160 pg/mL, about 165 pg/mL, about 170 pg/mL, about 175 pg/mL, about 180 pg/mL, about 1850 pg/mL, about 190 pg/mL, about 195 pg/mL, about 200 pg/mL, about 205 pg/mL, about 210 pg/mL, about 215 pg/mL, about 220 pg/mL, about 225 pg/mL, about 230 pg/mL, about 235 pg/mL, about 240 pg/mL, about 245 pg/mL, about 250 pg/mL, about 255 pg/mL, about 260 pg/mL, about 265 pg/mL, about 270 pg/mL, about 275 pg/mL, about 280 pg/mL, about 285 pg/mL, about 290 pg/mL, about 295 pg/mL, about 300 pg/mL, about 305 pg/mL, about 310 pg/mL, about 315 pg/mL, about 320 pg/mL, about 325 pg/mL, about 330 pg/mL, about 335 pg/mL, about 340 pg/mL, about 345 pg/mL, or about 350 pg/mL, including all values and ranges therein. In some embodiments, about 160 μg of the compound of Formula (II), or enantiomer or diastereomer thereof, or pharmaceutically acceptable salt thereof, is administered once daily and provides a treprostinil Cₘₐₓ from about 80%-125% of a range from about 35 pg/mL to about 270 pg/mL. In some embodiments, about 160 μg of the compound of Formula (II), or enantiomer or diastereomer thereof, or pharmaceutically acceptable salt thereof, is administered once daily and provides a treprostinil Cₘₐₓ from about 80%-125% of a range from about 76 pg/mL to about 230 pg/mL.

In some embodiments, about 225 μg of the compound of Formula (II), or enantiomer or diastereomer thereof, or pharmaceutically acceptable salt thereof, is administered once daily, and provides a treprostinil Cₘₐₓ ranging from about 80% to about 125% of about 287 (46.6) pg/mL. In some embodiments, the dry powder composition comprises about 225 μg of the compound of Formula (II) or enantiomer or diastereomer thereof, or pharmaceutically acceptable salt thereof, and provides a steady state treprostinil Cₘₐₓ ranging from about 80% to about 125% of about 193 (32.9) pg/mL. In some embodiments, the about 225 μg of the compound of Formula (II), or enantiomer or diastereomer thereof, or pharmaceutically acceptable salt thereof, is administered once daily, and provides a steady state treprostinil Cₘₐₓ (CV %) ranging from about 80% to about 125% of about 228 (46.4) pg/mL.

In some embodiments, about 240 μg of the compound of Formula (II), or enantiomer or diastereomer thereof, or pharmaceutically acceptable salt thereof, is administered once daily, and provides a treprostinil Cₘₐₓ ranging from about 45 pg/mL to about 520 pg/mL, for example, about 45 pg/mL, about 50 pg/mL, about 60 pg/mL, about 70 pg/mL, about 80 pg/mL, about 90 pg/mL, about 100 pg/mL, about 110 pg/mL, about 120 pg/mL, about 130 pg/mL, about 140 pg/mL, about 150 pg/mL, about 160 pg/mL, about 170 pg/mL, about 180 pg/mL, about 190 pg/mL, about 200 pg/mL, about 210 pg/mL, about 220 pg/mL, about 230 pg/mL, about 240 pg/mL, about 250 pg/mL, about 260 pg/mL, about 270 pg/mL, about 280 pg/mL, about 290 pg/mL, about 300 pg/mL, about 310 pg/mL, about 320 pg/mL, about 330 pg/mL, about 340 pg/mL, about 350 pg/mL, about 360 pg/mL, about 370 pg/mL, about 380 pg/mL, about 390 pg/mL, about 400 pg/mL, about 410 pg/mL, about 420 pg/mL, about 430 pg/mL, about 440 pg/mL, about 450 pg/mL, about 460 pg/mL, about 470 pg/mL, about 480 pg/mL, about 490 pg/mL, about 500 pg/mL, about 510 pg/mL, or about 520 pg/mL, including all values and ranges therein. In some embodiments, about 240 pg of the compound of Formula (II), or enantiomer or diastereomer thereof, or pharmaceutically acceptable salt thereof, is administered once daily and provides a treprostinil Cₘₐₓ from about 80%-125% of a range from about 55 pg/mL to about 415 pg/mL. In some embodiments, about 240 μg of the compound of Formula (II), or enantiomer or diastereomer thereof, or pharmaceutically acceptable salt thereof, is administered once daily and provides a treprostinil Cₘₐₓ from about 80%-125% of a range from about 115 pg/mL to about 355 pg/mL.

In some embodiments, about 320 μg of the compound of Formula (II), or enantiomer or diastereomer thereof, or pharmaceutically acceptable salt thereof, is administered once daily, and provides a treprostinil Cₘₐₓ ranging from about 60 pg/mL to about 700 pg/mL, for example, about 60 pg/mL, about 70 pg/mL, about 80 pg/mL, about 90 pg/mL, about 100 pg/mL, about 110 pg/mL, about 120 pg/mL, about 130 pg/mL, about 135 pg/mL, about 140 pg/mL, about 140 pg/mL, about 150 pg/mL, about 160 pg/mL, about 170 pg/mL, about 180 pg/mL, about 190 pg/mL, about 200 pg/mL, about 210 pg/mL, about 220 pg/mL, about 230 pg/mL, about 240 pg/mL, about 250 pg/mL, about 260 pg/mL, about 270 pg/mL, about 280 pg/mL, about 290 pg/mL, about 300 pg/mL, about 310 pg/mL, about 320 pg/mL, about 330 pg/mL, about 340 pg/mL, about 350 pg/mL, about 360 pg/mL, about 370 pg/mL, about 380 pg/mL, about 390 pg/mL, about 400 pg/mL, about 410 pg/mL, about 420 pg/mL, about 430 pg/mL, about 440 pg/mL, about 450 pg/mL, about 460 pg/mL, about 470 pg/mL, about 480 pg/mL, about 490 pg/mL, about 500 pg/mL, about 510 pg/mL, about 520 pg/mL, about 530 pg/mL, about 540 pg/mL, about 550 pg/mL, about 560 pg/mL, about 570 pg/mL, about 580 pg/mL, about 590 pg/mL, about 600 pg/mL, about 610 pg/mL, about 620 pg/mL, about 630 pg/mL, about 640 pg/mL, about 650 pg/mL, about 660 pg/mL, about 670 pg/mL, about 680 pg/mL, about 690 pg/mL, or about 700 pg/mL, including all values and ranges therein. In some embodiments, about 320 μg of the compound of Formula (II), or enantiomer or diastereomer thereof, or pharmaceutically acceptable salt thereof, is administered once daily and provides a treprostinil Cₘₐₓ from about 80%-125% of a range from about 80 μg/mL to about 560 μg/mL. In some embodiments, about 320 μg of the compound of Formula (II), or enantiomer or diastereomer thereof, or pharmaceutically acceptable salt thereof, is administered once daily and provides a treprostinil Cₘₐₓ from about 80%-125% of a range from about 160 μg/mL to about 480 μg/mL.

In some embodiments, about 400 μg of the compound of Formula (II), or enantiomer or diastereomer thereof, or pharmaceutically acceptable salt thereof, is administered once daily, and provides a treprostinil Cₘₐₓ ranging from about 80 pg/mL to about 885 pg/mL, for example, about 80 pg/mL, about 90 pg/mL, about 100 pg/mL, about 110 pg/mL, about 120 pg/mL, about 130 pg/mL, about 135 pg/mL, about 140 pg/mL, about 140 pg/mL, about 150 pg/mL, about 160 pg/mL, about 170 pg/mL, about 180 pg/mL, about 190 pg/mL, about 200 pg/mL, about 210 pg/mL, about 220 pg/mL, about 230 pg/mL, about 240 pg/mL, about 250 pg/mL, about 260 pg/mL, about 270 pg/mL, about 280 pg/mL, about 290 pg/mL, about 300 pg/mL, about 310 pg/mL, about 320 pg/mL, about 330 pg/mL, about 340 pg/mL, about 350 pg/mL, about 360 pg/mL, about 370 pg/mL, about 380 pg/mL, about 390 pg/mL, about 400 pg/mL, about 410 pg/mL, about 420 pg/mL, about 430 pg/mL, about 440 pg/mL, about 450 pg/mL, about 460 pg/mL, about 470 pg/mL, about 480 pg/mL, about 490 pg/mL, about 500 pg/mL, about 510 pg/mL, about 520 pg/mL, about 530 pg/mL, about 540 pg/mL, about 550 pg/mL, about 560 pg/mL, about 570 pg/mL, about 580 pg/mL, about 590 pg/mL, about 600 pg/mL, about 610 pg/mL, about 620 pg/mL, about 630 pg/mL, about 640 pg/mL, about 650 pg/mL, about 660 pg/mL, about 670 pg/mL, about 680 pg/mL, about 690 pg/mL, and about 700 pg/mL, about 710 pg/mL, about 720 pg/mL, about 730 pg/mL, about 740 pg/mL, about 750 pg/mL, about 760 pg/mL, about 770 pg/mL, about 780 pg/mL, about 790 pg/mL, about 800 pg/mL, about 810 pg/mL, about 820 pg/mL, about 830 pg/mL, about 840 pg/mL, about 850 pg/mL, about 860 pg/mL, about 870 pg/mL, or about 880 pg/mL, including all values and ranges therein. In some embodiments, about 400 μg of the compound of Formula (II), or enantiomer or diastereomer thereof, or pharmaceutically acceptable salt thereof, is administered once daily and provides a treprostinil Cₘₐₓ from about 80%-125% of a range of about 100 pg/mL to about 705 pg/mL. In some embodiments, about 400 μg of the compound of Formula (II), or enantiomer or diastereomer thereof, or pharmaceutically acceptable salt thereof, is administered once daily and provides a treprostinil Cₘₐₓ from about 80%-125% of a range from about 200 pg/mL to about 605 pg/mL.

In some embodiments, about 450 μg of the compound of Formula (II) or enantiomer or diastereomer thereof, or pharmaceutically acceptable salt thereof, and provides a treprostinil Cₘₐₓ ranging from about 80% to about 125% of about 387 (38.6) pg/mL.

In some embodiments, about 480 μg of the compound of Formula (II), or enantiomer or diastereomer thereof, or pharmaceutically acceptable salt thereof, is administered once daily, and provides a treprostinil Cₘₐₓ ranging from about 95 pg/mL to about 1350 pg/mL, for example, about 95 pg/mL, about 100 pg/mL, about 110 pg/mL, about 120 pg/mL, about 130 pg/mL, about 135 pg/mL, about 140 pg/mL, about 140 pg/mL, about 150 pg/mL, about 160 pg/mL, about 170 pg/mL, about 180 pg/mL, about 190 pg/mL, about 200 pg/mL, about 210 pg/mL, about 220 pg/mL, about 230 pg/mL, about 240 pg/mL, about 250 pg/mL, about 260 pg/mL, about 270 pg/mL, about 280 pg/mL, about 290 pg/mL, about 300 pg/mL, about 310 pg/mL, about 320 pg/mL, about 330 pg/mL, about 340 pg/mL, about 350 pg/mL, about 360 pg/mL, about 370 pg/mL, about 380 pg/mL, about 390 pg/mL, about 400 pg/mL, about 410 pg/mL, about 420 pg/mL, about 430 pg/mL, about 440 pg/mL, about 450 pg/mL, about 460 pg/mL, about 470 pg/mL, about 480 pg/mL, about 490 pg/mL, about 500 pg/mL, about 510 pg/mL, about 520 pg/mL, about 530 pg/mL, about 540 pg/mL, about 550 pg/mL, about 560 pg/mL, about 570 pg/mL, about 580 pg/mL, about 590 pg/mL, about 600 pg/mL, about 610 pg/mL, about 620 pg/mL, about 630 pg/mL, about 640 pg/mL, about 650 pg/mL, about 660 pg/mL, about 670 pg/mL, about 680 pg/mL, about 690 pg/mL, and about 700 pg/mL, about 710 pg/mL, about 720 pg/mL, about 730 pg/mL, about 740 pg/mL, about 750 pg/mL, about 760 pg/mL, about 770 pg/mL, about 780 pg/mL, about 790 pg/mL, about 800 pg/mL, about 810 pg/mL, about 820 pg/mL, about 830 pg/mL, about 840 pg/mL, about 850 pg/mL, about 860 pg/mL, about 870 pg/mL, about 880 pg/mL, about 890 pg/mL, about 900 pg/mL, about 910 pg/mL, about 920 pg/mL, about 930 pg/mL, about 940 pg/mL, about 950 pg/mL, about 960 pg/mL, about 970 pg/mL, about 980 pg/mL, about 1000 pg/mL, about 1010 pg/mL, about 1020 pg/mL, about 1030 pg/mL, about 1040 pg/mL, about 1050 pg/mL, about 1060 pg/mL, about 1065 pg/mL, about 1070 pg/mL, about 1080 pg/mL, about 1090 pg/mL, about 1100 pg/mL, about 1110 pg/mL, about 1120 pg/mL, about 1130 pg/mL, about 1140 pg/mL, about 1150 pg/mL, about 1160 pg/mL, about 1170 pg/mL, about 1180 pg/mL, about 1190 pg/mL, about 1200 pg/mL, about 1210 pg/mL, about 1220 pg/mL, about 1230 pg/mL, about 1240 pg/mL, about 1250 pg/mL, about 1260 pg/mL, about 1270 pg/mL, about 1280 pg/mL, about 1290 pg/mL, about 1300 pg/mL, about 1310 pg/mL, about 1320 pg/mL, about 1330 pg/mL, about 1340 pg/mL, or about 1350 pg/mL including all values and ranges therein. In some embodiments, about 480 μg of the compound of Formula (II), or enantiomer or diastereomer thereof, or pharmaceutically acceptable salt thereof, is administered once daily and provides a treprostinil Cₘₐₓ from about 80%-125% of about 120 μg/mL to about 855 pg/mL. In some embodiments, about 480 μg of the compound of Formula (II), or enantiomer or diastereomer thereof, or pharmaceutically acceptable salt thereof, is administered once daily and provides a treprostinil Cₘₐₓ from about 80%-125% of a range from about 240 pg/mL to about 730 pg/mL. In some embodiments, about 480 μg of the compound of Formula (II), or enantiomer or diastereomer thereof, or pharmaceutically acceptable salt thereof, is administered once daily and provides a treprostinil Cₘₐₓ of about 1246 μg/mL.

In some embodiments, about 640 μg of the compound of Formula (II), or enantiomer or diastereomer thereof, or pharmaceutically acceptable salt thereof, is administered once daily, and provides a treprostinil Cₘₐₓ ranging from about 130 pg/mL to about 1650 pg/mL, for example, about 130 pg/mL, about 135 pg/mL, about 140 pg/mL, about 140 pg/mL, about 150 pg/mL, about 160 pg/mL, about 170 pg/mL, about 180 pg/mL, about 190 pg/mL, about 200 pg/mL, about 210 pg/mL, about 220 pg/mL, about 230 pg/mL, about 240 pg/mL, about 250 pg/mL, about 260 pg/mL, about 270 pg/mL, about 280 pg/mL, about 290 pg/mL, about 300 pg/mL, about 310 pg/mL, about 320 pg/mL, about 330 pg/mL, about 340 pg/mL, about 350 pg/mL, about 360 pg/mL, about 370 pg/mL, about 380 pg/mL, about 390 pg/mL, about 400 pg/mL, about 410 pg/mL, about 420 pg/mL, about 430 pg/mL, about 440 pg/mL, about 450 pg/mL, about 460 pg/mL, about 470 pg/mL, about 480 pg/ml, about 490 pg/mL, about 500 pg/mL, about 510 pg/mL, about 520 pg/mL, about 530 pg/mL, about 540 pg/mL, about 550 pg/mL, about 560 pg/mL, about 570 pg/mL, about 580 pg/mL, about 590 pg/mL, about 600 pg/mL, about 610 pg/mL, about 620 pg/mL, about 630 pg/mL, about 640 pg/mL, about 650 pg/mL, about 660 pg/mL, about 670 pg/mL, about 680 pg/mL, about 690 pg/mL, and about 700 pg/mL, about 710 pg/mL, about 720 pg/mL, about 730 pg/mL, about 740 pg/mL, about 750 pg/mL, about 760 pg/mL, about 770 pg/mL, about 780 pg/mL, about 790 pg/mL, about 800 pg/mL, about 810 pg/mL, about 820 pg/mL, about 830 pg/mL, about 840 pg/mL, about 850 pg/mL, about 860 pg/mL, about 870 pg/mL, about 880 pg/mL, about 890 pg/mL, about 900 pg/mL, about 910 pg/mL, about 920 pg/mL, about 930 pg/mL, about 940 pg/mL, about 950 pg/mL, about 960 pg/mL, about 970 pg/mL, about 980 pg/mL, about 1000 pg/mL, about 1010 pg/mL, about 1020 pg/mL, about 1030 pg/mL, about 1040 pg/mL, about 1050 pg/mL, about 1060 pg/mL, about 1070 pg/mL, about 1080 pg/mL, about 1090 pg/mL, about 1100 pg/mL, about 1110 pg/mL, about 1120 pg/mL, about 1130 pg/mL, about 1140 pg/mL, about 1150 pg/mL, about 1160 pg/mL, about 1170 pg/mL, about 1180 pg/mL, about 1190 pg/mL, about 1200 pg/mL, about 1210 pg/mL, about 1220 pg/mL, about 1230 pg/n, about 1240 pg/mL, about 1250 pg/mL, about 1260 pg/mL, about 1270 pg/mL, about 1280 pg/mL, about 1290 pg/mL, about 1300 pg/mL, about 1310 pg/mL, about 1320 pg/mL, about 1330 pg/mL, about 1340 pg/mL, about 1350 pg/mL, about 1360 pg/mL, about 1370 pg/mL, about 1380 pg/mL, about 1390 pg/mL, about 1400 pg/mL, about 1410 pg/mL, about 1420 pg/mL, about 1430 pg/mL, about 1440 pg/mL, about 1450 pg/mL, about 1460 pg/mL, about 1470 pg/mL, about 1480 pg/mL, about 1490 pg/mL, about 1500 pg/mL, about 1510 pg/mL, about 1520 pg/mL, about 1530 pg/mL, about 1540 pg/mL, about 1550 pg/mL, about 1560 pg/mL, about 1570 pg/mL, about 1580 pg/mL, about 1590 pg/mL, about 1600 pg/mL, about 1610 pg/mL, about 1620 pg/mL, about 1630 pg/mL, about 1640 pg/mL, or about 1650 pg/mL including all values and ranges therein. In some embodiments, about 640 μg of the compound of Formula (II), or enantiomer or diastereomer thereof, or pharmaceutically acceptable salt thereof, is administered once daily and provides a treprostinil Cₘₐₓ from about 80%-125% of a range of about 160 pg/mL to about 1140 pg/mL. In some embodiments, about 640 μg of the compound of Formula (II), or enantiomer or diastereomer thereof, or pharmaceutically acceptable salt thereof, is administered once daily and provides a treprostinil Cₘₐₓ ranging from about 80%-125% of about 325 pg/mL to about 980 pg/mL. In some embodiments, about 640 μg of the compound of Formula (II), or enantiomer or diastereomer thereof, or pharmaceutically acceptable salt thereof, is administered once daily and provides a treprostinil Cₘₐₓ of about 1594 pg/mL.

In some embodiments, the about 675 μg of the compound of Formula (II), or enantiomer or diastereomer thereof, or pharmaceutically acceptable salt thereof, is administered, and provides a treprostinil Cₘₐₓ ranging from about 80% to about 125% of about 717 (52.8) pg/mL.

In some embodiments, the about 720 μg of the compound of Formula (II), or enantiomer or diastereomer thereof, or pharmaceutically acceptable salt thereof, is administered, and provides a treprostinil Cₘₐₓ ranging from about 80% to about 125% of about 185 μg/mL to about 1300 μg/mL, including all values and ranges therein. In some embodiments, about 720 pig of the compound of Formula (II), or enantiomer or diastereomer thereof, or pharmaceutically acceptable salt thereof, is administered, and provides a treprostinil Cₘₐₓ ranging from about 80% to about 125% of about 368 μg/mL to about 1105 μg/mL, including all values and ranges therein.

In some embodiments, about 800 μg of the compound of Formula (II), or enantiomer or diastereomer thereof, or pharmaceutically acceptable salt thereof, is administered, and provides a treprostinil Cₘₐₓ ranging from about 80% to about 125% of about 200 μg/ML to about 1430 μg/mL, including all values and ranges therein. In some embodiments, about 800 μg of the compound of Formula (II), or enantiomer or diastereomer thereof, or pharmaceutically acceptable salt thereof, is administered, and provides a treprostinil Cₘₐₓ ranging from about 80% to about 125% of about 400 μg/mL to about 1230 μg/mL, including all values and ranges therein.

In some embodiments, about 880 μg of the compound of Formula (II), or enantiomer or diastereomer thereof, or pharmaceutically acceptable salt thereof, is administered, and provides a treprostinil Cₘₐₓ ranging from about 80% to about 125% of about 225 μg/mL to about 1580 μg/mL, including all values and ranges therein. In some embodiments, about 800 pig of the compound of Formula (II), or enantiomer or diastereomer thereof, or pharmaceutically acceptable salt thereof, is administered, and provides a treprostinil Cₘₐₓ ranging from about 80% to about 125% of about 490 μg/mL to about 1480 μg/mL, including all values and ranges therein.

In some embodiments, about 960 μg of the compound of Formula (II), or enantiomer or diastereomer thereof, or pharmaceutically acceptable salt thereof, is administered, and provides a treprostinil Cₘₐₓ ranging from about 80% to about 125% of about 240 μg/mL to about 1720 μg/mL, including all values and ranges therein. In some embodiments, about 960 μg of the compound of Formula (II), or enantiomer or diastereomer thereof, or pharmaceutically acceptable salt thereof, is administered, and provides a treprostinil Cₘₐₓ ranging from about 80% to about 125% of about 490 μg/mL to about 1480 μg/mL, including all values and ranges therein.

In some embodiments, about 1040 μg of the compound of Formula (II), or enantiomer or diastereomer thereof, or pharmaceutically acceptable salt thereof, is administered, and provides a treprostinil Cₘₐₓ ranging from about 80% to about 125% of about 530 μg/mL to about 1605 μg/mL, including all values and ranges therein.

In some embodiments, about 1120 μg of the compound of Formula (II), or enantiomer or diastereomer thereof, or pharmaceutically acceptable salt thereof, is administered, and provides a treprostinil Cₘₐₓ ranging from about 80% to about 125% of about 280 μg/mL to about 2020 μg/mL, including all values and ranges therein. In some embodiments, about 1120 μg of the compound of Formula (II), or enantiomer or diastereomer thereof, or pharmaceutically acceptable salt thereof, is administered, and provides a treprostinil Cₘₐₓ ranging from about 80% to about 125% of about 575 μg/mL to about 1730 μg/mL, including all values and ranges therein.

In some embodiments, about 1200 μg of the compound of Formula (II), or enantiomer or diastereomer thereof, or pharmaceutically acceptable salt thereof, is administered, and provides a treprostinil Cₘₐₓ ranging from about 80% to about 125% of about 300 μg/mL to about 2165 μg/mL, including all values and ranges therein. In some embodiments, about 1200 μg of the compound of Formula (II), or enantiomer or diastereomer thereof, or pharmaceutically acceptable salt thereof, is administered, and provides a treprostinil Cₘₐₓ ranging from about 80% to about 125% of about 615 μg/mL to about 1855 μg/mL, including all values and ranges therein.

In some embodiments, about 1280 μg of the compound of Formula (II), or enantiomer or diastereomer thereof, or pharmaceutically acceptable salt thereof, is administered, and provides a treprostinil Cₘₐₓ ranging from about 80% to about 125% of about 325 μg/mL to about 2310 μg/mL, including all values and ranges therein. In some embodiments, about 1280 μg of the compound of Formula (II), or enantiomer or diastereomer thereof, or pharmaceutically acceptable salt thereof, is administered, and provides a treprostinil Cₘₐₓ ranging from about 80% to about 125% of about 660 μg/mL to about 1980 μg/mL, including all values and ranges therein.

In some embodiments, about 80 μg of the compound of Formula (II), or enantiomer or diastereomer thereof, or pharmaceutically acceptable salt thereof, is administered and provides a treprostinil AUC_{0-inf} ranging from about 375 μg*h/mL to about 1800 μg*h/mL, for example, 375 μg*h/mL, 400 μg*h/mL, 500 μg*h/mL, 600 μg*h/mL, about 700 μg*h/mL, about 800 μg*h/mL, about 900 μg*h/mL, about 1000 μg*h/mL, about 1100 μg*h/mL, about 1200 μg*h/mL, about 1300 μg*h/mL, about 1400 μg*h/mL, about 1500 μg*h/mL, about 1600 μg*h/mL, about 1700 μg*h/mL, or about 1800 μg*h/mL, including all values and ranges therein. In some embodiments, about 80 μg of the compound of Formula (II), is administered once daily and provides a treprostinil AUC_{0-inf} from about 80%-125% of a range of about 475 μg*h/mL to about 1430 μg*h/mL. In some embodiments, about 80 μg of the compound of Formula (II), or enantiomer or diastereomer thereof, or pharmaceutically acceptable salt thereof, is administered and provides a treprostinil AUC_{0-inf} from about 80%-125% of about 660 μg*h/mL to about 1240 μg*h/mL.

In some embodiments, about 112.5 μg of the compound of Formula (II), or enantiomer or diastereomer thereof, or pharmaceutically acceptable salt thereof, is administered and provides a treprostinil AUC_{0-inf} ranging from about 80% to about 125% of about 1090 (91.8) pg*hr/mL.

In some embodiments, about 160 μg of the compound of Formula (II), or enantiomer or diastereomer thereof, or pharmaceutically acceptable salt thereof, is administered and provides a treprostinil AUC_{0-inf} ranging from about 630 pg*h/mL to about 3000 pg*h/mL, for example, 630 pg*h/mL, about 700 pg*h/mL, about 800 pg*h/mL, about 900 pg*h/mL, about 1000 pg*h/mL, about 1100 pg*h/mL, about 1200 pg*h/mL, about 1300 pg*h/mL, about 1400 pg*h/mL, about 1500 pg*h/mL, about 1600 pg*h/mL, about 1700 pg*h/mL, about 1800 pg*h/mL, about 1900 pg*h/mL, about 2000 pg*h/mL, about 2100 pg*h/mL, about 2200 pg*h/mL, about 2300 pg*h/mL, about 2400 pg*h/mL, about 2500 pg*h/mL, about 2600 pg*h/mL, about 2700 pg*h/mL, about 2800 pg*h/mL, about 2900 pg*h/mL, or about 3000 pg*h/mL, including all values and ranges therein. In some embodiments, about 160 μg of the compound of Formula (II), or enantiomer or diastereomer thereof, or pharmaceutically acceptable salt thereof, is administered and provides a treprostinil AUC_{0-inf} from about 80%-125% of a range from about 785 μg*h/mL to about 2370 μg*h/mL. In some embodiments, about 160 μg of the compound of Formula (II), or enantiomer or diastereomer thereof, or pharmaceutically acceptable salt thereof, is administered and provides a treprostinil AUC_{0-inf} from about 80%-125% of a range from about 1100 μg*h/mL to about 2050 μg*h/mL.

In some embodiments, the about 225 μg of a compound of Formula (II), or enantiomer or diastereomer thereof, or pharmaceutically acceptable salt thereof, is administered and provides an AUC_{0-inf} ranging from about 80% to about 125% of about 2130 (30.0) ng*h/mL. In some embodiments, about 225 gg of the compound of Formula (II) is administered and provides a steady state treprostinil AUC₀₋₂₄ (CV %) ranging from about 80% to about 125% of about 1680 (28.7) ng*h/mL. In some embodiments, about 225 μg of the compound of Formula (II) or enantiomer or diastereomer thereof, or pharmaceutically acceptable salt thereof, is administered and provides a steady state treprostinil AUC₀₋₂₄ (CV %) ranging from about 80% to about 125% of about 1790 (39.6) ng*h/mL.

In some embodiments, about 450 μg of the compound of Formula (II), or enantiomer or diastereomer thereof, or pharmaceutically acceptable salt thereof, is administered and provides a treprostinil AUC_{0-inf} ranging from about 80% to about 125% of about 4040 (27.4) pg*hr/mL.

In some embodiments, about 240 μg of the compound of Formula (II), or enantiomer or diastereomer thereof, or pharmaceutically acceptable salt thereof, is administered and provides a treprostinil AUC_{0-inf} ranging from about 880 pg*h/mL to about 4130 pg*h/mL, for example, about 800 pg*h/mL, about 900 pg*h/mL, about 950 pg*h/mL, about 1000 pg*h/mL, about 1050 pg*h/mL, about 1100 pg*h/mL, about 1150 pg*h/mL, about 1200 pg*h/mL, about 1250 pg*h/mL, about 1300 pg*h/mL, about 1350 pg*h/mL, about 1400 pg*h/mL, about 1450 pg*h/mL, about 1500 pg*h/mL, about 1550 pg*h/mL, about 1600 pg*h/mL, about 1650 pg*h/mL, about 1700 pg*h/mL, about 1750 pg*h/mL, about 1800 pg*h/mL, about 1850 pg*h/mL, about 1950 pg*h/mL, about 2000 pg*h/mL, about 2050 pg*h/mL, about 2100 pg*h/mL, about 2150 pg*h/mL, about 2200 pg*h/mL, about 2250 pg*h/mL, about 2300 pg*h/mL, about 2350 pg*h/mL, about 2400 pg*h/mL, about 2450 pg*h/mL, about 2500 pg*h/mL, about 2550 pg*h/mL, about 2600 pg*h/mL, about 2650 pg*h/mL, about 2700 pg*h/mL, about 2750 pg*h/mL, about 2800 pg*h/mL, about 2850 pg*h/mL, about 2950 pg*h/mL, about 3000 pg*h/mL, about 3050 pg*h/mL, about 3100 pg*h/mL, about 3150 pg*h/mL, about 3200 pg*h/mL, about 3250 pg*h/mL, about 3300 pg*h/mL, about 3350 pg*h/mL, about 3400 pg*h/mL, about 3450 pg*h/mL, about 3500 pg*h/mL, about 3550 pg*h/mL, about 3600 pg*h/mL, about 3650 pg*h/mL, about 3700 pg*h/mL, about 3750 pg*h/mL, about 3800 pg*h/mL, about 3850 pg*h/mL, about 3950 pg*h/mL, about 4000 pg*h/mL, about 4050 pg*h/mL, about 4100 pg*h/mL, about 4130 pg*h/mL, including all values and ranges therein. In some embodiments, about 240 μg of the compound of Formula (II), or enantiomer or diastereomer thereof, or pharmaceutically acceptable salt thereof, is administered and provides a treprostinil AUC_{0-inf} from about 80%-125% of a range from about 1100 μg*h/mL to about 3305 μg*h/mL, In some embodiments, about 240 μg of the compound of Formula (1I), or enantiomer or diastereomer thereof, or pharmaceutically acceptable salt thereof, is administered and provides a treprostinil AUC_{0-inf} from about 80%-125% of a range from about 1540 μg*h/mL to about 2865 μg*h/mL.

In some embodiments, about 320 μg of the compound of Formula (II), or enantiomer or diastereomer thereof, or pharmaceutically acceptable salt thereof, is administered and provides a treprostinil AUC_{0-inf} ranging from about 1130 pg*h/mL to about 5310 pg*h/mL, for example, about 1130 pg*h/mL, about 1200 pg*h/mL, about 1300 pg*h/mL, about 1400 pg*h/mL, about 1450 pg*h/mL, about 1500 pg*h/mL, about 1550 pg*h/mL, about 1600 pg*h/mL, about 1700 pg*h/m L, about 1800 pg*h/mL, about 1900 pg*h/mL, about 2000 pg*h/mL, about 2100 pg*h/mL, about 2200 pg*h/mL, about 2300 pg*h/mL, about 2400 pg*h/mL, about 2500 pg*h/mL, about 2600 pg*h/mL, about 2700 pg*h/mL, about 2800 pg*h/mL, about 2900 pg*h/mL, about 3000 pg*h/mL, about 3100 pg*h/mL, about 3200 pg*h/mL, about 3300 pg*h/mL, about 3400 pg*h/mL, about 3500 pg*h/mL, about 3600 pg*h/mL, about 3700 pg*h/mL, about 3800 pg*h/mL, about 3900 pg*h/mL, about 4000 pg*h/mL, about 4100 pg*h/mL, about 4200 pg*h/mL, about 4300 pg*h/mL, about 4400 pg*h/mL, about 4500 pg*h/mL, about 4600 pg*h/mL, about 4700 pg*h/mL, about 4800 pg*h/mL, about 4900 pg*h/mL, about 5000 pg*h/mL, about 5100 pg*h/mL, about 5200 pg*h/mL, about 5300 pg*h/mL, about 5300 pg*h/mL, or about 5310 pg*h/mL, including all values and ranges therein. In some embodiments, about 320 μg of the compound of Formula (II), or enantiomer or diastereomer thereof, or pharmaceutically acceptable salt thereof, is administered provides a treprostinil AUC_{0-inf} from about 80%-125% of a range from about 1400 μg*h/mL to about 4250 μg*h/mL, In some embodiments, about 320 pig of the compound of Formula (II), or enantiomer or diastereomer thereof, or pharmaceutically acceptable salt thereof, is administered once daily and provides a treprostinil AUC_{0-inf} from about 80%-125% of a range from about 1975 μg*h/mL to about 3680 μg*h/mL.

In some embodiments, about 400 μg of the compound of Formula (II), or enantiomer or diastereomer thereof, or pharmaceutically acceptable salt thereof, is administered and provides a treprostinil AUC_{0-inf} ranging from about 1380 pg*h/mL to about 6480 pg*h/mL, for example, about 1380 pg*h/mL, about 1400 pg*h/mL, about 1450 pg*h/mL, about 1500 pg*h/mL, about 1550 pg*h/mL, about 1600 pg*h/mL, about 1700 pg*h/mL, about 1800 pg*h/mL, about 1900 pg*h/mL, about 2000 pg*h/mL, about 2100 pg*h/mL, about 2200 pg*h/mL, about 2300 pg*h/mL, about 2400 pg*h/mL, about 2500 pg*h/mL, about 2600 pg*h/mL, about 2700 pg*h/mL, about 2800 pg*h/mL, about 2900 pg*h/mL, about 3000 pg*h/mL, about 3100 pg*h/mL, about 3200 pg*h/mL, about 3300 pg*h/mL, about 3400 pg*h/mL, about 3500 pg*h/mL, about 3600 pg*h/mL, about 3700 pg*h/mL, about 3800 pg*h/mL, about 3900 pg*h/mL, about 4000 pg*h/mL, about 4100 pg*h/mL, about 4200 pg*h/mL, about 4300 pg*h/mL, about 4400 pg*h/mL, about 4500 pg*h/mL, about 4600 pg*h/mL, about 4700 pg*h/mL, about 4800 pg*h/mL, about 4900 pg*h/mL, about 5000 pg*h/mL, about 5100 pg*h/mL, about 5200 pg*h/mL, about 5300 pg*h/mL, about 5400 pg*h/mL, about 5500 pg*h/mL, about 5600 pg*h/mL, about 5700 pg*h/mL, about 5800 pg*h/mL, about 5900 pg*h/mL, about 6000 pg*h/mL, about 6100 pg*h/mL, about 6200 pg*h/mL, about 6300 pg*h/mL, about 6400 pg*h/mL, or about 6480 pg*h/mL, including all values and ranges therein. In some embodiments, about 400 μg of the compound of Formula (II), or enantiomer or diastereomer thereof, or pharmaceutically acceptable salt thereof, is administered and provides a treprostinil AUC_{0-inf} from about 80%-125% of a range from about 1725 μg*h/mL to about 5180 μg*h/mL. In some embodiments, about 400 μg of the compound of Formula (II), or enantiomer or diastereomer thereof, or pharmaceutically acceptable salt thereof, is administered and provides a treprostinil AUC_{0-inf} from about 80%-125% of a range from about 2415 pg*hr/mL to about 4490 μg*h/mL.

In some embodiments, about 480 μg of the compound of Formula (II), or enantiomer or diastereomer thereof, or pharmaceutically acceptable salt thereof, is administered and provides a treprostinil AUC_{0-inf} ranging from about 1630 pg*h/mL to about 7650 pg*h/mL, for example, about 1630 pg*h/mL, about 1700 pg*h/mL, about 1800 pg*h/mL, about 1900 pg*h/mL, about 2000 pg*h/mL, about 2100 pg*h/mL, about 2200 pg*h/mL, about 2300 pg*h/mL, about 2400 pg*h/mL, about 2500 pg*h/mL, about 2600 pg*h/mL, about 2700 pg*h/mL, about 2800 pg*h/mL, about 2900 pg*h/mL, about 3000 pg*h/mL, about 3100 pg*h/mL, about 3200 pg*h/mL, about 3300 pg*h/mL, about 3400 pg*h/mL, about 3500 pg*h/mL, about 3600 pg*h/mL, about 3700 pg*h/mL, about 3800 pg*h/mL, about 3900 pg*h/mL, about 4000 pg*h/mL, about 4100 pg*h/mL, about 4200 pg*h/mL, about 4300 pg*h/mL, about 4400 pg*h/mL, about 4500 pg*h/mL, about 4600 pg*h/mL, about 4700 pg*h/mL, about 4800 pg*h/mL, about 4900 pg*h/mL, about 5000 pg*h/mL, about 5100 pg*h/mL, about 5200 pg*h/mL, about 5300 pg*h/mL, about 5400 pg*h/mL, about 5500 pg*h/mL, about 5600 pg*h/mL, about 5700 pg*h/mL, about 5800 pg*h/mL, about 5900 pg*h/mL, about 6000 pg*h/mL, about 6100 pg*h/mL, about 6200 pg*h/mL, about 6300 pg*h/mL, about 6400 pg*h/mL, about 6500 pg*h/mL, about 6600 pg*h/mL, about 6700 pg*h/mL, about 6800 pg*h/mL, about 6900 pg*h/mL, about 7000 pg*h/mL, about 7100 pg*h/mL, about 7200 pg*h/mL, about 7300 pg*h/mL, about 7400 pg*h/mL, about 7500 pg*h/mL, or about 7650 pg*h/mL, including all values and ranges therein. In some embodiments, about 480 pg of the compound of Formula (II), or enantiomer or diastereomer thereof, or pharmaceutically acceptable salt thereof, is administered and provides a treprostinil AUC_{0-inf} from about 80%-125% of a range from about 2040 μg*h/mL to about 6120 μg*h/mL. In some embodiments, about 480 pig of the compound of Formula (II), or enantiomer or diastereomer thereof, or pharmaceutically acceptable salt thereof, is administered and provides a treprostinil AUC_{0-inf} from about 80%-125% of a range from about 2855 μg*h/mL to about 5310 μg*h/mL.

In some embodiments, about 640 μg of the compound of Formula (II), or enantiomer or diastereomer thereof, or pharmaceutically acceptable salt thereof, is administered and provides a treprostinil AUC_{0-inf} ranging from about 2130 pg*h/mL to about 10000 pg*h/mL, for example, about 2130 pg*h/mL, about 2200 pg*h/mL, about 2300 pg*h/mL, about 2400 pg*h/mL, about 2500 pg*h/mL, about 2600 pg*h/mL, about 2700 pg*h/mL, about 2800 pg*h/mL, about 2900 pg*h/mL, about 3000 pg*h/mL, about 3100 pg*h/mL, about 3200 pg*h/mL, about 3300 pg*h/mL, about 3400 pg*h/mL, about 3500 pg*h/mL, about 3600 pg*h/mL, about 3700 pg*h/mL, about 3800 pg*h/mL, about 3900 pg*h/mL, about 4000 pg*h/mL, about 4100 pg*h/mL, about 4200 pg*h/m L, about 4300 pg*h/mL, about 4400 pg*h/mL, about 4500 pg*h/mL, about 4600 pg*h/mL, about 4700 pg*h/mL, about 4800 pg*h/mL, about 4900 pg*h/mL, about 5000 pg*h/mL, about 5100 pg*h/mL, about 5200 pg*h/mL, about 5300 pg*h/mL, about 5400 pg*h/mL, about 5500 pg*hr/mL, about 5600 pg*h/mL, about 5700 pg*h/mL, about 5800 pg*h/mL, about 5900 pg*h/mL, about 6000 pg*h/mL, about 6100 pg*h/mL, about 6200 pg*h/mL, about 6300 pg*h/mL, about 6400 pg*h/mL, about 6500 pg*h/mL, about 6600 pg*h/mL, about 6700 pg*h/mL, about 6800 pg*h/mL, about 6900 pg*h/mL, about 7000 pg*h/mL, about 7100 pg*h/mL, about 7200 pg*h/mL, about 7300 pg*h/mL, about 7400 pg*h/mL, about 7500 pg*h/mL, about 7600 pg*h/mL, about 7700 pg*h/mL, about 7800 pg*h/mL, about 8000 pg*h/mL, about 8100 pg*h/mL, about 8200 pg*h/mL, about 8300 pg*h/mL, about 8400 pg*h/mL, about 8500 pg*h/mL, about 8600 pg*h/mL, about 8700 pg*h/mL, about 8800 pg*h/mL, about 8900 pg*h/mL, about 9000 pg*h/mL, about 9100 pg*h/mL, about 9200 pg*h/mL, about 9300 pg*h/mL, about 9350 pg*h/mL, about 9400 pg**h/mL, about 9450 pg*h/mL, about 9500 pg*h/mL, about 9600 pg*h/mL, about 9700 pg*h/mL, about 9800 pg*h/mL, about 9900 pg*h/mL, or about 10000 pg*h/mL, including all values and ranges therein. In some embodiments, about 640 μg of the compound of Formula (II), or enantiomer or diastereomer thereof, or pharmaceutically acceptable salt thereof, is administered and provides a treprostinil AUC_{0-inf} from about 80%-125% of a range from about 2650 μg*h/mL to about 8000 μg*h/mL. In some embodiments, about 640 jpg of the compound of Formula (II), or enantiomer or diastereomer thereof, or pharmaceutically acceptable salt thereof, is administered and provides a treprostinil AUC_{0-inf} from about 80%-125% of a range from about 3730 to about 6935 μg*h/mL.

In some embodiments, about 675 μg of the compound of Formula (II), or enantiomer or diastereomer thereof, or pharmaceutically acceptable salt thereof, or a, is administered and provides a treprostinil AUC₀₋₂₄ ranging from about 80% to about 125% of about 5480 (13.8) pg*hr/mL. In a further embodiment, the compound is a compound of Formula (II).

In some embodiments, about 720 μg of the compound of Formula (II), or enantiomer or diastereomer thereof, or pharmaceutically acceptable salt thereof, is administered and provides a treprostinil AUC_{0-inf} from about 80%-125% of a range from about 2975 μg*h/mL to about 8940 μg*h/mL. In some embodiments, about 720 μg of the compound of Formula (II), or enantiomer or diastereomer thereof, or pharmaceutically acceptable salt thereof, is administered and provides a treprostinil AUC_{0-inf} from about 80%-125% of a range from about 4170 μg*h/mL to about 7750 μg*h/mL.

In some embodiments, about 800 μg of the compound of Formula (II), or enantiomer or diastereomer thereof, or pharmaceutically acceptable salt thereof, is administered and provides a treprostinil AUC_{0-inf} from about 80%-125% of a range from about 3290 μg*h/mL to about 9900 μg*h/mL. In some embodiments, about 800 μg of the compound of Formula (II), or enantiomer or diastereomer thereof, or pharmaceutically acceptable salt thereof, is administered and provides a treprostinil AUC_{0-inf} from about 80%-125% of a range from about 4600 μg*h/mL to about 8560 μg*h/mL.

In some embodiments, about 880 μg of the compound of Formula (II), or enantiomer or diastereomer thereof, or pharmaceutically acceptable salt thereof, is administered and provides a treprostinil AUC_{0-inf} from about 80%-125% of a range from about 3605 μg*h/mL to about 10815 μg*h/mL. In some embodiments, about 880 μg of the compound of Formula (II), or enantiomer or diastereomer thereof, or pharmaceutically acceptable salt thereof, is administered and provides a treprostinil AUC_{0-inf} from about 80%-125% of a range from about 5045 μg*h/mL to about 9380 μg*h/mL.

In some embodiments, about 960 μg of the compound of Formula (II), or enantiomer or diastereomer thereof, or pharmaceutically acceptable salt thereof, is administered and provides a treprostinil AUC_{0-inf} from about 80%-125% of a range from about 3900 μg*h/mL to about 11800 μg*h/mL. In some embodiments, about 960 μg of the compound of Formula (II), or enantiomer or diastereomer thereof, or pharmaceutically acceptable salt thereof, is administered and provides a treprostinil AUC_{0-inf} from about 80%-125% of a range from about 5485 μg*h/mL to about 10190 μg*h/mL.

In some embodiments, about 1040 μg of the compound of Formula (II), or enantiomer or diastereomer thereof, or pharmaceutically acceptable salt thereof, is administered and provides a treprostinil AUC_{0-inf} from about 80%-125% of a range from about 4230 μg*h/mL to about 12700 μg*h/mL. In some embodiments, about 1040 μg of the compound of Formula (II), or enantiomer or diastereomer thereof, or pharmaceutically acceptable salt thereof, is administered and provides a treprostinil AUC-inf from about 80%-125% of a range from about 5920 μg*h/mL to about 11000 μg*h/mL.

In some embodiments, about 1120 μg of the compound of Formula (II), or enantiomer or diastereomer thereof, or pharmaceutically acceptable salt thereof, is administered and provides a treprostinil AUC_{0-inf} from about 80%-125% of a range from about 4500 μg*h/mL to about 13650 μg*h/mL, In some embodiments, about 1120 μg of the compound of Formula (II), or enantiomer or diastereomer thereof, or pharmaceutically acceptable salt thereof, is administered and provides a treprostinil AUC_{0-inf} from about 80%-125% of a range from about 6360 μg*h/mL to about 11815 μg*h/mL.

In some embodiments, about 1200 μg of the compound of Formula (II), or enantiomer or diastereomer thereof, or pharmaceutically acceptable salt thereof, is administered and provides a treprostinil AUC_{0-inf} from about 80%-125% of a range from about 4850 μg*h/mL to about 14600 μg*h/mL. In some embodiments, about 1200 μg of the compound of Formula (II), or enantiomer or diastereomer thereof, or pharmaceutically acceptable salt thereof, is administered and provides a treprostinil AUC_{0-inf} from about 80%-125% of a range from about 6795 μg*h/mL to about 12630 μg*h/m L.

In some embodiments, about 1280 μg of the compound of Formula (II), or enantiomer or diastereomer thereof, or pharmaceutically acceptable salt thereof, is administered and provides a treprostinil AUC_{0-inf} from about 80%-125% of a range from about 5150 μg*h/mL to about 15550 μg*h/mL. In some embodiments, about 1280 μg of the compound of Formula (II) is administered and provides a treprostinil AUC_{0-inf} from about 80%-125% of a range from about 7235 μg*h/mL to about 13445 μg*h/mL.

In some embodiments, about 80 pig to about 1280 μg of the compound of Formula (II), or enantiomer or diastereomer thereof, or pharmaceutically acceptable salt thereof, is administered once daily and provides a treprostinil plasma trough concentration ranging from about 3 pg/mL to about 250 mg/mL, for example about 4 pg/mL, about 4 pg/mL, about 5 pg/mL, about 10 pg/mL, about 15 pg/mL, about 20 pg/mL, about 25 pg/mL about 30 pg/mL, about 35 pg/mL, about 40 pg/mL, about 45 pg/mL, about 50 pg/mL, about 55 pg/mL, about 60 pg/mL, about 65 pg/mL, about 70 pg/mL, about 75 pg/mL, about 80 pg/mL, about 85 pg/mL, about 90 pg/mL, about 95 pg/mL, about 100 pg/mL, about 100 pg/mL, about 105 pg/mL, about 110 pg/mL, about 115 pg/mL, about 120 pg/mL, about 125 pg/mL about 130 pg/mL, about 135 pg/mL, about 140 pg/mL, about 145 pg/mL, about 150 pg/mL, about 155 pg/mL, about 160 pg/mL, about 165 pg/mL, about 170 pg/mL, about 175 pg/mL, about 180 pg/mL, about 185 pg/mL, about 190 pg/mL, about 195 pg/mL, about 200 pg/mL, about 200 pg/mL, about 205 pg/mL, about 210 pg/mL, about 215 pg/mL, about 220 pg/mL, about 225 pg/mL about 230 pg/mL, about 235 pg/mL, about 240 pg/mL, about 245 pg/mL, or about 250 pg/mL, including all values and ranges therein.

In some embodiments, about 80 μg of the compound of Formula (II), or enantiomer or diastereomer thereof, or pharmaceutically acceptable salt thereof, is administered once daily and provides a treprostinil plasma trough concentration ranging from about 3 pg/mL to about 25 mg/mL, for example, about 3 pg/mL, about 4 pg/mL pg/mL, about 5 pg/mL, about 10 pg/mL, about 15 pg/mL, about 20 pg/mL, or about 25 pg/mL, including all values and ranges therein. In a further embodiment, the treprostinil plasma trough concentration ranges from about 6 μg/mL to about 18 mg/mL.

In some embodiments, about 112.5 μg of the compound of Formula (II), or enantiomer or diastereomer thereof, or pharmaceutically acceptable salt thereof, is administered once daily and provides a treprostinil plasma trough concentration ranging from about 4 pg/mL to about 30 mg/mL, for example about 4 pg/mL, about 5 pg/mL, about 10 pg/mL, about 15 pg/mL, about 20 pg/mL, about 25 pg/mL, or about 30 pg/mL, including all values and ranges therein.

In some embodiments, about 160 μg of the compound of Formula (II), or enantiomer or diastereomer thereof, or pharmaceutically acceptable salt thereof, is administered once daily and provides a treprostinil plasma trough concentration ranging from about 5 pg/mL to about 35 mg/mL, for example about 5 pg/mL, about 10 pg/mL, about 15 pg/mL, about 20 pg/mL, about 25 pg/mL, about 30 pg/mL, or about 35 pg/mL, including all values and ranges therein. In a further embodiment, the treprostinil plasma trough concentration ranges from about 10 pg/mL to about 30 mg/mL, or from 15 pg/mL to about 25 pg/mL.

In some embodiments, about 225 μg of the compound of Formula (II), or enantiomer or diastereomer thereof, or pharmaceutically acceptable salt thereof, is administered once daily and provides a treprostinil plasma trough concentration ranging from about 15 pg/mL to about 45 mg/mL, for example about 15 pg/mL, about 20 pg/mL, about 25 pg/mL, about 30 pg/mL, about 35 pg/mL, about 40 pg/mL, or about 45 pg/mL, including all values and ranges therein.

In some embodiments, about 240 pg of the compound of Formula (II), or enantiomer or diastereomer thereof, or pharmaceutically acceptable salt thereof, is administered once daily and provides a treprostinil plasma trough concentration ranging from about 7 pg/mL to about 50 mg/mL, for example about 7 pg/mL, about 10 pg/mL, about 15 pg/mL, about 20 pg/mL, about 25 pg/mL, about 30 pg/mL, about 35 pg/mL, about 40 pg/mL, about 45 pg/mL, or about 50 pg/mL, including all values and ranges therein. In some embodiments, the treprostinil plasma trough concentration ranges from about 15 pg/mL to about 50 mg/mL, or from 20 pg/mL to about 45 pg/mL.

In some embodiments, about 320 pg of the compound of Formula (II), or enantiomer or diastereomer thereof, or pharmaceutically acceptable salt thereof, is administered once daily and provides a treprostinil plasma trough concentration ranging from about 9 pg/mL to about 65 mg/mL, for example about 9 pg/mL, about 10 pg/mL, about 15 pg/mL, about 20 pg/mL, about 25 pg/mL, about 30 pg/mL, about 35 pg/mL, about 40 pg/mL, about 45 pg/mL, about 50 pg/mL, about 55 pg/mL, about 60 pg/mL, or about 65 pg/mL, including all values and ranges therein. In some embodiments, the treprostinil plasma trough concentration ranges from about 15 pg/mL to about 50 mg/mL, or from 20 pg/mL to about 45 pg/mL.

In some embodiments, about 400 pg of the compound of Formula (II), or enantiomer or diastereomer thereof, or pharmaceutically acceptable salt thereof, is administered once daily and provides a treprostinil plasma trough concentration ranging from about 10 pg/mL to about 80 mg/mL, for example about 10 pg/mL, about 15 pg/mL, about 20 pg/mL, about 25 pg/mL, about 30 pg/ML, about 35 pg/mL, about 40 pg/ML, about 45 pg/mL, about 50 pg/ML, about 55 pg/mL, about 60 pg/mL, about 65 pg/mL, about 70 pg/mL, about 75 pg/mL, or about 80 pg/mL including all values and ranges therein. In some embodiments, the treprostinil plasma trough concentration ranging from about 35 pg/mL to about 70 mg/mL, or from 40 pg/mL to about 65 pg/mL.

In some embodiments, about 480 pg of the compound of Formula (II), or enantiomer or diastereomer thereof, or pharmaceutically acceptable salt thereof, is administered once daily and provides a treprostinil plasma trough concentration ranging from about 13 pg/mL to about 95 mg/mL, for example about 13 pg/mL, about 15 pg/mL, about 20 pg/mL, about 25 pg/mL, about 30 pg/mL, about 35 pg/mL, about 40 pg/mL, about 45 pg/mL, about 50 pg/mL, about 55 pg/mL, about 60 pg/mL, about 65 pg/mL, about 70 pg/mL, about 75 pg/mL, about 80 pg/mL, about 85 pg/mL, about 90 pg/mL, about 95 pg/mL, including all values and ranges therein. In some embodiments, the treprostinil plasma trough concentration ranging from about 25 pg/mL to about 75 mg/mL, or from 30 pg/mL to about 70 pg/mL.

In some embodiments, about 640 pg of the compound of Formula (II), or enantiomer or diastereomer thereof, or pharmaceutically acceptable salt thereof, is administered once daily and provides a treprostinil plasma trough concentration ranging from about 15 pg/mL to about 125 mg/mL, for example about 15 pg/mL, about 20 pg/mL, about 25 pg/mL, about 30 pg/mL, about 35 pg/mL, about 40 pg/mL, about 45 pg/mL, about 50 pg/mL, about 55 pg/mL, about 60 pg/mL, about 65 pg/mL, about 70 pg/mL, about 75 pg/mL, about 80 pg/mL, about 85 pg/mL, about 90 pg/mL, about 95 pg/mL, about 100 pg/mL, about 105 pg/mL, about 110 pg/mL, about 115 pg/mL, about 120 pg/mL, or about 125 pg/mL, including all values and ranges therein. In some embodiments, the treprostinil plasma trough concentration ranging from about 35 pg/mL to about 100 mg/mL, or from 50 pg/mL to about 90 pg/mL.

In some embodiments, about 720 μg of the compound of Formula (II) or enantiomer or diastereomer thereof, or pharmaceutically acceptable salt thereof, is administered once daily and provides a treprostinil plasma trough concentration ranging from about 20 pg/mL to about 135 mg/mL, including all values and ranges therein.

In some embodiments, about 800 μg of the compound of Formula (II), or enantiomer or diastereomer thereof, or pharmaceutically acceptable salt thereof, is administered once daily and provides a treprostinil plasma trough concentration ranging from about 21 pg/mL to about 150 mg/mL, including all values and ranges therein.

In some embodiments, about 960 μg of the compound of Formula (II), or enantiomer or diastereomer thereof, or pharmaceutically acceptable salt thereof, is administered once daily and provides a treprostinil plasma trough concentration ranging from about 25 pg/mL to about 180 mg/mL, including all values and ranges therein.

In some embodiments, about 1040 μg of the compound of Formula (II), or enantiomer or diastereomer thereof, or pharmaceutically acceptable salt thereof, is administered once daily and provides a treprostinil plasma trough concentration ranging from about 28 pg/mL to about 195 mg/mL, including all values and ranges therein.

In some embodiments, about 1120 μg of the compound of Formula (II), or enantiomer or diastereomer thereof, or pharmaceutically acceptable salt thereof, is administered once daily and provides a treprostinil plasma trough concentration ranging from about 30 pg/mL to about 209 mg/mL, including all values and ranges therein.

In some embodiments, about 1200 μg of the compound of Formula (II), or enantiomer or diastereomer thereof, or pharmaceutically acceptable salt thereof, is administered once daily and provides a treprostinil plasma trough concentration ranging from about 32 pg/mL to about 224 mg/mL, including all values and ranges therein.

In some embodiments, about 1280 μg of the compound of Formula (II), or enantiomer or diastereomer thereof, or pharmaceutically acceptable salt thereof, is administered once daily and provides a treprostinil plasma trough concentration ranging from about 34 pg/mL to about 238 mg/mL, including all values and ranges therein.

### EXAMPLES

The present invention is further illustrated by reference to the following Examples. However, it should be noted that these Examples, like the embodiments described above, are illustrative and are not to be construed as restricting the scope of the invention in any way.

The following examples use a treprostinil palmitil inhalation powder (TPIP) formulation described in Table 6. The composition of the formulation is expressed in weight ratios, targeted weight percentages calculated based on the weight ratios, and actual weight percentages of the components from a typical batch of each formulation are summarized in Table 6.

TABLE 6
Exemplary TPIP composition

| | Quantity (% w) |
|---|---|
| TP (drug substance) | 1.0 |
| Leucine (dispersion | 29.3 |
| enhancer) | |
| Mannitol (bulking agent) | 69.7 |
| Filled Capsule Weight | 100 |

The actual wt % values shown are typical wt % values for the components in TPIP. Batches of TPIP with wt % for each component independently varying at or within ±5% of the typical wt % value as shown were observed to have equivalent properties and performance.

### Example 1: Study of Treprostinil Palmitil Inhalation Powder (TPIP) in Pulmonary Hypertension

Prior studies performed with TPIP over a range of doses revealed that the highest doses tested were well tolerated. Specifically, in a Phase I safety, tolerability, and PK study, healthy adults were administered single doses of 112.5 μg, 225 μg, 450 μg, and 675 μg of a compound of Formula (II). As shown in Table 7, all doses were determined to be safe and well-tolerated, and no serious or severe TEAEs were observed.

TABLE 7
TPIP single-dose groups (N = 26)

| | 112.5 μg | 225 μg | 450 μg | 675 μg | Placebo |
|---|---|---|---|---|---|
| | (n = 6) | (n = 6) | (n = 6) | (n = 6) | (n = 2) |
| Cough^{b} | 2 (33.3) | 2 (33.3) | 3 (50.0) | 4 (66.7) | 0 |
| Dizziness^{b} | 1 (16.7) | 1 (16.7) | 2 (33.3) | 3 (50.0) | 0 |
| Headache^{b} | 0 | 0 | 1 (16.7) | 1 (16.7) | 0 |
| Nausea^{b} | 0 | 1 (16.7) | 2 (33.3) | 1 (16.7) | 0 |
| Chest discomfort^{b} | 1 (16.7) | 0 | 1 (16.7) | 1 (16.7) | 0 |
| Throat irritation^{b} | 2 (33.3) | 2 (33.3) | 1 (16.7) | 0 | 0 |
| Hypotension^{b} | 0 | 1 (16.7) | 1 (16.7) | 2 (33.3) | 0 |
| Fatigue | 0 | 0 | 0 | 2 (33.3) | 0 |
| Feeling hot | 0 | 0 | 0 | 2 (33.3) | 0 |
| Hyperhidrosis | 0 | 0 | 0 | 2 (33.3) | 0 |

TPIP: treprostinil palmitil inhalation powder.
^{a}The safety population included all participants who were randomized and received ≥1 dose of assigned treatment.
^{b}AE of special interest.

In this Phase I study, an uptitration strategy was employed and was observed to improve tolerability. Specifically, 112.5 μg was administered on days 1-4, and then on day 5 the dose was increased to 225 μg, and the adverse event profile for the uptitration strategy was compared to subjects administered a single dose of 225 μg. As shown in Table 8, dose titration strategy improved tolerability.

TABLE 8
TPIP single-dose groups (N = 26)

| | | 112.5 μg QD + | |
|---|---|---|---|
| | 225 μg QD | 225 μg QD | Placebo |
| | (n = 6) | (n = 6) | (n = 4) |
| Cough^{b} | 6 (100.0) | 1 (16.7) | 2 (50.0) |
| Dizziness^{b} | 2 (33.3) | 1 (16.7) | 0 |
| Headache^{b} | 4 (66.7) | 2 (33.3) | 0 |
| Nausea^{b} | 3 (50.0) | 1 (16.7) | 0 |
| Chest discomfort^{b} | 2 (33.3) | 2 (33.3) | 0 |
| Throat irritation^{b} | 1 (16.7) | 0 | 0 |
| Hypotension^{b} | 0 | 0 | 0 |
| Fatigue | 0 | 0 | 0 |
| Feeling hot | 0 | 0 | 0 |
| Hyperhidrosis | 0 | 0 | 0 |

QD: once daily; TPIP: treprostinil palmitil inhalation powder.
^{a}The safety population included all participants who were randomized and received ≥1 dose of assigned treatment.
^{b}AE of special interest.

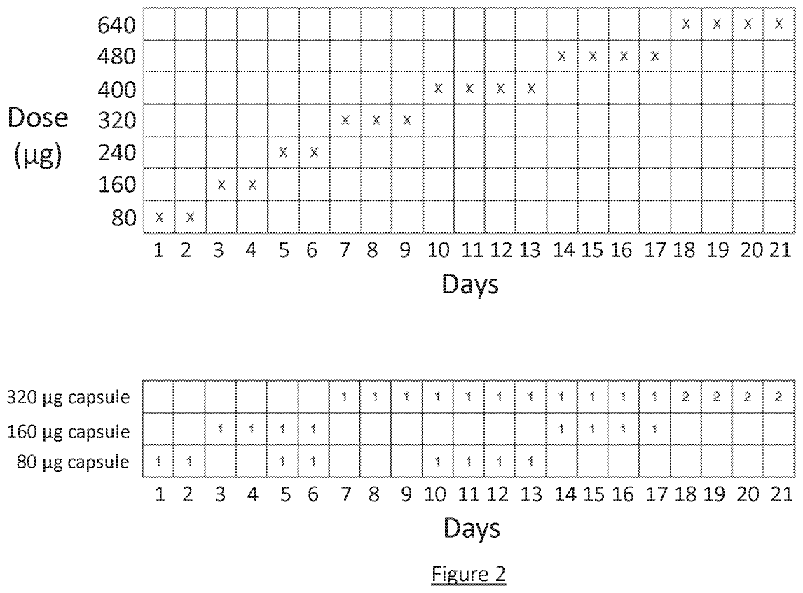

In a Phase 2 safety and efficacy study, the following doses of were investigated in subjects with pulmonary hypertension, including pulmonary arterial hypertension: 80 μg, 160 μg, 240 μg, 320 μg, 400 μg, 480 μg, and 640 μg. In this study, patients were administered a starting dose of 80 μg of the TPIP and the dose was titrated up to 640 μg according to FIG. 2.

All doses were well tolerated. While higher doses also displayed promising efficacy, certain subjects (e.g., patients with severe disease, disease progression, clinical worsening, or reduced efficacy of the dose that was previously effective) may benefit from higher doses. That all doses administered in these studies are well tolerated is surprising considering that the maximum single dose of TYVASO DPI©, a dry powder formulation of treprostinil, is 64 μg administered 4 times per day, for a maximum total daily dose of 256 μg. Based on these data, such subjects are titrated to a dose ranging from 700-1300 μg of treprostinil palmitil (which is equivalent to 445-826 μg free treprostinil).

### Example 2: Open Label Extension Stud to Evaluate the Safe Tolerability, and Effectiveness of the Long-Term Use of Treprostinil Palmitil Inhalation Powder (TPIP) in Participants with PAH

Background: due to the progressive nature of PAH, it is the usual course of care to continue patients with PAH on vasodilator medication, such as TPIP, indefinitely after initiation.

Primary Objective: to evaluate the safety and tolerability of the long-term use of TPIP in participants with PAH from studies INS1009-201, INS1009-202, and other lead-in studies of TPIP in participants with PAH.

### Secondary Objectives:

to evaluate the effect of the long-term use of TPIP on exercise capacity in participants with PAH;
to evaluate the effect of the long-term use of TPIP on blood biomarkers of disease severity in patients with PAH;
to evaluate the effect of the long-term use of TPIP on the mortality risk in participants with PAH;
to evaluate the effect of the long-term use of TPIP on the clinical status of participants with PAH;
to evaluate the effect of the long-term use of TPIP on the clinical worsening rate in participants with PAH;
to further evaluate the PK profile of the long-term use of TPIP in participants with PAH;
to explore population PK and population PK/PD relationships of the long-term use of TPIP.
to explore safety and tolerability of 720-1280 μg treprostinil palmitil.

Primary Endpoint: the frequency and severity of treatment-emergent adverse events (TEAEs) during the study.

### Secondary Endpoints:

change from pre-OLE baseline* to Month 6, Month 12, Month 18, and Month 24 in 6MWD (absolute and relative);
change from pre-OLE baseline* to Month 6, Month 12, Month 18, and Month 24 in the concentration of NT-proBNP in blood;
change from pre-OLE baseline* to Month 6, Month 12, Month 18, and Month 24 in REVEAL Lite 2.0 score;
change from pre-OLE baseline* to Month 6, Month 12, Month 18, and Month 24 in NYHA/WHO functional capacity class;
annualized rate of clinical worsening events—clinical worsening events are defined as one of the following:
   all-cause death, or onset of TEAE with a fatal outcome occurring ≤14 days after study drug discontinuation;
   hospitalization for right heart failure (for >48 hours);
   heart-lung or lung transplant, or atrial septostomy;
   addition (or increase in dose) of specified PAH-specific medications;
combined occurrence of events including a 20% decrease in 6MWD, worsening;
WHO/NYHA functional capacity class, and appearance of or worsening of signs/symptoms of right heart failure.
annualized clinical worsening event rate defined as the total number of clinical worsening events that occurred during the treatment period divided by the total number of participant-years during the treatment period;
plasma concentration levels of TP and TRIE;
PK parameters of TP and TRE are determined via population PK analysis;
PK-efficacy (e.g., 6MWD, NT-proBNP, hemodynamic changes) and PK-safety (e.g., cough, hypotension) relationships are explored via population PK analysis.

### Study Drug

Treprostinil palmitil (TP) is an inactive prodrug of treprostinil (TRE), which is widely used in the treatment of pulmonary hypertension (PH). Treprostinil palmitil inhalation powder (TPIP) is being developed for the treatment of PAH (WHO Group 1 PH). Treprostinil palmitil inhalation powder is expected to provide extended release of TRE in the lung with the goal of enabling less frequent, more convenient, and consistent dosing, whilst limiting side effects.

Capsules containing 1 of 3 dosage strengths of TPIP (80 μg, 160 μg, or 320 μg) or placebo are the study drugs. Study drug is taken around the same time every day. Administration of study drug is via inhalational dry powder packaged in single actuation capsules. One to four capsules are administered for each dose. Administration of two or more capsules is done sequentially. Each TPIP capsule contains 8 mg, 16 mg, or 32 mg of dry powder containing 80 μg, 160 μg, or 320 μg of the TP prodrug, respectively. Matching placebo capsules for TPIP are also provided for the 3-week blinded titration period.

TABLE 9
Study Drug

| | |
|---|---|
| Study Drug | TPIP |
| Dose Formulation | Dry Powder Capsule |
| Unit Dose Strengths | 80 μg, 160 μg, 240 μg, 320 μg, 400 μg, |
| | 480 μg, 640 μg, 800 μg, 960 μg, 1120 |
| | μg, or 1280 μg QD |
| Dosage Levels | 1-4 capsules |
| Packaging and Labeling | TPIP-filled HPMC capsules are delivered |
| | in HDPE bottles |

### Study Design

The present study is a Phase 2/3 OLE study of TPIP in participants with PAH from lead-in studies INS1009-201 (open-label), INS1009-202 (randomized, double-blind, placebo-controlled) and other lead-in studies of TPIP in participants with PAH, to assess the safety, tolerability, and effectiveness of the long-term use of TPIP in participants with PAH. This study also includes PK endpoints to further evaluate the PK profile of the long-term use of TPIP and exploratory endpoints for population PK and population PK/PD analyses.

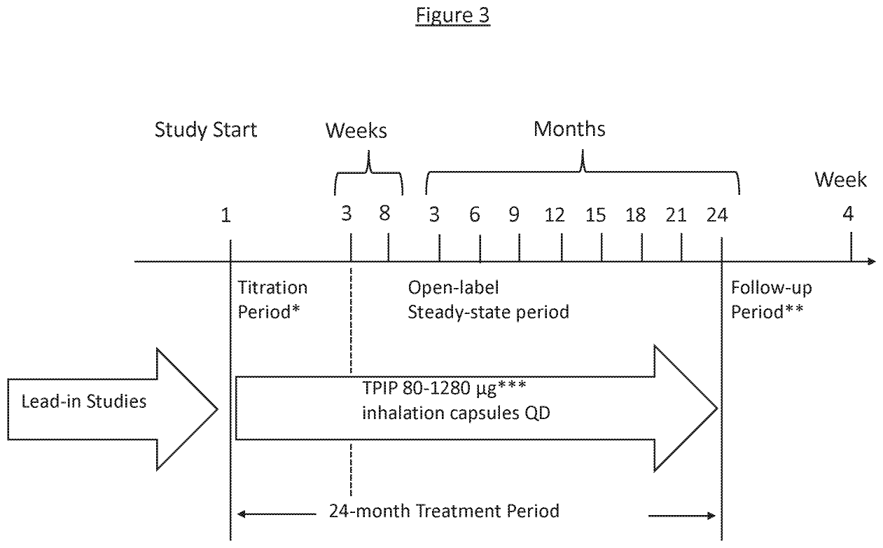

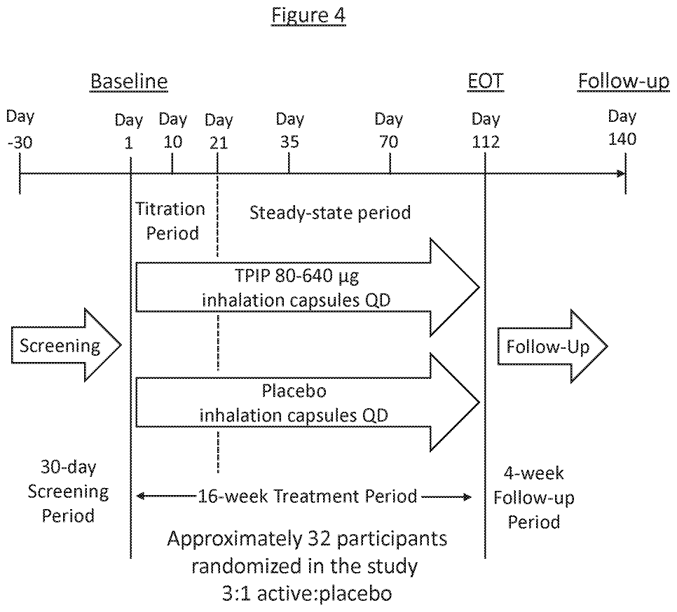

As individual tolerability varies with other prostacyclin receptor agonists, a titration period is employed in this study for all participants. Following a 3-week titration period, each participant receives open-label TPIP QD for a period of up to 24 months (inclusive of a 3-week titration period), which is followed by a 4-week follow-up. FIG. 3 provides an overall schema for the study.

Participants who have previously completed an end of treatment visit and are not transitioning immediately from a lead-in TPIP study (i.e., those no longer on study medication, having completed their lead-in end of treatment visit within one year prior to enrolment in the OLE study) undergo open-label TPIP dose titration from a starting dose of 80 μg TPIP QD to the lesser of the maximum tolerated dose or 640 μg TPIP QD within the 3-week titration scheme. In the event that a participant transitions immediately from an open-label lead-in study, where a 3-week TPIP titration has been performed, they commence the OLE study (Day 1) at their achieved TPIP dose from the lead-in study and not undergo dose increments during the 3-week titration period. For events of suspected disease progression or suspected clinical worsening, participants may have their TPIP dose increased higher than 640 μg QD, up to a maximum dose of 1280 μg QD after the initial titration period.

The study treatments are administered by oral inhalation using a Plastiape capsule-based dry powder inhaler (RS01 Mod 7). Daily doses of TPIP ranging from 80 μg to a maximum of 640 μg are achieved during the titration period with a combination of dry powder capsules containing 80 μg, 160 μg, or 320 μg of TPIP.

### Drug Administration—Titration Period

The target TPIP dose is achieved with a combination of dry powder capsules containing 80 μg, 160 μg, or 320 μg of treprostinil palmitil (IT).
During the study titration period, each participant's TPIP dose is up-titrated to the maximum tolerated TPIP dose for that individual within the titration scheme (range from 80 μg QD to a maximum of 640 μg QD). Participants start study drug with 1 capsule (80 μg TPIP) QD. If this dose is well tolerated, the dose should be up-titrated until reaching the participant's maximum tolerated dose (FIG. 2). During the titration period, participants stay on TPIP for the minimum number of cumulative days required for each dose (i.e., 2 days at 80 μg, 160 μg, or 240 μg; 3 days at 320 μg; or 4 days at 400 μg or 480 μg) prior to starting the next higher dose.
If a dose is not tolerated, TPIP may be decreased to the previous dose level. Up-titration may resume until the participant's maximum tolerated TPIP dose is achieved at Day 21.

### Drug Administration—Steady State Period

In general, the maximum dose administered during the titration period is continued in the steady state period.
Based on tolerability, the TPIP dose may be increased by 1 dose level (as described in FIG. 2) at in-clinic visits. Based on tolerability, the TPIP dose may be increased further at subsequent visits (scheduled or unscheduled) up to the maximum permitted TPIP dose of 1280 μg QD.
In the case of suspected disease progression or suspected clinical worsening, participants may have their TPIP dose increased higher than 640 μg QD, up to a maximum dose of 1280 μg QD. The increase is administered in 160 μg increments.
   Examples of reasons for increasing TPIP dose higher than 640 μg QD:
      Suspected disease progression
      Suspected clinical worsening
      Suspected reduced efficacy
Once the individual maximum tolerated dose of TPIP (doses ranging from 80 μg QD to 1280 μg QD) is identified, participants are treated with their individual maximum tolerated dose of TPIP throughout the steady state period.

### Study Assessments and Procedures

Exercise Capacity (6-Minute Walk Test)—One 6-minute walk test (6MWT) is performed at baseline and at Month 6, Month 12, Month 18, and Month 24/early discontinuation prior to administration of TPIP, collecting 6MWD values only. The 6MWD value from the Day 1 6MWT is used as the baseline value for the OLE study. The 6MWD value from the lead-in study may be recorded as the baseline value for the OLE study if performed within 7 days prior to Day 1. Pre-OLE baseline (defined as the baseline of the lead-in TPIP study) 6MWD scores is collected, if available, for each participant from their previous lead-in TPIP study. The standardized protocol based on the American Thoracic Society (ATS) guidelines (American Thoracic Society, 2002) is used.
The REVEAL Lite 2.0 risk assessment is calculated at baseline and at Month 6, Month 12, Month 18, and Month 24/early discontinuation. REVEAL Lite 2.0 provides a simplified method for assessment of low, intermediate, and high risk of 1-year mortality in PAH patients.
NT-proBNP—Brain natriuretic peptide and NT-proBNP are widely used diagnostic biomarkers for heart failure and cardiac dysfunction in clinical medicine. Blood samples for NT-proBNP monitoring are taken at baseline and at Month 6, Month 12, Month 18, and Month 24/early discontinuation prior to administration of TPIP. The NT-proBNP value from Day 1 are used as the baseline value for the OLE study. The NT-proBNP value from the lead-in study may be recorded as the baseline value for the OLE study if performed within 7 days prior to Day 1. Pre-OLE baseline (defined as the baseline of the lead-in TPIP study) NT-proBNP values are collected for each participant from their previous lead-in TPIP study.
Functional status—change in NYHA/WHO functional capacity class (World Health Organization, 2020) is assessed at baseline and at Month 6, Month 12, Month 18, and Month 24/early discontinuation prior to administration of TPIP. The NYHA/WHO functional capacity class from Day 1 is used as the baseline for the OLE study. Assessment of functional capacity class from the lead-in study may be recorded as the baseline value for the OLE study if performed within 7 days prior to Day 1. Pre-OLE baseline (defined as the baseline of the lead-in TPIP study) NYHA/WHO functional capacity class is collected for each participant from their previous lead-in TPIP study.
Clinical worsening—clinical worsening is assessed for each participant from baseline through Month 24/early discontinuation. The Investigator evaluates the participant for potential clinical worsening throughout the study. If clinical worsening is suspected, additional clinic evaluations may be required to make a final determination as to whether clinical worsening has been met.
Pre-OLE baseline (defined as the baseline of the lead-in TPIP study) details of events of clinical worsening, including prescribed PAH medications, are collected for each participant from their previous lead-in TPIP study.
   Clinical worsening is defined as one of the following:
      All-cause death, or onset of TEAE with a fatal outcome occurring ≤14 days after study drug discontinuation.
      Hospitalization for right heart failure (for >48 hours), heart-lung or lung transplant, or atrial septostomy.
      The participant requires the addition (or increase in dose if applicable) of any of the following PAH-specific medications:
         prostacyclin/prostacyclin analogue (IV, SC, oral, or inhaled),
         PDE5 inhibitors,
         soluble guanylate cyclase stimulator,
         endothelin receptor antagonist.
      The combined occurrence of the events listed below:
         a decrease in 6-MWD by at least 20% from baseline, confirmed on two 6MWTs on 2 separate days,
         increase (worsening) in NYHA/WHO functional capacity class (World Health Organization, 2020) from baseline,
         appearance of or worsening of signs/symptoms of right heart failure that did not respond to optimized oral diuretic therapy.

Safety Assessments include physical examination, vital signs, electrocardiogram, clinical safety laboratory tests (e.g., serology tests, pregnancy testing). Drug-induced liver injury (DILI) is assessed. In patients with normal liver test results at baseline, any of the following are indicative of DILI once other causes of liver injury are excluded:
Alanine aminotransferase (ALT) equal or greater than 5× upper limit of normal (ULN);
ALT equal or greater than 3×LLN, and total bilirubin >2×ULN, and no or minimal elevations in alkaline phosphatase (ALP);
ALP equal or greater than 2×ULN when the source of increased ALP levels in the liver.

Dosing with study medication should be temporarily discontinued or stopped in participants with unexplained increases in transaminases (ALT or aspartate aminotransferase [AST]) or total bilirubin as follows:
ALT or AST>5×ULN
ALT or AST>5×ULN for more than 2 weeks
ALT or AST>3×ULN and (total bilirubin >2×ULN or international normalized ratio >1.5)
ALT or AST>3×ULN with the appearance of fatigue, nausea, vomiting, right upper quadrant pain or tenderness, fever, rash, and/or eosinophilia (>5%).

Pharmacokinetic Assessments—Blood samples are for measurement of plasma concentrations of TP and TRE are done on Day 1 (pre-dose and post-dose sampling) and Month 6 (pre-dose and post-dose), Month 12 (pre-dose), Month 18 (post-dose) and Month 24 (pre-dose).

### AE Analysis

The number and percentage of AEs are presented. Treatment-emergent AEs, SAEs, AEs leading to treatment and/or study withdrawal, and AEs leading to death are listed for each participant along with outcome, severity, and relatedness to study drug. All AEs reported during the study are listed. All post-baseline clinical laboratory values, vital signs, or ECG parameters that meet the criteria for AEs are reported as such.
Definition of AE—any untoward medical occurrence in a clinical study participant, temporally associated with the use of study treatment, whether or not considered related to the study treatment. An AE can therefore be any unfavorable and unintended sign (including an abnormal laboratory finding), symptom, or disease (new or exacerbated) temporally associated with the use of study treatment.
A TEAL is defined as any AE that occurs after the first dose of study IMP and within 28 days after the last dose of study IMP.
Events Meeting the AE Definition:
   Any abnormal laboratory test results (hematology, clinical chemistry, or urinalysis) or other safety assessments (e.g., ECG, radiological scans, vital signs measurements), that worsen from baseline, are considered clinically significant in the medical and scientific judgment of the Investigator (i.e., not related to progression of underlying disease), that are associated with signs and/or symptoms, require therapeutic intervention, or lead to discontinuation of the administration of study IMP must be reported as an AE.
   Exacerbation of a chronic or intermittent pre-existing condition including either an increase in frequency and/or intensity of the condition.
   New conditions detected or diagnosed after study IMP administration even though it may have been present before the start of the study.
   Signs, symptoms, or the clinical sequelae of a suspected IMP-IMP interaction.
   Signs, symptoms, or the clinical sequelae of a suspected overdose of either study IMP or a concomitant medication. Overdose per se is not reported as an AE/SAE unless it is an intentional overdose taken with possible suicidal/self-harming intent. Such overdoses must be reported regardless of sequelae.
   The signs, symptoms, and/or clinical sequelae resulting from lack of efficacy are reported as AE or SAE if they fulfill the definition of an AE or SAE. “Lack of efficacy” or “failure of expected pharmacological action” also constitutes an AE or SAE (e.g., an event that results in death, is life-threatening, requires inpatient hospitalization or prolongation of existing hospitalization, results in persistent or significant disability/incapacity, is a congenital anomaly/birth defect, is a suspected transmission of any infectious agent via an authorized medicinal product, etc.).

### Example 3: Phase 2, Randomized, Double-Blind, Multicenter, Placebo-Controlled Study to Evaluate the Safety and Tolerability of Treprostinil Palmitil Inhalation Powder (TPIP) in Participants with Pulmonary Hypertension Associated with Interstitial Lung Disease

### Background

Interstitial lung disease encompasses a heterogeneous group of parenchymal lung diseases that are characterized by significant scarring or fibrosis of the bronchioles and alveolar sacs within the lungs. Increased fibrotic tissue in ILD prevents oxygenation and free gas exchange between the pulmonary capillaries and alveolar sacs. The symptomatology of ILD is non-specific, and covers a wide range of symptoms, whose severity can vary substantially among patients. ILD is characterized by reductions in DLCO and fibrosis on imaging studies, such as high-resolution chest CT.

PH, a heterogeneous set of diseases defined by elevation of pulmonary arterial pressure (PAP; mean PAP>20 mm Hg) and pulmonary vascular resistance (PVR; at least 3 Wood Units), is driven by pathological remodeling of the pulmonary arteriolar vasculature. PH is observed in an estimated 30-50% of patients with ILDs and notably worsens the prognosis with its presence. The presumed pathophysiology of PH-ILD includes the loss of lung tissue and thus a reduction in the total vascular bed capacity, or hypoxic vasoconstriction.

Primary Objective: To evaluate the safety and tolerability of TPIP compared with placebo in participants with PH-ILD.

### Secondary and Exploratory Objectives:

to assess the PK of TP and TRE in plasma;
to assess the effect of TPIP compared with placebo on exercise capacity;
to assess the effect of TPIP compared to placebo on clinical worsening;
to assess the effect of TPIP compared with placebo on quality of life (QOL);
to assess the effect of TPIP compared with placebo on the concentration of NT-proBNP in blood;
to evaluate the effect of TPIP compared with placebo on pulmonary vasculature blood flow and lung parenchyma;
to assess the correlation between PIFR and lung function parameters at screening.

### Primary Endpoints:

frequency and severity of TEAEs and SAEs during the study;
change in oxygenation, as measured by pulse oximetry, pre-, during, and
post-6MWT at Baseline, Week 5, Week 10, and Week 16.

### Secondary Endpoints:

plasma PK parameters of TP and TRE, such as Cₘₐₓ, Tₘₐₓ, AUC₂₄, CL/F, t_{1/2}, and Vd/F;
change from Baseline in trough 6MWT distance at Week 5, Week 10, and Week 16;
percent change from Baseline in trough 6MWT distance at Week 5, Week 10, and Week 16;
absolute and relative change from Baseline in ppFVC, FVC, ppFEV1, FEV1, FEV25-75%, at Week 2, Week 3, Week 5, Week 10, and Week 16;
change from Baseline in TLC, and DLCO at Week 5, Week 10, and Week 16;
proportion of participants experiencing clinical worsening from Baseline through Week 16;
   clinical worsening events are defined as one of the following:
      hospitalization due to a cardiopulmonary indication;
      lung transplantation;
      death from any cause;
      decrease in 6MWD≥15% from Baseline, directly related to disease under study, at 2 consecutive visits at least 24 hours apart.
change from Baseline in CAMPHOR score at Week 5, Week 10, and Week 16;
change from Baseline in the concentration of NT-proBNP at Week 5 and Week 16;
change from Baseline in total blood volume, blood volume in small vessels, lobar volume, airway volume, and airway resistance at Week 16;
parameters at screening, including PIFR, FEV, FVC, TLC, IC, and FRC.

### Study Drug

Treprostinil palmitil (TP) is a hexadecyl ester prodrug of treprostinil (TRE). In the lung, TP is hydrolyzed by esterases to TRE and hexadecanol. Nonclinical studies of TP (in vitro and in vivo) demonstrated the following:
TP was converted from the prodrug to the active drug, treprostinil,
TP administration exhibited sustained delivery of treprostinil to the lungs and systemic circulation,
TP exhibited a prolonged PK and PD profile, and was efficacious in a rodent model of PAH to inhibit its associated adverse pathology and pathophysiology,
TP did not demonstrate concerning preclinical safety or toxicological findings.

Treprostinil palmitil inhalation powder (TPIP) is being developed for the treatment of PH-ILD. TPIP is a formulation of TP designed to provide sustained release of TRE in the lung over a prolonged period, thus providing prolonged vasodilation in the lung vasculature. The prolonged lung exposure and pharmacological action of TRE achieved with sustained release of TRE from the prodrug TP aims to provide more convenient and consistent dosing and reductions in dose-limiting side effects driven by fluctuations of plasma levels of TRE and associated with the currently approved forms of TRE, Tyvaso® and Tyvaso DPI®.

TABLE 10
Study Drug

| | |
|---|---|
| Study Drug | TPIP |
| Dose Formulation | Dry Powder Capsule |
| Unit Dose Strengths | 80 μg, 160 μg, or 320 μg TP per capsμle |
| Dose Regiment | 8 0 μg, 160 μg, 240 μg, 320 μg, 400 μg, |
| | 480 μg or 640 μg QD |
| Dosage Level | 1 or 2 single capsules |
| Route of Admin. | Inhalation |
| Packaging and Labeling | TPIP-filled hydroxypropyl |
| | methylcellulose (HPMC) capsules are |
| | delivered in high density polyethylene |
| | (HDPE) bottles |

### Study Design

The present study is a Phase 2, randomized, double-blind, multicenter, placebo-controlled study to evaluate the safety and tolerability TPIP in participants diagnosed with PH-ILD, with secondary endpoint for PK and exploratory endpoints for efficacy and PD. Approximately 32 participants are randomly assigned 3:1 to TPIP or matching placebo.

For individual participants, the study consists of a Screening Period of up to 30 days, a Treatment Period of 16 weeks (includes a 3-week Titration Period) and a 4-week Follow-up Period after the Week 16 visit. At the Investigator's discretion, participants may undergo a study drug taper for up to 2 weeks after the Week 16 visit to help facilitate a safe withdrawal from study drug.

### Study Drug Administration

In the present study, capsules containing 1 of 3 dosage strengths of TPIP (80 μg, 160 μg, or 320 μg treprostinil palmitil) or placebo are administered. Study drug is instructed to be taken around the same time every day. Administration of study drug is via inhalational dry powder packaged in single actuation capsules. Administration of multiple capsules is done sequentially. Each TPIP capsule contains 8 mg, 16 mg or 32 mg of dry powder containing 80 μg, 160 μg, or 320 μg of the TP prodrug, respectively. A matching placebo capsule for TPIP is also provided.

The treprostinil palmitil (TP) dose levels are achieved with a combination of dry powder capsules containing 80 μg, 160 μg, and 320 μg of TP. There is a 21-day dose-titration period beginning at Baseline during which the TP dose is titrated up to the participant's maximum tolerated dose.

During the Titration Period (Days 1 to 21), each participant's dose is uptitrated to the highest tolerated dose for that individual. Participants start study drug with 1 capsule (80 μg TP or placebo) QD. If this dose is well tolerated, the dose is uptitrated until reaching the participant's highest tolerated dose as described by the dosing schedule in FIG. 2. During the Titration Period, participants must stay on Study Drug for the minimum number of cumulative days required for each dose (i.e., 2 days at 80 μg, 160 μg, or 240 μg, 3 days at 320 μg or 4 days at 400 μg or 480 μg) prior to starting the next higher dose. Study drug titration may occur slower, but not faster, than described in FIG. 2. If a dose is not tolerated, study drug is decreased to the previous dose level. At the Investigator's discretion, uptitration may resume (as described in FIG. 2) until the participant's highest tolerated dose is achieved at Day 21.

Based on tolerability, the TP dose may be increased by 1 dose level at in-clinic visits. The increase in dose is administered under clinical observation at the study site to ensure tolerability and correct dosing and self-administration of TPIP. Based on tolerability, the TP dose may be increased further at subsequent visits (scheduled or unscheduled) up to the maximum permitted TP dose of 640 μg QD.

In the case of suspected disease progression or suspected clinical worsening, participants may have their TP dose increased higher than 640 μg QD, up to a maximum dose of 1280 μg QD. The increase is administered in 160 μg increments.
Examples of reasons for increasing TPIP dose higher than 640 μg QD may include:
   Suspected disease progression
   Suspected clinical worsening
   Suspected reduced efficacy

### Study Duration

Including the Screening Period of up to 30 days, the Treatment Period of 16 weeks, and a 4-week Follow-up Period, the total duration of study participation for each participant is approximately 24 weeks. Screening assessment includes the following:
Informed consent
Safety laboratory samples (primarily to exclude pregnancy)
Assess Inclusion/Exclusion criteria/Demographics
Vital signs/ECG/Physical Examination/Medical History
PIFR
Spirometry/Plethysmography
6MWT (Includes Borg Scale)
Chest CT scan
RHC (if not already obtained within 90 days prior to Screening Visit)

### Treatment Duration

For an individual participant, the treatment duration is 16 weeks, including a 3-week uptitration period, followed by an optional down-titration period of 2 weeks after the Week 16 visit. The optional 2-week down-titration period is at Investigator discretion to ensure safe withdrawal from the study drug.

### Study Assessments and Procedures

Right Heart Catheterization (RHC)—the RHC should be performed during the Screening Period or within 90 days prior to the Screening Visit. Screening RHC values are used to confirm the presence of PH. Pressure, saturation, and cardiac output readings (PVR, CO; PAP; PCWP; RAP; RVP; MVO2, and SVR) are performed at each time point. Following completion of protocol-required assessments, standard of care protocols for participant follow-up for an RHC procedure apply.

Exercise Capacity (6-Minute Walk Test)—The 6MWT is performed at Screening and at Week 5, Week 10, and Week 16/Early Discontinuation prior to administration of study drug. The 6MWT values from the Screening 6MWT (6MWD, oxygenation/SpO2) are used as Baseline values. Pretest assessments are performed for heart rate, blood pressure, SpO2, and dyspnea, and overall fatigue using the Borg scale. Pretest assessment parameters are repeated after exertion. The maximum distance achieved and post exertion heart rate, blood pressure, SpO2, and dyspnea are compared to pretest values.

Quality of Life: CAMPHOR Questionnaire—the CAMPHOR should be completed as the first assessment during these visits (after informed consent is obtained) before the participant completes any of the other scheduled visit assessments. The CAMPHOR is a disease-specific assessment tool used for the evaluation and follow-up of patients with PH. The CAMPHOR Questionnaire consists of 3 sections evaluating a total of 65 items (25 relating to symptoms, 15 relating to activities, and 25 relating to QOL). The CAMPHOR scoring is negatively weighted therefore, a higher score indicates worse QOL and greater functional limitation. Symptom and QOL items are both scored out of 25 and activity items have 3 possible responses (score 0-2), giving a score out of 30.

Pulmonary Function—change in pulmonary function is assessed with spirometry (FEV1, FVC, FEV25-75%), plethysmography (TLC), and DLCO testing. Spirometry is performed per the ATS/ERS criteria (Miller et al., 2019).
The following lung function variables are collected:
   FVC and % predicted
   FEV and % predicted
   FEF25-75%
   TLC and % predicted
   RV and % predicted
   IC and % predicted
   DLCO and % predicted
   DLCO/VA and % predicted
   FRC
The predicted spirometry values are calculated according to the GLI 2012 standard and the predicted lung volumes are calculated according to Quanjer 1993 standards.

Clinical worsening—clinical worsening is assessed for each participant. The Investigator evaluates the participant for potential clinical worsening throughout the study.
Clinical worsening is defined as one of the following:
   Hospitalization due to a cardiopulmonary indication
   Lung transplantation
   Death from any cause
   Decrease in 6MWD≥15% from Baseline, directly related to disease under study, at 2 consecutive visits at least 24 hours apart
   Need for additional PH therapy

CT Scan for Assessment of Pulmonary Vasculature, Blood Flow, and Lung Parenchyma—functional respiratory imaging (FRI) using computerized tomography (CT) for assessing vascular volume, pulmonary mechanics, and airway volumes are completed at Screening and Week 16/Early Discontinuation if applicable. FRI Screening values are used as Baseline values for relevant endpoints.

### Safety Assessments

Complete Physical Examination—the complete physical examination, which includes evaluation of the head (external), eyes, ears, nose and throat, lungs, cardiovascular system, abdomen, musculoskeletal system, skin, lymph nodes, central nervous system is performed at Screening and prior to study drug administration at Baseline and the End of Treatment Visits. The physical examination also includes measurement of height, body weight, and calculation of BMI
Screening: The complete physical examination determines the individual participant's eligibility to safely participate in the study and results are documented accordingly.
Baseline: The physical examination also focuses on the participant's eligibility to participate in the study as well as their fitness for the study procedures and study drug administration.

Targeted Physical Exam—the targeted physical examination includes cardiovascular (cardiac auscultation, assessment of peripheral pulses, and edema) and pulmonary assessments (pulmonary auscultation) performed prior to study drug administration.

Vital Signs—vital signs including a 5-minute sitting BP (systolic, diastolic blood, and mean arterial pressures), heart rate (bpm), body temperature (°C), respiratory rate (breaths per minute), and SpO2 (pulse oximetry) are assessed before blood collection for laboratory tests prior to study drug administration. Blood pressure and pulse measurements are assessed after the participant has been seated for 5 minutes

Oxygenation—oxygenation (SpO₂) and heart rate are assessed with continuous pulse oximetry prior to, during, and after each 6MWT (Section 8.1.2) as specified in the 6MWT manual. The SpO₂ at rest and the lowest SpO₂ value recorded during the 6MWT are recorded as data points in the CRF.

Electrocardiogram—a single 12-lead ECG is conducted. ECG assessments include comments on whether the tracings are normal or abnormal, rhythm, presence of arrhythmia or conduction defects, morphology, any evidence of myocardial infarction, or ST-segment, T-wave, and U wave abnormalities. In addition, measurements of the following intervals are measured and reported: RR interval, PR interval, QRS width, QT interval, and QT interval corrected for heart rate using Fridericia's formula. ECGs are read/assessed locally by the Investigator or qualified designee.

Pharmacokinetic Assessments—Blood samples of approximately 10 mL each are collected for measurement of plasma concentrations of TP and TRE. At Baseline (Day 1) and at Week 10, PK samples are collected at 0 (pre-dose), 0.5 hour (±5 min), 1 hour (±10 min), 2 hours (±10 min), 4 hours (±30 min), and 8 hours (±2 hours) post-dose. Approximately 24 hours (±4 hours) after the Week 10 Visit (V6+1), the participant returns to the clinic prior to administering that day's dose of study drug, for a pre-dose PK sample. The participant MUST NOT administer that day's dose of study drug prior to the pre-dose PK blood sample. At Week 2 and Week 5, PK samples are collected at 0 (pre-dose) and at 2 hours (±10 min) post-dose. At Week 16, the PK sample is collected at 0 (pre-dose) only. A PK sample should be collected at the Early Discontinuation Visit when feasible.

Individual PK parameters of TRE and TP are determined using noncompartmental analysis for the following parameters: Cₘₐₓ, Tₘₐₓ, AUC_{inf}, AUC₂₄, AUCₗₐₛₜ, t_{1/2}, CL/F, and Vd/F based on the concentration data from Baseline (Day 1), Week 2, Week 5, and Week 10, as appropriate. In addition, accumulation of Rac(cₘₐₓ) and Rac(_{AUC}) are evaluated based on the ratios between steady-state parameters (Week 10) and single-dose parameters at Baseline (Day 1), if calculable.

NT-proBNP Biomarker—Blood samples (4 mL per time point) for measuring NT-proBNP are collected at Baseline (Day 1) and at Week 5, Week 10, and Week 16 (or at the Early Discontinuation Visit, if applicable) prior to study drug administration. The plasma generated from the blood samples is stored frozen (−70 °C or lower is preferred) until shipment and analysis. The estimated total blood loss for NT-proBNP sampling is approximately 16 mL.

### Primary Endpoint Analysis

The primary endpoint is frequency and severity of TEAEs and SAEs during the study. Other safety endpoints include changes in oxygenation (SpO2) as measured by continuous pulse oximetry prior to, during, and after the 6MWT at Baseline, Week 5, Week 10, and Week 16.

The number and percent of AEs are presented by treatment and dose group. TEAEs, SAEs, AEs leading to treatment and/or study withdrawal, AEs leading to death are listed for each participant along with outcome, severity, and relatedness to study drug. TEAEs and SAEs are summarized by SOC and PT and individual preferred term within each SOC, by treatment group in the descending order of incidence. All AEs reported during the study are listed.

All post-Baseline clinical laboratory values, vital signs, or ECG parameters that meet the criteria for AIs are reported as such.

All clinical laboratory values (blood chemistry and hematology) are listed for each participant. Laboratory values outside normal ranges are flagged. If applicable, summary statistics (mean, range) for actual values and change from Baseline by treatment are provided for each time point. Baseline is defined by laboratory values from blood samples collected prior to the first dose at the Baseline visit. Summary statistics of clinical laboratory values are tabulated by treatment and dose.

Vital signs (systolic, diastolic, and mean BP, heart rate, respiratory rate, SpO2, temperature) and body weight, including changes from Baseline, are summarized for the double-blind Treatment Period over time, by treatment group, for each parameter using descriptive statistics for continuous data. In each evaluation, only participants who had both baseline and postbaseline assessments are included. All the above analyses are performed regardless of the location (left arm, right arm) or the position (supine or sitting) of the BP measurement.

The results of the safety ECGs reported for the double-blind Treatment Period are listed for each participant. Postbaseline findings of ECG results that meet the criteria for AEs are listed in the relevant AE listings. If applicable, summary statistics of ECG results are provided.

The mixed model for repeated measures (MMRM) is applied to Change from Baseline in resting SpO2 during 6MWT at Weeks 5, 10, and 16 and Change from Baseline in peak exercise SpO₂ during 6MWT at Weeks 5, 10, and 16 endpoints.

### Secondary Endpoint Analysis

Individual PK concentrations at each sampling time and PK parameters of TRE and TP are provided in listings. Plasma concentrations of TRE and TP are summarized for mean, SD and CV using descriptive statistics. For PK parameter calculations, pre-dose BLQ data on Day 1 are treated as zero. All post-dose BLQ data on Day 1 and Week 10 are treated as missing for PK parameter calculations. Cₘₐₓ, Tₘₐₓ, AUC₂₄, AUC_{inf}, AUCₗₐₛₜ, t_{1/2}, CL/F and Vd/F, and all other parameters for both TRE and TP are provided, if calculable, and summarized based on dose levels for mean, SD, CV, and geometric mean. Tₘₐₓ are reported as median, minimum, and maximum. Rac(cₘₐₓ) and Rac(_{AUC}) are evaluated, if calculable, at Week 10.

Dose linearity in Cₘₐₓ and AUC on Day 1 and Week 10 is evaluated if dose titration results in 3 or more steady-state dose levels in the study.

Analysis for 6MWD at Week 5, 10, and 16—the 6MWD value from the Screening 6MWT is used as the Baseline value. Change and percent change from Baseline in 6MWD are summarized by time point and treatment group using descriptive statistics. Percent change from Baseline to each time point is summarized similarly. Data are analyzed using the MMRM with factors for treatment group, time point, treatment by time interaction, and covariates for Baseline 6MWD distance for the ITT population. An unstructured covariance matrix is used to account for the correlation between repeated measurements from the same participant.

Change from Baseline in ppFVC, FVC, ppFEV1, FEV1, FEV25-75%, TLC, and DLCO at Week 5, Week 10, and Week 16.

Proportion of Participants Experiencing Clinical Worsening from Baseline Through Week 16.

Change from Baseline in CAMPHOR Score at Week 5, Week 10, and Week 16.

Change from Baseline in total blood volume, blood volume in small vessels, lobar volume, airway volume, and airway resistance at Week 16.

Change from Baseline in Plasma Concentrations of NT-proBNP at Week 5, Week 10, and Week 16—NT-proBNP is summarized by time point and treatment group using descriptive statistics as well as geometric means and CV. Change from Baseline in NT-proBNP at Week 5, Week 10, and Week 16 is summarized similarly. Change from Baseline in NT-proBNP is log-transformed and analyzed using MMRM.

### AE Analysis

The number and percentage of AEs are presented. Treatment-emergent AEs, SAEs, AEs leading to treatment and/or study withdrawal, and AEs leading to death are listed for each participant along with outcome, severity, and relatedness to study drug. All AEs reported during the study are listed. All post-baseline clinical laboratory values, vital signs, or ECG parameters that meet the criteria for AEs are reported as such.
Definition of AE—any untoward medical occurrence in a clinical study participant, temporally associated with the use of study treatment, whether or not considered related to the study treatment. An AE can therefore be any unfavorable and unintended sign (including an abnormal laboratory finding), symptom, or disease (new or exacerbated) temporally associated with the use of study treatment.
A TEAE is defined as any AE that occurs after the first dose of study IMP and within 28 days after the last dose of study IMP.
Events Meeting the AE Definition:
   Any abnormal laboratory test results (hematology, clinical chemistry, or urinalysis) or other safety assessments (e.g., ECG, radiological scans, vital signs measurements), that worsen from baseline, are considered clinically significant in the medical and scientific judgment of the Investigator (i.e., not related to progression of underlying disease), that are associated with signs and/or symptoms, require therapeutic intervention, or lead to discontinuation of the administration of study IMP must be reported as an AE.
   Exacerbation of a chronic or intermittent pre-existing condition including either an increase in frequency and/or intensity of the condition.
   New conditions detected or diagnosed after study IMP administration even though it may have been present before the start of the study.
   Signs, symptoms, or the clinical sequelae of a suspected IMP-IMP interaction.
   Signs, symptoms, or the clinical sequelae of a suspected overdose of either study IMP or a concomitant medication. Overdose per se is not reported as an AE/SAE unless it is an intentional overdose taken with possible suicidal/self-harming intent. Such overdoses must be reported regardless of sequelae.
   The signs, symptoms, and/or clinical sequelae resulting from lack of efficacy are reported as AE or SAE if they fulfill the definition of an AE or SAE. “Lack of efficacy” or “failure of expected pharmacological action” also constitutes an AE or SAE (e.g., an event that results in death, is life-threatening, requires inpatient hospitalization or prolongation of existing hospitalization, results in persistent or significant disability/incapacity, is a congenital anomaly/birth defect, is a suspected transmission of any infectious agent via an authorized medicinal product, etc.).

Drug-induced liver injury (DILI) is assessed. In patients with normal liver test results at baseline, any of the following are indicative of DILI once other causes of liver injury are excluded:
Alanine aminotransferase (ALT) equal or greater than 5× upper limit of normal (ULN);
ALT equal or greater than 3×ULN, and total bilirubin >2×ULN, and no or minimal elevations in alkaline phosphatase (ALP);
ALP equal or greater than 2×ULN when the source of increased ALP levels in the liver.

Dosing with study medication should be temporarily discontinued or stopped in participants with unexplained increases in transaminases (ALT or aspartate aminotransferase [AST]) or total bilirubin as follows:
ALT or AST>8×ULN
ALT or AST>5×ULN for more than 2 weeks
ALT or AST>3×ULN and (total bilirubin >2×ULN or international normalized ratio >1.5)
ALT or AST>3×ULN with the appearance of fatigue, nausea, vomiting, right upper quadrant pain or tenderness, fever, rash, and/or eosinophilia (>5%).

### Clinical Analysis

All clinical laboratory values (clinical chemistry and hematology) are listed for each participant. Laboratory values outside normal ranges are flagged. If applicable, summary statistics (mean, range) for actual values and change from baseline are provided for each time point. Baseline is defined by the last non-missing laboratory values from blood samples collected prior to or at the date of the first dose of study drug. Summary statistics of clinical laboratory values are tabulated.

Other safety endpoints include change in oxygenation, as measured by pulse oximetry, pre-, during, and post-6MWT at Baseline, Week 5, Week 10, and Week 16. The mixed model for repeated measures (MMRM) are applied.

### Efficacy Analysis

Efficacy analyses are performed with the ITT population set. Change from Baseline in 6MWT distance is summarized by time point and treatment group using descriptive statistics. Percent change from Baseline to each time point are summarized similarly. Percent change from Baseline in 6MWT distance (Week 5, Week 10, and Week 16) is analyzed using a repeated measurements model with factors for treatment group, time point, treatment by time interaction, and covariates for Baseline 6MWT distance. An unstructured covariance matrix is used to account for the correlation between repeated measurements from the same participant. Similar analyses are used for other efficacy variables.

### Results

Patients were randomized 3:1 to TPIP or placebo dry powder inhalation once-daily, titrated from 80 μg to the maximum tolerated dose. Primary endpoints were adverse events (AEs), serious AEs (SAEs), and change in oxygenation during 6-min walk test (6MWT).

### Results

39 patients were randomized (TPIP n=29; placebo n=10); 30 (76.9%) completed treatment. Baseline characteristics were generally balanced except for gender. TPIP maximum tolerated dose (640 μg) was achieved by 79.3% of patients. AEs occurred in 27 (93.1%) vs 9 (90.0%) patients with TPIP vs placebo; most frequent were cough, diarrhea, and dyspnea; most were mild/moderate. SAEs occurred in 6 (20.7%) vs 4 (40.0%) patients with TPIP vs placebo; there were 2 deaths in each group. No SAE/death was assessed as treatment-related. AEs leading to treatment discontinuation were less frequent with TPIP (13.8%) vs placebo (30.0%). There were no meaningful changes from baseline in oxygenation at 6MWT or in supplemental oxygen use with TPIP. The TPIP PK profile supported once-daily dosing. At week 10 following TPIP 480 μg and 640 μg, the treprostinil median Tₘₐₓ, mean Cₘₐₓ, AUC_{T}, and t_{1/2} were 1.03 h and 1.46 h, 1246 and 1594 pg/mL, 4675 and 7510 pg*h/mL, and 7.28 and 7.10 h, respectively. Exploratory endpoints: clinical worsening occurred in 3 (10.3%) vs 5 (50.0%) patients with TPIP vs placebo (nominal p=0.01649). Placebo-corrected median change from baseline in the 6MWT distance with TPIP was 30 m (95% CI−49 to 171; nominal=0.34.

TPIP was safe and well tolerated at the maximum dose, achieved by the majority of patients. AEs were consistent with inhaled prostacyclin therapy.

### Functional Respiratory Imaging (FRI) Analyses

FRI parameters were calculated using high-resolution computed tomography at screening (V1) and week 16, and identical statistical analyses were performed on subjects with CT scans meeting the standard protocol described above. A description of exemplary FRI parameters evaluated are shown in Table 11 below.

TABLE 11
Exemplary FRI and Clinical Parameters

| Abbreviation | FRI Parameters |
|---|---|
| IVLOBE | Image-based lung volume (L) |
| IVLOBEPP | Percent predicted lung volume (%) |
| IVAW | Image-based airway volume (mL) |
| IVAWPP | Percent predicted airway volume (%) |
| SIVAW | Specific image-based airway volume (mL/L) |
| SIVAWPP | Percent predicted specific airway volume (%) |
| IVFIB | Image-based fibrosis score (mL) |
| SIVFIB | Specific image-based fibrosis score (%) |
| LAS | Emphysema score (%) |
| IVART | Image-based artery volume (mL) |
| IVARTPR | Image-based artery proportion (%) |
| IVVEIN | Image-based vein Volume (mL) |
| IVVEINPR | Image-based vein proportion (%) |
| SIVART | Specific image-based artery volume (%) |
| SIVVEIN | Specific image-based vein volume (%) |
| BV5 | Volume of blood vessels smaller than 5 mm2 |
| | in cross sectional area (mL) |
| BV5PR | Proportion of blood vessels smaller than 5 mm2 |
| | in cross sectional area (%) |
| BV5A | Volume of pulmonary arteries smaller than |
| | 5 mm2 in cross sectional area (mL) |
| BV5APR | Proportion of pulmonary arteries smaller than |
| | 5 mm2 in cross sectional area compared to all |
| | blood vessels (%) |
| BV5APRA | Proportion of pulmonary arteries smaller than |
| | 5 mm2 in cross sectional area compared |
| | to all arteries (%) |
| BV5V | Volume of pulmonary veins smaller than 5 mm2 |
| | in cross sectional area (mL) |
| BV5VPR | Proportion of pulmonary veins smaller than |
| | 5 mm2 in cross sectional area compared |
| | to all blood vessels (%) |
| BVSVPRV | Proportion of pulmonary veins smaller than |
| | 5 mm2 in cross sectional area compared to |
| | all veins (%) |
| BV510 | Volume of blood vessels between 5 and 10 |
| | mm2 in cross sectional area (mL) |
| BV510PR | Proportion of blood vessels between 5 and |
| | 10 mm2 in cross sectional area (%) |
| BV510A | Volume pulmonary arteries between 5 and |
| | 10 mm2 in cross sectional area (mL) |
| BV510APR | Proportion of pulmonary arteries between 5 |
| | and 10 mm2 in cross sectional area |
| | compared to all blood vessels (%) |
| BV510APRA | Proportion of pulmonary arteries between 5 |
| | and 10 mm2 in cross sectional area |
| | compared to all arteries (%) |
| BV510V | Volume pulmonary veins between 5 and 10 |
| | mm2 in cross sectional area (mL) |
| BV510VPR | Proportion of pulmonary veins between 5 and |
| | 10 mm2 in cross sectional area compared |
| | to all blood vessels (%) |
| BV510VPRV | Proportion of pulmonary veins between 5 and |
| | 10 mm2 in cross sectional area compared |
| | to all veins (%) |
| BV10 | Volume of blood vessels larger than 10 |
| | mm2 in cross sectional area (mL) |
| BV10PR | Proportion of blood vessels larger than 10 |
| | mm2 in cross sectional area (%) |
| BV10A | Volume of pulmonary arteries larger than |
| | 10 mm2 in cross sectional area (mL) |
| BV10APR | Proportion of pulmonary arteries larger |
| | than 10 mm2 in cross sectional area |
| | compared to all blood vessels (%) |
| BV10APRA | Proportion of pulmonary arteries larger |
| | than 10 mm2 in cross sectional area |
| | compared to all arteries (%) |
| BV10V | Volume of pulmonary veins larger than 10 |
| | mm2 in cross sectional area (mL) |
| BV10VPR | Proportion of pulmonary veins larger than |
| | 10 mm2 in cross sectional area compared |
| | to all blood vessels (%) |
| BV10VPRV | Proportion of pulmonary veins larger |
| | than 10 mm2 in cross sectional |
| | area compared to all veins (%) |
| BV510 | RATIO Ratio of BV5 to BV10 |
| BV510ARATIO | Ratio of BV5A to BV10A |
| BV510VRATIO | Ratio of BV5V to BV10V |
| PA | Volume of extrapulmonary arteries (mL) |
| PV | Volume of extrapulmonary veins (mL) |

14 patients were included in the analysis (TPIP n=9; placebo n=5). Volumes of the macroscopic pulmonary arteries ≤5 mm² (BV5A) and ≥10 mm² (BV10A) in cross sectional areas were analyzed and presented as a proportion of total vessel volume (PR) and proportion of total arterial volume (PRA). The specific image-based volume of fibrosis (SIVFIB) score, based on deep-learning algorithms, was defined as fibrotic volume divided by lung volume.

A summary of the results is provided in Table 12 and Table 13 below. Table 12 provides a summary of baseline, change from baseline, and percent change from baseline values for FRI parameters in total lung zone overall and stratified by treatment group. Table 13 reports results of an ANCOVA model estimation of treatment effects for each FRI parameter. For parameters involving vascular volumes, the model includes and adjustment for fibrosis volume (IVFIB) due to possible impacts from the presence of fibrosis on vascular volume assessment.

TABLE 12
Summary of Blood Volume Parameters

| | | | | | | | | | | Effect size as % |
|---|---|---|---|---|---|---|---|---|---|---|
| FRI | Lung | | | Baseline: | | CFB: | | % CFB: | | of PLCB arm |
| Parameter | Zone | Arm | n | Mean (sd) | | Mean (sd) | | Mean (sd) | | baseline mean |
| IVFIB | TOTAL | TPIP | 9 | 240.71 | (138.22) | −4.95 | (45.68) | −1.31 | (26.14) | −6.51 |
| IVFIB | TOTAL | PLCB | 5 | 296.46 | (188.69) | 10.73 | (43.11) | 8.18 | (22.79) | — |
| IVFIB | TOTAL | Overall | 14 | 260.62 | (153.23) | 0.65 | (43.78) | 2.08 | (24.54) | — |
| SIVFIB | TOTAL | TPIP | 9 | 9.31 | (6.01) | −0.70 | (2.74) | −4.58 | (30.20) | −10.49 |
| SIVFIB | TOTAL | PLCB | 5 | 8.07 | (5.46) | 0.34 | (1.80) | 13.38 | (27.96) | — |
| SIVFIB | TOTAL | Overall | 14 | 8.87 | (5.64) | −0.33 | (2.42) | 1.84 | (29.69) | — |
| BV5A | TOTAL | TPIP | 9 | 19.54 | (7.27) | 1.81 | (6.33) | 14.01 | (37.61) | 13.03 |
| BV5A | TOTAL | PLCB | 5 | 33.55 | (21.12) | −2.96 | (3.27) | −7.49 | (4.84) | — |
| BV5A | TOTAL | Overall | 14 | 24.54 | (14.78) | 0.11 | (5.79) | 6.33 | (31.49) | — |
| BV5APR | TOTAL | TPIP | 9 | 24.10 | (5.00) | 1.83 | (2.21) | 8.39 | (11.51) | 13.72 |
| BV5APR | TOTAL | PLCB | 5 | 27.58 | (8.19) | −1.89 | (3.00) | −5.46 | (10.74) | — |
| BVSAPR | TOTAL | Overall | 14 | 25.34 | (6.25) | 0.50 | (3.03) | 3.44 | (12.83) | — |
| BVSAPRA | TOTAL | TPIP | 9 | 44.42 | (8.12) | 3.40 | (5.07) | 8.04 | (11.99) | 13.59 |
| BVSAPRA | TOTAL | PLCB | 5 | 47.12 | (13.24) | −2.29 | (4.91) | −3.75 | (10.10) | — |
| BVSAPRA | TOTAL | Overall | 14 | 45.38 | (9.81) | 1.37 | (5.59) | 3.83 | (12.42) | — |
| BV10A | TOTAL | TPIP | 9 | 11.91 | (6.52) | −1.05 | (3.39) | −7.13 | (34.93) | −25.71 |
| BV10A | TOTAL | PLCB | 5 | 15.79 | (6.41) | 2.74 | (4.89) | 16.28 | (41.46) | — |
| BV10A | TOTAL | Overall | 14 | 13.30 | (6.52) | 0.30 | (4.24) | 1.23 | (37.62) | — |
| BV10APR | TOTAL | TPIP | 9 | 14.00 | (4.30) | −1.75 | (2.99) | −10.80 | (28.46) | −23.16 |
| BV10APR | TOTAL | PLCB | 5 | 14.61 | (5.20) | 1.36 | (3.24) | 16.43 | (36.98) | — |
| BV10APR | TOTAL | Overall | 14 | 14.21 | (4.45) | −0.64 | (3.34) | −1.08 | (33.21) | — |
| BV10APRA | TOTAL | TPIP | 9 | 25.74 | (6.97) | −3.28 | (4.74) | −12.03 | (23.76) | −24.76 |
| BV10APRA | TOTAL | PLCB | 5 | 24.98 | (8.48) | 2.64 | (5.91) | 18.69 | (37.87) | — |
| BV10APRA | TOTAL | Overall | 14 | 25.47 | (7.22) | −1.16 | (5.76) | −1.05 | (31.97) | — |
| BV510ARATIO | TOTAL | TPIP | 9 | 1.98 | (1.11) | 0.69 | (1.22) | 31.43 | (37.77) | 49.38 |
| BV510ARATIO | TOTAL | PLCB | 5 | 2.56 | (2.50) | −0.70 | (1.62) | −10.54 | (36.02) | — |
| BV510ARATIO | TOTAL | Overall | 14 | 2.18 | (1.66) | 0.19 | (1.48) | 16.44 | (41.38) | — |

TABLE 13
Summary of Blood Volume Parameters with Fibrosis Adjustment

| | | Treatment | | | | Effect | Effect |
|---|---|---|---|---|---|---|---|
| FRI | Lung | Effect | Standard | | | Estimate | Estimate |
| Parameter | Zone | Estimate | Error | Statistic | P-value | Lower CI | Upper CI |
| IVART | TOTAL | 3.90 | 6.81 | 0.57 | p = 0.58 | −11.27 | 19.07 |
| IVARTPR | TOTAL | −0.47 | 1.24 | −0.38 | p = 0.71 | −3.24 | 2.30 |
| IVVEIN | TOTAL | 1.62 | 6.25 | 0.26 | p = 0.80 | −12.30 | 15.55 |
| IVVEINPR | TOTAL | 0.47 | 1.24 | 0.38 | p = 0.71 | −2.30 | 3.24 |
| SIVART | TOTAL | −0.07 | 0.16 | −0.47 | p = 0.65 | −0.42 | 0.27 |
| SIVVEIN | TOTAL | −0.11 | 0.11 | −0.98 | p = 0.35 | −0.35 | 0.14 |
| BV5 | TOTAL | 23.80 | 11.02 | 2.16 | p = 0.06 | −0.74 | 48.35 |
| BV5PR | TOTAL | 3.91 | 2.76 | 1.42 | p = 0.19 | −2.25 | 10.07 |
| BV5A | TOTAL | 4.37 | 3.51 | 1.24 | p = 0.24 | −3.45 | 12.19 |
| BV5APR | TOTAL | 3.78 | 1.32 | 2.86 | p = 0.02* | 0.83 | 6.73 |
| BV5APRA | TOTAL | 6.40 | 2.89 | 2.21 | p = 0.05 | −0.05 | 12.85 |
| BV5V | TOTAL | 1.27 | 2.58 | 0.49 | p = 0.63 | −4.49 | 7.03 |
| BV5VPR | TOTAL | 0.88 | 1.46 | 0.60 | p = 0.56 | −2.38 | 4.14 |
| BV5VPRV | TOTAL | 2.87 | 2.61 | 1.10 | p = 0.30 | −2.94 | 8.68 |
| BV510 | TOTAL | 7.25 | 2.39 | 3.04 | p = 0.01* | 1.93 | 12.58 |
| BV510PR | TOTAL | 0.16 | 0.71 | 0.23 | p = 0.83 | −1.43 | 1.75 |
| BV510A | TOTAL | 1.17 | 1.56 | 0.75 | p = 0.47 | −2.30 | 4.65 |
| BV510APR | TOTAL | 0.53 | 0.93 | 0.57 | p = 0.58 | −1.55 | 2.60 |
| BV510APRA | TOTAL | 0.14 | 1.43 | 0.10 | p = 0.93 | −3.04 | 3.32 |
| BV510V | TOTAL | 0.95 | 1.40 | 0.68 | p = 0.51 | −2.17 | 4.08 |
| BV510VPR | TOTAL | −0.13 | 0.78 | −0.17 | p = 0.87 | −1.88 | 1.61 |
| BV510VPRV | TOTAL | 0.27 | 1.63 | 0.16 | p = 0.87 | −3.37 | 3.91 |
| BV10 | TOTAL | −7.42 | 6.11 | −1.21 | p = 0.25 | −21.03 | 6.19 |
| BV10PR | TOTAL | −4.48 | 2.90 | −1.54 | p = 0.15 | −10.95 | 2.00 |
| BV10A | TOTAL | −4.06 | 2.52 | −1.61 | p = 0.14 | −9.67 | 1.55 |
| BV10APR | TOTAL | −3.38 | 1.76 | −1.92 | p = 0.08 | −7.30 | 0.54 |
| BV10APRA | TOTAL | −6.19 | 3.00 | −2.06 | p = 0.07 | −12.87 | 0.50 |
| BV10V | TOTAL | −1.35 | 2.38 | −0.57 | p = 0.58 | −6.65 | 3.95 |
| BV10VPR | TOTAL | −1.19 | 1.38 | −0.86 | p = 0.41 | −4.26 | 1.89 |
| BV10VPRV | TOTAL | −2.91 | 2.83 | −1.03 | p = 0.33 | −9.22 | 3.39 |
| BV510RATIO | TOTAL | 0.51 | 0.34 | 1.51 | p = 0.16 | −0.24 | 1.27 |
| BV510ARATIO | TOTAL | 1.26 | 0.84 | 1.50 | p = 0.17 | −0.62 | 3.14 |
| BV510VRATIO | TOTAL | 0.39 | 0.33 | 1.20 | p = 0.26 | −0.34 | 1.12 |
| PA | LEFT LUNG | 0.95 | 3.67 | 0.26 | p = 0.80 | −7.22 | 9.12 |
| PA | RIGHT LUNG | 1.88 | 4.07 | 0.46 | p = 0.65 | −7.19 | 10.94 |
| PV | LEFT LUNG | 1.49 | 1.61 | 0.93 | p = 0.38 | −2.10 | 5.09 |
| PV | RIGHT LUNG | 2.46 | 2.47 | 1.00 | p = 0.34 | −3.04 | 7.96 |

There was a significant fibrosis adjusted treatment effect for TPIP vs placebo of 3.8% (P=0.02) for BV5APR and a borderline significant fibrosis adjusted treatment effect for TPIP vs placebo of 6.4% (P=0.05) for BV5APRA (Table 13). Unadjusted % change from baseline (CFB) between TPIP vs placebo in BV5APR and BV5APRA was +8.4% vs −5.5% and +8.0% vs −3.8%, respectively (Table 12). A decrease in the proportion of total blood volume and total arterial volume contained in large pulmonary arteries (BV10APR and BV10APRA, respectively) was observed with TPIP (−10.80% and −12.03%, respectively) compared to an increase observed with placebo (+16.43% and +18.69%, respectively) (Table 12). The % CFB in the BV5A:BV10A ratio was +31.4% with TPIP vs −10.54% with placebo (Table 12). There was a directional decrease from baseline in the SIVFIB score in the TPIP group vs the placebo group (effect size compared to placebo baseline, −10.49%) (Table 12).

These results suggest roughly symmetric effects in small vs large pulmonary arteries, with an increase in small arterial blood volumes and a corresponding decrease in large arterial blood volumes of roughly equal magnitude. For both BV5APRA and BV10APRA the patterns are similar in the upper and lower lung zones. The effect size estimates in venous blood volumes also follow a similar pattern. Specifically, for total BV5VPRV the effect size estimate is 2.87% with a p-value of 0.30, while for BV10VPRV the effect size is estimate is −2.91% with a p-value of 0.33 (Table 13). Although these p-values do not reach nominal significance, the estimates suggest a similar symmetry to the arterial measures in terms of increasing small vascular volumes vs decreasing large vascular volumes.

Taken together, treatment with TPIP resulted in changes in blood volume parameters at FRI consistent with peripheral vasodilation and improved pulmonary arteriole recruitment, with an observed directional decrease in fibrosis score.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

Patents, patent applications, patent application publications, journal articles and protocols referenced herein are incorporated by reference in their entireties, for all purposes.

1.-195. (canceled)

196. Aerosolized dose of a dry powder composition, wherein the dry powder composition comprises about 1280 μg of treprostinil palmitil, or a pharmaceutically acceptable salt thereof.

197. The aerosolized dose of claim 196, wherein the aerosolized dose is provided from one dry powder composition or a plurality of dry powder compositions.

198. The aerosolized dose of claim 197, wherein the aerosolized dose is provided from one dry powder composition.

199. The aerosolized dose of claim 197, wherein the aerosolized dose is provided from two dry powder compositions.

200. The aerosolized dose of claim 198, wherein each of the two dry powder compositions comprises about 640 μg treprostinil palmitil, or a pharmaceutically acceptable salt thereof.

201. The aerosolized dose of claim 197, wherein the aerosolized dose is provided from three dry powder compositions.

202. The aerosolized dose of claim 201, wherein one of the three dry powder compositions comprises about 640 μg of treprostinil palmitil, or a pharmaceutically acceptable salt thereof, and the other two of the three dry powder compositions each comprises about 320 μg of treprostinil palmitil, or a pharmaceutically acceptable salt thereof.

203. The aerosolized dose of claim 197, wherein the dry powder composition is a dry powder capsule or a dry powder blister pack.

204. The aerosolized dose of claim 196, wherein the aerosolized dose is a once daily dose.

205. A method of treating pulmonary hypertension (PH) in a patient in need thereof, comprising administering the aerosolized dose of claim 196 to the lungs of the patient.

206. The method of claim 205, wherein the PH is pulmonary arterial hypertension (PAH).

207. The method of claim 205, wherein the PH is PH associated with interstitial lung disease (ILD).

208. The method of claim 207, wherein the ILD is idiopathic pulmonary fibrosis (IPF).
